(19) United States
(12) Patent Application Publication
(10) Pub. No.: US 2024/0043805 A1
(43) Pub. Date: Feb. 8, 2024

(54) IMMORTALIZED CELL LINE FOR ENGINEERED SYNTHETIC LEATHER

(71) Applicant: VitroLabs Inc, Milpitas, CA (US)

(72) Inventors: Craig HALBERSTADT, Pleasanton, CA (US); Ying Hsuan SHIH, San Jose, CA (US)

(21) Appl. No.: 18/453,211

(22) Filed: Aug. 21, 2023

Related U.S. Application Data
(63) Continuation of application No. PCT/US2022/017355, filed on Feb. 22, 2022.
(60) Provisional application No. 63/152,256, filed on Feb. 22, 2021.

(51) Int. Cl. C12N 5/077; C12N 9/12; C07K 14/005; C14C 3/06; C14C 3/28; D06N 3/12

(52) U.S. Cl. CPC C12N 5/0656; C12N 9/1276; C12Y 207/07049; C07K 14/005; C14C 3/06; C14C 3/28; D06N 3/12; C12N 2510/04; C12N 2500/38; C12N 2501/15; C12N 2533/90; D06N 2203/02

(57) ABSTRACT

Methods, compositions, kits and systems disclosed herein can be directed to engineered cells for producing synthetic leathers, artificial epidermal layers, artificial dermal layers, layered structures, products produced therefrom and methods of producing the same.

### CROSS-REFERENCE

This application is a continuation of International Patent Application No. PCT/US2022/017355 filed Feb. 22, 2022, which claims the benefit of U.S. Provisional Application No. 63/152,256, filed Feb. 22, 2021, each of which are incorporated herein by reference in their entirety.

### SUMMARY OF THE DISCLOSURE

Disclosed herein in some embodiments is a method comprising: seeding a transfected or transduced isolated fibroblast or fibroblast-like cell onto a scaffold to form an artificial dermal layer and contacting a transfected or transduced isolated cell with a medium, wherein a transfected or transduced isolated cell can comprise an exogenous polynucleotide, wherein: an exogenous polynucleotide encodes: (i) a polypeptide which interacts with a tumor suppressor protein or fragment thereof and alters an activity of a tumor suppressor protein or fragment thereof, wherein an activity of a tumor suppressor protein or fragment thereof can be measured by an in vitro assay, or (ii) a polynucleotide that encodes a polypeptide which interacts with a tumor suppressor protein or fragment thereof; a transfected or transduced isolated cell when in contact with a medium has: (i) increased collagen production, relative to an otherwise identical cell that is not contacted with a medium, (ii) at least partially increased differentiation, relative to an otherwise identical cell that is not contacted with a medium, or (iii) any combination of (i) and (ii). In some embodiments, a method can further comprise at least partially decellularizing an artificial dermal layer to form an at least partially decellularized artificial dermal layer. In some embodiments, a method can comprise tanning an at least partially decellularized artificial dermal layer to form a synthetic leather. In some embodiments, an exogenous polynucleotide can code for an SV40 large T antigen (SV40-TAg) polypeptide, a telomerase (TERT) protein, a Bmi-1 protein, a cyclin D1 protein, a biologically active fragment of any of these, or any combination thereof. In some embodiments, an exogenous polynucleotide can comprise an SV40-TAg gene, a TERT gene, a BMI1 gene, a CCND1 gene, a transcript of any of these, an exon of any of these, or any combination thereof. In some embodiments, an exogenous polynucleotide can code for an SV40 large T antigen (SV40-TAg) protein, a biologically active fragment thereof, or any combination thereof. In some embodiments, an exogenous polynucleotide can code for an TERT protein, a biologically active fragment thereof, or any combination thereof. In some embodiments, an exogenous polynucleotide can code for a Bmi-1 protein, a biologically active fragment thereof, or any combination thereof. In some embodiments, an exogenous polynucleotide can code for a cyclin D1 protein, a biologically active fragment thereof, or any combination thereof. In some embodiments, a tumor suppressor protein or biologically active fragment thereof can be a cyclin dependent kinase 4, a retinoblastoma, a p53, a biologically active fragment of any of these, or any combination thereof. In some embodiments, after transfection or transduction, a cell growth cycle of a transfected or transduced isolated cell can be at least partially uninhibited. In some embodiments, after transfection or transduction, a transfected or transduced isolated cell can be grown past about 50 cell divisions, about 70 cell divisions, about 90 cell divisions or about 100 cell divisions. In some embodiments, a medium can comprise a growth medium, a tissue formation medium, or a combination thereof. In some embodiments, after transfection or transduction with an exogenous polynucleotide, a transfected or transduced isolated cell (a) proliferates, (b) avoids senescence, or (c) both, when present in a growth medium. In some embodiments, prior to a seeding, a transfected or transduced isolated cell can be expanded in a growth medium to form a plurality of transfected or transduced cells. In some embodiments, prior to a seeding a transfected or transduced isolated cell can be expanded in a container that at least partially inhibits cellular adherence. In some embodiments, after a seeding, a transfected or transduced isolated cell can be contacted with a tissue formation medium. In some embodiments, a growth medium can comprise a growth factor, a buffer, a salt, a sugar, an amino acid, a lipid, a vitamin, an ECM protein, a fragment of any of these, or any combination thereof. Disclosed herein in some embodiments is an isolated engineered fibroblast or fibroblast-like cell comprising an exogenous molecule, wherein an exogenous molecule at least partially alters an activity of pRB or P53, wherein an isolated engineered fibroblast or fibroblast-like cell can comprise an isolated immortalized bovine fibroblast or fibroblast-like cell as disclosed herein. In some embodiments, an isolated engineered fibroblast or fibroblast-like cell, wherein a cell can be in contact with a scaffold. In some embodiments, a scaffold can comprise a silk fibroin, a cellulose, a cotton, an acetate, an acrylic, a latex fiber, a linen, a nylon, a rayon, a velvet, a modacrylic, an olefin polyester, a polylactic acid (PLA), a saran, a vinyon, a wool, a jute, a hemp, a bamboo, a flax or a combination thereof.

Disclosed herein in some embodiments is a composition comprising: (i) a transfected or transduced isolated fibroblast or fibroblast-like cell comprising an exogenous polynucleotide wherein after transfection or transduction an activity of at least one of pRB or P53 can be at least partially altered in a transfected or transduced isolated fibroblast or fibroblast-like cell, (ii) a scaffold, and (iii) a medium, wherein a transfected or transduced isolated fibroblast or fibroblast-like cell can be at least partially contained on, in, or around a scaffold. In some embodiments, a transfected or transduced isolated fibroblast or fibroblast-like cell can be in contact with a scaffold. In some embodiments, a scaffold can comprise a silk fibroin, a cellulose, a cotton, an acetate, an acrylic, a latex fiber, a linen, a nylon, a rayon, a velvet, a modacrylic, an olefin polyester, a polylactic acid (PLA), a saran, a vinyon, a wool, a jute, a hemp, a bamboo, a flax, or any combination thereof.

Disclosed herein in some embodiments, is an artificial dermal layer comprising: (i) a transfected or transduced isolated fibroblast or fibroblast-like cell comprising an exogenous polynucleotide and (ii) a scaffold, wherein after transfection or transduction an activity of at least one of pRB or P53 can be at least partially altered in a transfected or transduced isolated fibroblast or fibroblast-like cell, and wherein a transfected or transduced isolated fibroblast or fibroblast-like cell can be at least partially contained on, in, or around a scaffold. Disclosed herein in some embodiments, is a method comprising at least partially decellularizing an artificial dermal layer as disclosed herein, to create an at least partially decellularized artificial dermal layer. Disclosed herein in some embodiments, is a method comprising tanning an at least partially decellularized artificial dermal layer as disclosed herein to form a synthetic leather. Disclosed herein in some embodiments, is an artificial dermal layer wherein a scaffold can comprise a silk fibroin, a cellulose, a cotton, an acetate, an acrylic, a latex fiber, a linen, a nylon, a rayon, a velvet, a modacrylic, an olefin polyester, a polylactic acid (PLA), a saran, a vinyon, a wool, a jute, a hemp, a bamboo, a flax, or any combination thereof.

Disclosed herein in some embodiments, is a composition comprising an isolated immortalized fibroblast or fibroblast-like cell and a medium comprising an effective amount of FBS, L-Ascorbic acid 2-phosphate (AA2P) or a salt thereof, Transforming Growth Factor Beta 1 (TGFB1) or a biologically active fragment thereof, or any combination thereof, wherein an effective amount can be sufficient to induce a reporter cell comprising a transfected or transduced polynucleotide to: increase (i) production of collagen; (ii) secretion of collagen; or (iii) both, and arrest cell growth in a reporter cell, when a reporter cell can be present in a medium, relative to an otherwise comparable medium lacking an effective amount of an FBS, an L-Ascorbic acid 2-phosphate (AA2P) or a salt thereof, a Transforming Growth Factor Beta 1 (TGFB1), a biologically active fragment thereof, or a combination, as determined by: transfecting or transducing a cell with a polynucleotide coding for SV40 large T antigen, a biologically active fragment thereof, TERT, a biologically active fragment thereof, or any combination thereof; growing a cell in a medium and an otherwise comparable medium; comparing a growth rate of cells grown in a medium relative to an otherwise comparable medium; and comparing production of collagen produced in a medium relative to an otherwise comparable medium. In some embodiments, a cell can be in contact with a scaffold. In some embodiments, a transfected or transduced isolated fibroblast or fibroblast-like cell as disclosed herein, wherein a scaffold can comprise a silk fibroin, a cellulose, a cotton, an acetate, an acrylic, a latex fiber, a linen, a nylon, a rayon, a velvet, a modacrylic, an olefin polyester, a polylactic acid (PLA), a saran, a vinyon, a wool, a jute, a hemp, a bamboo, a flax, or any combination thereof.

Disclosed herein in some embodiments, is a method comprising: 1) seeding an isolated immortalized animal fibroblast or fibroblast-like cell onto a scaffold to form an artificial dermal layer; 2) at least partially decellularizing an artificial dermal layer to form an at least partially decellularized artificial dermal layer; and 3) tanning an at least partially decellularized artificial dermal layer to form a synthetic leather. In some embodiments, prior to a seeding an isolated immortalized animal fibroblast or fibroblast-like cell can be expanded in culture to form a plurality of isolated immortalized animal fibroblast or fibroblast-like cells. In some embodiments, a plurality of isolated immortalized animal fibroblast or fibroblast-like cells can be grown past a Hayflick limit. In some embodiments, a plurality of isolated immortalized animal fibroblast or fibroblast-like cells can be grown past about 40 cell divisions, about 50 cell divisions, or about 60 cell divisions. In some embodiments, after an expanding, a plurality of isolated immortalized animal fibroblast or fibroblast-like cells can be stored at a temperature below 0° C. In some embodiments, after a storage, a plurality of isolated immortalized animal fibroblast or fibroblast-like cells can be grown in culture before a seeding. In some embodiments, a scaffold can comprise a silk fibroin, a cellulose, a cotton, an acetate, an acrylic, a latex fiber, a linen, a nylon, a rayon, a velvet, a modacrylic, an olefin polyester, a polylactic acid (PLA), a saran, a vinyon, a wool, a jute, a hemp, a bamboo, a flax, or any combination thereof. In some embodiments, a tanning can comprise a cross-linking of a collagen in an artificial dermal layer. In some embodiments, a seeding of an isolated immortalized animal fibroblast or fibroblast-like cell onto a scaffold, a method further can comprise culturing an isolated immortalized animal fibroblast or fibroblast-like cell on a scaffold to form an artificial dermal layer. In some embodiments, at least partially decellularizing can comprise contacting an artificial dermal layer with a salt solution, a crystalline salt, or a combination thereof. In some embodiments, contacting can comprise immersing an artificial dermal layer in a salt solution. In some embodiments, a salt solution can comprise sodium chloride. In some embodiments, a sodium chloride solution can comprise about 30-40% sodium chloride. In some embodiments, an animal fibroblast or fibroblast-like cell can comprise a bovine fibroblast or fibroblast-like cell. In some embodiments, a tanning can comprise a vegetable tanning, a chrome tanning, an aldehyde tanning, a syntan tanning, a bacterial dyeing, or any combination thereof.

Disclosed herein in some embodiments, is a method comprising: 1) seeding an isolated immortalized animal fibroblast or fibroblast-like cell onto a scaffold to form an artificial dermal layer; 2) removing at least a portion of a cell layer from said artificial dermal layer; and 3) tanning an artificial dermal layer to form a synthetic leather. In some embodiments, prior to a seeding an isolated immortalized animal fibroblast or fibroblast-like cell can be expanded in culture to form a plurality of immortalized animal fibroblast or fibroblast-like cells. In some embodiments, a plurality of immortalized animal fibroblast or fibroblast-like cells can be grown past a Hayflick limit. In some embodiments, a plurality of immortalized animal fibroblast or fibroblast-like cells can be grown past about 40 cell divisions, about 50 cell divisions, or about 60 cell divisions. In some embodiments, after an expanding, a plurality of immortalized animal fibroblast or fibroblast-like cells can be stored at a temperature below 0° C. In some embodiments, after a storage, a plurality of immortalized animal fibroblast or fibroblast-like cells can be grown in culture before a seeding onto a scaffold. In some embodiments, culturing an isolated immortalized animal fibroblast or fibroblast-like cell can comprise expanding an immortalized animal fibroblast or fibroblast-like cells. In some embodiments, a tanning can comprise a cross-linking of a collagen in an artificial dermal layer. In some embodiments, an animal fibroblast or fibroblast-like cell can comprise a bovine fibroblast or fibroblast-like cell.

Disclosed herein in some embodiments, is a method comprising: seeding an isolated cell onto a scaffold, wherein an isolated cell can comprise an exogenous molecule; wherein an exogenous molecule can cause anchorage independent or at least partially anchorage independent proliferation based on a direct or indirect stimulus. In some embodiments, a cell can comprise an immortalized cell. In some embodiments, an immortalized cell can comprise an immortalized fibroblast or fibroblast-like cell. In some embodiments, an immortalized fibroblast or fibroblast-like cell can comprise an immortalized bovine fibroblast or fibroblast-like cell. In some embodiments, a molecule can comprise an RNA, a DNA, or a protein. In some embodiments, a DNA codes for a protein.

Disclosed herein in some embodiments, is a method comprising: seeding an isolated cell onto a scaffold, wherein a cell can comprise an at least partially reversible exogenous molecular switch, an exogenous polynucleotide encoding an at least partially reversible exogenous molecular switch, or both. In some embodiments, an isolated cell can comprise an isolated immortalized cell.

Disclosed herein in some embodiments, is a method comprising: transforming an isolated fibroblast cell into an isolated immortalized fibroblast or fibroblast-like cell; introducing into an isolated immortalized fibroblast or fibroblast-like cell an at least partially reversible exogenous molecular switch, an exogenous polynucleotide encoding an at least partially reversible exogenous molecular switch, or both, wherein each of an at least partially reversible exogenous molecular switch an exogenous polynucleotide encoding an at least partially reversible exogenous molecular switch, or both, causes an immortalized fibroblast or fibroblast-like cell to proliferate at least partially anchorage independent or at least partially anchorage dependent based on a direct or indirect stimulus; and proliferating an immortalized fibroblast or fibroblast-like cell anchorage independently.

Disclosed herein in some embodiments, is an engineered cell comprising an at least partially reversible exogenous molecular switch or a polynucleotide encoding an at least partially reversible exogenous molecular switch or a combination thereof, wherein an at least partially reversible exogenous molecular switch causes an engineered cell to grow at least partially anchorage independent or at least partially anchorage dependent based on a direct or indirect stimulus.

Disclosed herein in some embodiments, is a composition comprising: an isolated artificial dermal layer comprising an immortalized fibroblast or fibroblast-like cell cultured in vitro, and a scaffold, wherein an immortalized fibroblast or fibroblast-like cell can be at least partially in contact with a scaffold. In some embodiments, at least a portion of a cell layer of an isolated artificial dermal layer has been removed. In some embodiments, a scaffold can comprise a natural scaffold, a synthetic scaffold or a combination thereof. In some embodiments, a scaffold can comprise a natural scaffold. In some embodiments, a scaffold can comprise a synthetic scaffold. In some embodiments, a scaffold can comprise an at least partially hollow structure. In some embodiments, a scaffold can comprise a collagen, a cellulose, a cotton, an acetate, an acrylic, a latex, a linen, a nylon, a rayon, a velvet, a modacrylic, a saran, a vinyon, a wool, a jute, a hemp, a bamboo, a flax, an alginate, a fibronectin, a poly-p-phenyleneterephthalamide, a polyethylene, a polypropylene, a carrageenan, an agarose, a fibrin, a glass, a silica, an aramid, a carbon, a poly(tetrafluoroethylene), a polyvinyl chloride, a polyvinylidene chloride, a polyvinyl alcohol, a polyacrylonitrile, a chitosan, a polyurethane, a poly(urethane-urea), a polyethylene phthalate, a chitin, a elastin, a keratin, a polyhydroxyalkanoate, a dextran, a pullane, a polyhyaluronic acid, a poly(3-hydroxyalkanoate), a poly(3-hydroxyoctanoate), a poly(3-hydroxyfatty acid), a poly(caprolactone), a poly(para-dioxanone), a laminin, a zein, a casein, a gelatin, a gluten, albumen, a poly L-lactic acid (PLA), a polyglycolic acid (PGA), or any combination thereof. In some embodiments, an immortalized fibroblast or fibroblast-like cell expresses CD10, CD73, CD44, CD90, type I collagen, type III collagen, prolyl-4-hydroxylase beta, or a combination thereof. In some embodiments, an artificial dermal layer can comprise a collagen. In some embodiments, a collagen can be produced at least in part by a collagen producing cell, can be separately added, or any combination thereof.

Disclosed herein in some embodiments is a method comprising: seeding a transfected or transduced isolated cell onto a scaffold to form an artificial dermal layer. In some embodiments a transfected or transduced isolated cell can comprise an exogenous polynucleotide. In some embodiments, a exogenous polynucleotide can encode: (i) a polypeptide which interacts with a tumor suppressor protein or fragment thereof and alters an activity of a tumor suppressor protein or fragment thereof, wherein an activity of a tumor suppressor protein or fragment thereof can be measured by an in vitro assay, or (ii) a polynucleotide that encodes a polypeptide which interacts with a tumor suppressor protein or fragment thereof. In some embodiments, a transfected or transduced cell can be contacted with a medium. In some embodiments, a transfected or transduced cell when in contact with a medium can have: (i) increased collagen production, relative to an otherwise identical cell that is not contacted with a medium, (ii) at least partially increased differentiation, relative to an otherwise identical cell that is not contacted with a medium, or (iii) any combination of (i) and (ii). In some embodiments, a method can further comprise at least partially decellularizing an artificial dermal layer to form an at least partially decellularized dermal layer. In some embodiments, a method can further comprise tanning an at least partially decellularized artificial dermal layer to form a synthetic leather. In some embodiments, an exogenous polynucleotide can code for an SV40 large T antigen (SV40-TAg) polypeptide, a telomerase (TERT) protein, a Bmi-1 protein, a cyclin D1 protein, a biologically active fragment of any of these, or any combination thereof. In some embodiments, an exogenous polynucleotide can comprise an SV40-TAg gene, a TERT gene, a BMI1 gene, a CCND1 gene, a transcript of any of these, an exon of any of these, or any combination thereof. In some embodiments, an exogenous polynucleotide can code for an SV40 large T antigen (SV40-TAg) protein, a biologically active fragment thereof, or any combination thereof. In some embodiments, an exogenous polynucleotide can code for an hTERT protein, a biologically active fragment thereof, or any combination thereof. In some embodiments, an exogenous polynucleotide can code for a Bmi-1 protein, a biologically active fragment thereof, or any combination thereof. In some embodiments, an exogenous polynucleotide can code for a cyclin D1 protein, a biologically active fragment thereof, or any combination thereof. In some embodiments, a tumor suppressor protein or a biologically active fragment thereof can comprise a cyclin dependent kinase 4, a retinoblastoma, a p53, a biologically active fragment of any of these, or any combination thereof. In some embodiments, after transfection or transduction, a cell growth cycle of an isolated cell can be at least partially uninhibited. In some embodiments, after transfection or transduction an isolated cell can be grown past about 50 cell divisions. In some embodiments, after transfection or transduction an isolated cell can be grown past about 70 cell divisions. In some embodiments, after transfection or transduction an isolated cell can be grown past about 90 cell divisions. In some embodiments, after transfection or transduction an isolated cell can be grown past about 100 cell divisions. In some embodiments, after transfection or transduction with an exogenous polynucleotide, an isolated cell (a) can proliferate, (b) can avoid senescence, or (c) both, when an isolated cell can be present in a growth medium. In some embodiments, prior to a seeding, a transfected or transduced isolated cell can be expanded in a growth medium to form a plurality of transfected or transduced cells. In some embodiments, prior to a seeding, a transfected or transduced isolated cell can be expanded in a container that at least partially inhibits cellular adherence. In some embodiments, a medium can comprise a growth medium, a tissue formation medium, or a combination thereof. In some embodiments, after a seeding, a transfected or transduced isolated cell can be contacted with a tissue formation medium. In some embodiments, a growth medium can comprise a growth factor, a buffer, a salt, a sugar, an amino acid, a lipid, a mineral, an ECM protein, or a combination thereof. In some embodiments, a salt can comprise an inorganic salt. In some embodiments, an inorganic salt can comprise about 0.2 g/L Calcium Chloride, about 0.0001 g/L Ferric Nitrate·9H2O, about 0.09767 g/L Magnesium Sulfate (anhydrous), about 0.4 g/L Potassium Chloride, about 3.7 g/L Sodium Bicarbonate, about 6.4 g/L Sodium Chloride, about 0.109 g/L Sodium Phosphate Monobasic (anhydrous), or any combination thereof. In some embodiments, an amino acid can comprise about 0.084 g/L L-Arginine·HCl, about 0.0626 g/L L-Cystine·2HCl, about 0.03 g/L Glycine, about 0.042 g/L L-Histidine·HCl·H2O, about 0.105 g/L L-Isoleucine, about 0.105 g/L L-Leucine, about 1.46 g/L L-Lysine HCl, about 0.03 g/L L-Methionine, about 0.066 g/L L-Phenylalanine, about 0.042 g/L L-Serine, about 0.095 g/L L-Threonine, about 0.016 g/L L-Tryptophan, about 0.12037 g/L L-Tyrosine·2Na·2H2O, about 0.094 g/L L-Valine, about 0.584 g/L L-Glutamine, a stereoisomer of any of these, a salt of any of these, or any combination thereof. In some embodiments, a vitamin can comprise about 0.004 g/L Choline Chloride, about 0.004 g/L Folic Acid, about 0.0072 g/L myo-Inositol, about 0.004 g/L Niacinamide, about 0.004 g/L D-Pantothenic Acid (hemicalcium), about 0 g/L Pyridoxal·HCl, about 0.004 g/L Pyridoxine·HCl, about 0.0004 g/L Riboflavin, about 0.004 g/L Thiamine·HCl, a stereoisomer of any of these, a salt of any of these, or any combination thereof. In some embodiments, a sugar can comprise D-Glucose, a stereoisomer thereof, a salt thereof, or any combination thereof. In some embodiments, a pH indicator can comprise about 0.0159 g/L Phenol Red·Na, about 0.11 g/L Pyruvic Acid·Na, a stereoisomer of any of these, a salt of any of these, or any combination thereof. In some embodiments, a growth medium can comprise an amino acid, a vitamin, an inorganic salt, a fetal bovine serum, an antibiotic, an antimycotic, or any combination thereof. In some embodiments, a tissue formation medium can comprise a growth factor, a buffer, a salt, a sugar, an amino acid, a lipid, a mineral, an ECM protein, a human platelet lysate, an acid citrate dextrose, a heparin, an ascorbic acid, a TGF-β1, a normocin, a serum, a serum alternative, a non-essential amino acid, an antibiotic, an antimycotic, or any combination thereof. In some embodiments, a tissue formation medium further can comprise from about 0.10% to about 40% of a serum, a serum alternative, or a combination thereof. In some embodiments, an amino acid can comprise a Glycine, an Alanine, an L-Arginine hydrochloride, an L-Asparagine-H2O, an L-Aspartic acid, an L-Cysteine hydrochloride-H2O, an L-Cystine 2HCl, an L-Glutamic Acid, an L-Glutamine, an L-Histidine hydrochloride-H2O, an L-Isoleucine, an L-Leucine, an L-Lysine hydrochloride, an L-Methionine, an L-Phenylalanine, an L-Proline, an L-Serine, an L-Threonine, an L-Tryptophan, an L-Tyrosine disodium salt dihydrate, an L-Valine, a stereoisomer of any of these, a salt of any of these, or any combination thereof. In some embodiments, a vitamin can comprise a biotin, a choline chloride, a D-Calcium pantothenate, a Folic acid, a Niacinamide, a Pyridoxine hydrochloride, a Riboflavin, a Thiamine hydrochloride, a Vitamin B12, an i-Inositol, a stereoisomer of any of these, a salt of any of these, or any combination thereof. In some embodiments, an inorganic salt can comprise a calcium chloride, a cupric sulfate, a ferric nitrate, a ferric sulfate, a magnesium chloride, a magnesium sulfate, a potassium chloride, a sodium bicarbonate, a sodium chloride, a sodium phosphate dibasic, a sodium phosphate monobasic, a zinc sulfate, a stereoisomer of any of these, a salt of any of these, or any combination thereof. In some embodiments, a medium further can comprise a D-Glucose (Dextrose), a hypoxanthine Na, a linoleic acid, a lipoic acid, a phenol red, a putrescine 2HCl, a Sodium pyruvate, a thymidine, a stereoisomer of any of these, a salt of any of these, or any combination thereof. In some embodiments, a serum alternative can contain substantially no animal derived products, can be xeno-free, or a combination thereof. In some embodiments, a serum alternative can comprise a growth factor, an insulin transferrin, a cytokine, an essential amino acid, a nonessential amino acid, a protein, an extracellular matrix protein (ECM), a laminin, a fibronectin, a vitronectin, a cell adhesion peptide, an RGD, an extracellular matrix fragment, a hormone, a collagen, an albumin, a lipid, a glycoprotein, a protein fragment, or any combination thereof. In some embodiments, a serum can comprise a fetal bovine serum (FBS), a horse serum, a fetal calf serum, or any combination thereof. In some embodiments, a medium does not comprise TGF beta. In some embodiments, after a seeding, a transfected or transduced isolated cell can be contacted with a tissue formation medium. In some embodiments, a transfected or transduced isolated cell (a) at least partially increases production of a collagen, (b) has an at least partially arrested cell growth, or (c) both, when present in a tissue formation medium, relative to an otherwise comparable transfected or transduced isolated cell that has not been contacted with a tissue formation medium. In some embodiments, a transfected or transduced isolated cell can be at least partially differentiated when contacted with a tissue formation medium. In some embodiments, after a seeding, a transfected or transduced isolated cell can be contacted with a medium comprising L-ascorbic acid 2-phosphate (AA2P), a salt thereof, a biologically active fragment thereof, or a combination of any of these. In some embodiments, a transfected or transduced isolated cell (a) at least partially increases production of collagen, (b) has an at least partially arrested cell growth, or (c) both, when present in a medium comprising an AA2P, a salt thereof, a TGFB1, a biologically active fragment thereof, or a combination, relative to an otherwise comparable medium lacking an AA2P, a salt thereof, a TGFB1, a biologically active fragment thereof, or a combination. In some embodiments, a transfected or transduced isolated cell can be at least partially differentiated when contacted with a medium. In some embodiments, a transfected or transduced isolated cell can comprise an isolated immortalized cell, an isolated reprogrammed cell, an isolated progenitor cell, an isolated mesenchymal stem cell, or any combination thereof a transfected or transduced isolated cell can comprise an isolated fibroblast cell, an isolated mesenchymal cell, an isolated stem cell derived cell, an isolated umbilical cord stem cell, an isolated amniotic tissue cell, an isolated scar tissue cell, or any combination thereof. In some embodiments, a cell can display markers of collagen production. In some embodiments, a cell can be isolated by flow cytometry. In some embodiments, a transfected or transduced isolated cell can be isolated from a bovine, a non-human mammal, a reptile, a bird, a shark, a kangaroo, a fish, or an eel. In some embodiments, a transfected or transduced isolated cell can be isolated from a bovine, and wherein a transfected or transduced isolated cell can comprise a bovine fibroblast cell. In some embodiments, a transfected or transduced isolated cell can be isolated from a reptile, and wherein a transfected or transduced isolated cell can comprise a turtle cell, a snake cell, a lizard cell, an amphibian cell, a crocodile cell, or an alligator cell. In some embodiments, a transfected or transduced isolated cell can be isolated from a non-human mammal, wherein a transfected or transduced isolated cell can comprise an antelope cell, a bear cell, a beaver cell, a bison cell, a boar cell, a camel cell, a caribou cell, a cat cell, a cattle cell, a deer cell, a dog cell, an elephant cell, an elk cell, a fox cell, a giraffe cell, a goat cell, a hare cell, a horse cell, an ibex cell, a lion cell, a llama cell, a lynx cell, a mink cell, a moose cell, an oxen cell, a peccary cell, a pig cell, a rabbit cell, a rhino cell, a seal cell, a sheep cell, a squirrel cell, a tiger cell, a whale cell, a wolf cell, a yak cell, or a zebra cell. In some embodiments, a transfected or transduced isolated cell can be isolated from a bird, wherein a transfected or transduced isolated cell can comprise a chicken cell, a duck cell, an emu cell, a goose cell, a grouse cell, an ostrich cell, a pheasant cell, a pigeon cell, a quail cell, or a turkey cell. In some embodiments, a cell can comprise an animal cell. In some embodiments, an animal cell can comprise an anserine cell, an aquiline cell, an assinine cell, a bovine cell, a cancrine cell, a canine cell, a cervine cell, a corvine cell, an equine cell, an elapine cell, an elaphine cell, a feline cell, a hircine cell, a leonine cell, a leporine cell, a lupine cell, a murine cell, a pavonine cell, a piscine cell, a porcine cell, a rusine cell, a serpentine cell, an ursine cell, a vulpine cell, a primate cell, an ovine cell, a bird cell, a marsupial cell, a reptile cell, a lagomorph cell, or any combination thereof. In some embodiments, a cell can be derived from a scar tissue, an umbilical cord, or a combination thereof. In some embodiments, prior to a seeding, a transfected or transduced cell can be selected for a presence of an exogenous polynucleotide. In some embodiments, a selection for a presence of an exogenous polynucleotide can comprise an antibiotic selection. In some embodiments, an antibiotic can comprise a puromycin. In some embodiments, a scaffold can comprise a porous material. In some embodiments, a transfected or transduced isolated cell can be engrafted to a scaffold. In some embodiments, a scaffold can comprise a synthetic material, a non-synthetic material, or a combination thereof. In some embodiments, a natural material can comprise a silk, a natural tissue adhesive, a fibrin glue, a collagen, a basement membrane protein, an extracellular matrix, or a combination thereof. In some embodiments, a scaffold can comprise a polyethylene (PE), a polypropylene (PP), a Polyethylene terephthalate (PET), a Polyamide 6,6 (PA 6,6), a Polyamide 11 (PA 11), a Polyvinylidene fluoride (PVDF), a Polyethylene furanoate (PEF), a Polyurethane (PU), a Polyhydroxyalkanoate (PHA), a Polyhydroxybutyrate (PHB), a Poly(3-hydroxybutyrate-co-3-hydroxyvalerate) (PHBV), a Polylactic acid (PLA), a Polycaprolactone (PCL), a Polybutylene succinate (PBS), a Poly(glycolic) acid (PGA), a Poly(lactic-co-glycolic acid (PLGA), a Polyvinyl Alcohol (PVOH), an Alginate, a Copolymer PEGylated fibrin (P-fibrin), a Poly(glycerol sebacate) (PGS), a poly(L-lactic acid) (PLLA), a Poly(lactic-coglycolic acid) (PLGA), a Poly-D,L-lactic acid/polyethylene glycol/poly-D,L-lactic acid (PDLLA-PEG), a hyaluronic acid (HA), a carbon nanotube, a Thermoplastic Starch, a Lyocell/Tencel (Cellulose), a Cotton, a Bast fiber, a Viscose Bamboo, a TiO2 nanofiber, a cellulose material, a hydrogel material, an alginate, a gelatin, a nylon, a polyester, a silk, a material cross-linked with a cell adhesion peptide, a material cross-linked with growth factors, or any combination thereof.

Disclosed herein in some embodiments, is an engineered cell comprising an exogenous molecule. In some embodiments, an exogenous molecule at least partially alters an activity of pRB or P53. In some embodiments, an engineered cell can comprise an immortalized animal cell. In some embodiments, an animal cell can comprise a non-human animal cell. In some embodiments, a non-human animal cell can comprise a bovine cell. In some embodiments, a molecule can comprise an RNA, a DNA, a small molecule, a salt thereof, a polypeptide, a hormone, or a biologically active fragment thereof. In some embodiments, a molecule can comprise a DNA, wherein a DNA can code for a polypeptide or a biologically active fragment thereof. In some embodiments, a molecule can comprise an RNA, wherein an RNA can comprise a mRNA, a siRNA, or a miRNA.

Disclosed herein in some embodiments, is a method comprising contacting a transfected or transduced isolated cell comprising an exogenous polynucleotide with a medium comprising from about 2% to about 40% FBS, wherein a transfected or transduced cell when present in a medium comprising an FBS (a) produces collagen, (b) has an at least partially arrested cell growth, or (c) both, and wherein after transfection or transduction an activity of at least one of pRB or P53 can be at least partially altered in a transfected or transduced isolated cell. In some embodiments, a medium can comprise about 20% FBS. In some embodiments, a medium further can comprise L-ascorbic acid 2-phosphate (AA2P), a salt thereof, transforming growth factor beta 1 (TGFB1), a biologically active fragment thereof, or any combination thereof. In some embodiments, an exogenous polynucleotide can code for an SV40 large T antigen, an hTERT, a Bmi-1, a cyclin D1, a biologically active fragment of any of these, or any combination thereof. In some embodiments, a transfected or transduced isolated cell can be a fibroblast cell. In some embodiments, a transfected or transduced isolated cell can be isolated from a bovine, a non-human mammal, a reptile, a bird, a shark, a kangaroo, a fish, or an eel. In some embodiments, a synthetic leather can be made into a form of any item selected from a group consisting of a bag, a belt, a watch strap, a package, a shoe, a boot, a footwear, a glove, a clothing, a vest, a jacket, a pant, a hat, a shirt, an undergarment, a luggage, a clutch, a purse, a ball, a backpack, a wallet, a saddle, a harness, a chap, a whip, furniture, furniture accessories, an upholstery, an automobile car seat, an automobile interior, and any combination thereof.

Disclosed herein in some embodiments, is a composition comprising: (i) a transfected or transduced isolated cell comprising an exogenous polynucleotide wherein after transfection or transduction an activity of at least one of pRB or P53 can be at least partially altered in a transfected or transduced isolated cell, (ii) a scaffold, and (iii) a medium. In some embodiments, a transfected or transduced isolated cell can be at least partially contained on, in, or around a scaffold.

Disclosed herein in some embodiments, is an artificial dermal layer comprising: (i) a transfected or transduced isolated cell comprising an exogenous polynucleotide and (ii) a scaffold. In some embodiments, after transfection or transduction an activity of at least one of pRB or P53 can be at least partially altered in a transfected or transduced isolated cell. In some embodiments, a transfected or transduced isolated cell can be at least partially contained on, in, or around a scaffold. In some embodiments, at least a portion of a tissue can be at least partially decellularized. In some embodiments, at least a portion of an at least partially decellularized tissue can be tanned to form a synthetic leather.

Disclosed herein in some embodiments, is a composition comprising an immortalized fibroblast cell and a medium comprising an effective amount of: FBS, L-Ascorbic acid 2-phosphate (AA2P) or a salt thereof, Transforming Growth Factor Beta 1 (TGFB1) or a biologically active fragment thereof, or any combination thereof, wherein an effective amount can be sufficient to induce a reporter cell comprising a transfected or transduced polynucleotide to: increase (i) production of collagen; (ii) secretion of collagen; or (iii) both, and arrest cell growth in a reporter cell, when a reporter cell can be present in a medium, relative to an otherwise comparable medium lacking an effective amount of a FBS, a L-Ascorbic acid 2-phosphate (AA2P) or a salt thereof, a Transforming Growth Factor Beta 1 (TGFB1) or a biologically active fragment thereof, or a combination, as determined by: transfecting or transducing a cell with a polynucleotide coding for SV40 large T antigen, a biologically active fragment thereof, hTERT, a biologically active fragment thereof, or any combination thereof, growing a cell in a medium and an otherwise comparable medium; comparing a growth rate of a cells grown in a medium relative to an otherwise comparable medium; and comparing production of collagen produced in a medium relative to an otherwise comparable medium. Disclosed herein in some embodiments, is a composition comprising: (i) a transfected or transduced isolated cell comprising an exogenous polynucleotide wherein after transfection or transduction an activity of at least one of pRB or P53 can be at least partially altered in a transfected or transduced isolated cell and (ii) a medium as disclosed herein. Disclosed herein in some embodiments, is a composition comprising: (i) a transfected or transduced isolated cell comprising an exogenous polynucleotide wherein after transfection or transduction an activity of at least one of pRB or P53 can be at least partially altered in a transfected or transduced isolated cell and (ii) a medium as disclosed herein.

Disclosed herein in some embodiments, are methods comprising seeding an immortalized bovine fibroblast cell onto a scaffold to form an artificial dermal layer. In some embodiments, a method can further comprise at least partially decellularizing an artificial dermal layer to form an at least partially decellularized dermal layer. In some embodiments, a method can further comprise tanning an at least partially decellularized artificial dermal layer to form a synthetic leather. In some embodiments, prior to a seeding an immortalized bovine fibroblast cell can be expanded in culture to form a plurality of immortalized bovine fibroblast cells. In some embodiments, a plurality of immortalized bovine fibroblast cells can be grown past a Hayflick limit. In some embodiments, a plurality of immortalized bovine fibroblast cells can be grown past about 40 cell divisions, about 50 cell divisions, or about 60 cell divisions. In some embodiments, after an expanding, a plurality of immortalized bovine fibroblast cells can be stored at a temperature below 0° C. In some embodiments, after a storage, a plurality of immortalized bovine fibroblast cells can be grown in a culture before a seeding onto a scaffold. In some embodiments, a culturing of an immortalized bovine fibroblast cell can comprise expanding an immortalized bovine fibroblast cell. In some embodiments, after a seeding of an immortalized bovine fibroblast cell onto a scaffold, a method can further comprise culturing an immortalized bovine fibroblast cell on a scaffold to form an artificial dermal layer. In some embodiments, an at least partially decellularizing can comprise contacting an artificial dermal layer with a salt solution. In some embodiments, a contacting with a salt solution can comprise immersing an artificial dermal layer in s salt solution. In some embodiments, a salt can comprise sodium chloride. In some embodiments, a concentration of a sodium chloride can comprise about 30% to about 40%. In some embodiments, a tanning can comprise a cross-linking of a collagen in an artificial dermal layer. In some embodiments, a tanning can comprise treating an isolated artificial dermal layer to produce a leather. In some embodiments, a tanning can produce a synthetic leather that resembles a natural leather. In some embodiments, a tanning can comprise a vegetable tanning, a chrome tanning, an aldehyde tanning, a syntan tanning, a bacterial dyeing, or any combination thereof. In some embodiments, a vegetable tanning can comprise using a tannin. In some embodiments, a chrome tanning can comprise using a chromium salt. In some embodiments, a chromium salt can comprise a chromium sulfate. In some embodiments, an aldehyde can comprise a glutaraldehyde compound, an oxazolidine compound, or any combination thereof. In some embodiments, a syntan can comprise a synthetic tannin, an aromatic polymer, or any combination thereof. In some embodiments, a tanning can be performed to convert a protein in an artificial dermal layer into a stable material that will not putrefy, while allowing a material to remain flexible. In some embodiments, a pH of a cell layer or layered structure can be adjusted. In some embodiments, a lowering of a pH can comprise lowering to about 6, about 5, about 4, about 3, about 2, or about 1. In some embodiments, a lowering of a pH can comprise lowering to about 2.8-3.2. In some embodiments, after a lowering of a pH, a pH can then be raised. In some embodiments, a raising of a pH can comprise raising to about 3.8-4.2. .

Disclosed herein in some embodiments, are methods comprising seeding an immortalized bovine fibroblast cell onto a scaffold to form an artificial dermal layer. In some embodiments, a method can further comprise tanning an artificial dermal layer to form a synthetic leather. In some embodiments, prior to a seeding, an immortalized bovine fibroblast cell can be expanded in culture to form a plurality of immortalized bovine fibroblast cells. In some embodiments, a plurality of immortalized bovine fibroblast cells can be grown past a Hayflick limit. In some embodiments, a plurality of immortalized bovine fibroblast cells can be grown past about 40 cell divisions, about 50 cell divisions, or about 60 cell divisions. In some embodiments, after an expanding, a plurality of immortalized bovine fibroblast cells can be stored at a temperature below 0° C. In some embodiments, after a storage, a plurality of immortalized bovine fibroblast cells can be grown in a culture before a seeding onto a scaffold. In some embodiments, a culturing of an immortalized bovine fibroblast cell can comprise expanding an immortalized bovine fibroblast cell. In some embodiments, after a seeding of an immortalized bovine fibroblast cell onto a scaffold, a method can further comprise culturing an immortalized bovine fibroblast cell on a scaffold to form an artificial dermal layer. In some embodiments, a tanning can comprise a cross-linking of a collagen in an artificial dermal layer. In some embodiments, a tanning can comprise a cross-linking of a collagen in an artificial dermal layer. In some embodiments, a tanning can comprise treating an isolated artificial dermal layer to produce a leather. In some embodiments, a tanning can produce a synthetic leather that resembles a natural leather. In some embodiments, a tanning can comprise a vegetable tanning, a chrome tanning, an aldehyde tanning, a syntan tanning, a bacterial dyeing, or any combination thereof. In some embodiments, a vegetable tanning can comprise using a tannin. In some embodiments, a chrome tanning can comprise using a chromium salt. In some embodiments, a chromium salt can comprise a chromium sulfate. In some embodiments, an aldehyde can comprise a glutaraldehyde compound, an oxazolidine compound, or any combination thereof. In some embodiments, a syntan can comprise a synthetic tannin, an aromatic polymer, or any combination thereof. In some embodiments, a tanning can be performed to convert a protein in an artificial dermal layer into a stable material that will not putrefy, while allowing a material to remain flexible. In some embodiments, a pH of a cell layer or layered structure can be adjusted. In some embodiments, a lowering of a pH can comprise lowering to about 6, about 5, about 4, about 3, about 2, or about 1. In some embodiments, a lowering of a pH can comprise lowering to about 2.8-3.2. In some embodiments, after a lowering of a pH, a pH can then be raised. In some embodiments, a raising of a pH can comprise raising to about 3.8-4.2.

Disclosed herein in some embodiments, are methods comprising seeding a cell onto a scaffold. In some embodiments, a cell can comprise an exogenous molecule. In some embodiments, an exogenous molecule can cause anchorage independent or at least partially anchorage independent proliferation based on a direct or indirect stimulus. In some embodiments, a cell can comprise an immortalized cell. In some embodiments, an immortalized cell can comprise an immortalized fibroblast cell. In some embodiments, an immortalized fibroblast cell can comprise an immortalized bovine fibroblast cell. In some embodiments, a molecule can comprise an RNA, a DNA, or a protein.

Disclosed herein in some embodiments, are methods comprising seeding cells onto a scaffold. In some embodiments, a cell can comprise an at least partially reversible exogenous molecular switch, an exogenous polynucleotide encoding an at least partially reversible exogenous molecular switch, or both. In some embodiments, a cell can be an immortalized cell. In some embodiments, a method can further comprise before a seeding, proliferating cells in one or more environments, for example a first environment. In some embodiments, a cell can comprise an at least partially reversible exogenous molecular switch, an exogenous polynucleotide encoding an at least partially reversible exogenous molecular switch, or both. In some embodiments, a cell can be a plurality of cells. In some embodiments, a plurality of cells can comprise a plurality of an at least partially reversible exogenous molecular switch, an exogenous polynucleotide encoding an at least partially reversible exogenous molecular switch, or both. In some embodiments, a method can comprise a plurality of cells. In some embodiments, a plurality of an at least partially reversible exogenous molecular switch, an exogenous polynucleotide encoding an at least partially reversible exogenous molecular switch, or both can be at least partially on during proliferating and at least partially off during seeding. In some embodiments, a plurality of an at least partially reversible exogenous molecular switch, an exogenous polynucleotide encoding an at least partially reversible exogenous molecular switch, or both can be at least partially on during proliferating and at least partially off during seeding. In some embodiments, an at least partially reversible exogenous molecular switch individually can be at least partially on during proliferating and at least partially off during seeding. In some embodiments, an at least partially reversible exogenous molecular switch individually can be at least partially off during proliferating and at least partially on during seeding. In some embodiments, an at least partially reversible exogenous molecular switch can be at least partially on during proliferating. In some embodiments, an at least partially reversible exogenous molecular switch can be at least partially off during proliferating. In some embodiments, a cell can be a eukaryotic cell. In some embodiments, a cell can be selected from a group consisting of a primate cell, bovine cell, ovine cell, porcine cell, equine cell, canine cell, feline cell, rodent cell, bird cell, marsupial, reptile, and lagomorph animal cell. In some embodiments, a first environment can be in suspension. In some embodiments, an at least partially reversible exogenous molecular switch at least partially can cause anchorage independent or at least partially anchorage dependent proliferation based on a direct or indirect stimulus. In some embodiments, at least partially anchorage independent proliferation can be at least partially a result of an increase in expression of: integrin-linked kinase (ILK), cyclin D1, Cdk4, ST6 N-Acetylgalactosaminide Alpha-2, 6-Sialytransferase 5 (ST6GALNAC5), or a combination thereof. In some embodiments, an at least partially reversible exogenous molecular switch can be at least partially on during proliferating and at least partially off during seeding. In some embodiments, an at least partially reversible exogenous molecular switch can be at least partially off during proliferating and at least partially on during seeding. In some embodiments, a presence of a stimuli can cause an increased expression or a decreased expression of an exogenous polynucleotide encoding an at least partially reversible exogenous molecular switch. In some embodiments, an absence of a stimuli can cause an increased expression or a decreased expression of an exogenous polynucleotide encoding an at least partially reversible exogenous molecular switch. In some embodiments, a proliferating can be in a presence of a stimulus. In some embodiments, proliferating can be in an absence of a stimulus. In some embodiments, a stimulus can be selected from a group consisting of a change in: pH, light, temperature, an electric current, microenvironment, presence or absence of ions, level of an ion, presence of one or more ion type, a change in a mechanical stimulus, a change in a culture medium composition and any combination thereof. In some embodiments, a stimulus can comprise a change in a temperature. In some embodiments, an at least partially reversible exogenous molecular switch can be reversible based on a temperature. In some embodiments, a cell can be exposed to a temperature of from about 28° C. to about 34° C. during proliferating. In some embodiments, a cell can be exposed to a temperature from about 37° C. to about 41° C. during or following seeding. In some embodiments, a scaffold can be at least partially natural or synthetic. In some embodiments, a scaffold can comprise at least one of a group consisting of a silk, a polylactide, a polyglycolide, a polyester, a polycaprolactone, a chitosan, a hydrogel and a combination thereof. In some embodiments, a cell can produce an extracellular matrix protein. In some embodiments, an extracellular matrix protein can be selected from a group consisting of collagen type I, collagen type III, elastin, fibronectin, laminin, and a combination thereof. In some embodiments, an extracellular matrix protein can comprise collage. In some embodiments, a method can further comprise generating at least a portion of a synthetic leather comprising a cell or a portion of tissue developed therefrom. In some embodiments, a synthetic leather can comprise at least a portion of a tissue. In some embodiments, a tissue can comprise collagen type I. In some embodiments, a method can further comprise before a seeding, proliferating a cell in a first environment and then directly or indirectly adding an at least partially reversible exogenous molecular switch to a proliferating cell. In some embodiments, a cell can be seeded at a density of about 50,000 cells/cm² to about 1,000,000 cells/cm². In some embodiments, seeding a cell onto a scaffold can comprise seeding one side of a scaffold. In some embodiments, a second side of a scaffold can be seeded without flipping a scaffold. In some embodiments, seeding a cell onto a scaffold can comprise seeding on more than one side of a scaffold. In some embodiments, seeding can be consecutively or concurrently. In some embodiments, seeding can comprise seeding on one side of a scaffold then flipping a scaffold over and seeding another side of a scaffold. In some embodiments, a cell or an immortalized cell can be modified to have enhanced extracellular matrix production as compared to an otherwise comparable wildtype cell that may not comprise an at least partially reversible exogenous molecular switch or a polynucleotide encoding an at least partially reversible exogenous molecular switch. In some embodiments, an extracellular matrix can comprise collagen type I, collagen type III, elastin, fibronectin, laminin, or a combination thereof. In some embodiments, a method can further comprise engineering a tissue comprising a cell or an immortalized cell. In some embodiments, a method can further comprise tanning a tissue. In some embodiments, an extracellular matrix can comprise collagen type I.

Also disclosed herein, are methods comprising transforming a cell into an immortalized cell. In some embodiments, a method can comprise introducing into an immortalized cell an at least partially reversible molecular switch, an exogenous polynucleotide encoding an at least partially reversible exogenous molecular switch, or both. In some embodiments, each of an at least partially reversible molecular switch, an exogenous polynucleotide encoding an at least partially reversible exogenous molecular switch, or both, can cause an immortalized cell to proliferate at least partially anchorage independent or at least partially anchorage dependent based on a direct or indirect stimulus. In some embodiments, a method can comprise proliferating an immortalized cell anchorage independently. In some embodiments, a cell can be selected from a group consisting of a primate cell, bovine cell, ovine cell, porcine cell, equine cell, canine cell, feline cell, rodent cell, bird cell, marsupial, reptile, and lagomorph animal cell. In some embodiments, a cell can be a bovine cell. In some embodiments, a cell can be a stem cell. In some embodiments, a stem cell can be selected from a group consisting of a mesenchymal stem cell, pluripotent stem cell, induced pluripotent stem cell and an embryonic stem cell. In some embodiments, a cell can be selected from a group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof. In some embodiments, a cell can be a fibroblast or a fibroblast-like cell. In some embodiments, transforming can comprise increasing or decreasing expression of an oncogene or genes involved in regulation of cells proliferation. In some embodiments, a cell can express a polypeptide or a biologically active fragment thereof encoded by: TERT, Bmi1, or any combination thereof. In some embodiments, an immortalized cell can express a polypeptide or a biologically active fragment thereof encoded by: TERT, CcnD1, Cdk4, or any combination thereof. In some embodiments, a method can further comprise exposing an immortalized cell to a stimulus. In some embodiments, a stimulus can be selected from a group consisting of a change in: pH, light, temperature, an electric current, microenvironment, presence or absence of ions, level of an ion, presence of one or more ion type, a change in a mechanical stimulus, a change in a culture medium composition and any combination thereof. In some embodiments, a stimulus can comprise a change in a temperature. In some embodiments, a temperature can be from about 28° C. to about 34° C. In some embodiments, a stimulus can cause an immortalized cell to proliferate at least partially anchorage independent. In some embodiments, anchorage independent can comprise proliferation in suspension. In some embodiments, at least partially anchorage independent proliferation can be at least partially a result of an increase in expression of: integrin-linked kinase (ILK), cyclin D1, Cdk4, ST6 N-Acetylgalactosaminide Alpha-2, 6-Sialytransferase 5 (ST6GALNAC5), or a combination thereof. In some embodiments, removal of a stimulus can cause an immortalized cell to proliferate at least partially anchorage dependent. In some embodiments, a method can further comprise exposing an immortalized cell to a second stimulus. In some embodiments, a second stimulus can be selected from a group consisting of a change in: pH, light, temperature, an electric current, microenvironment, a presence or absence of ions, a level of an ion, presence of one or more ion type, a change in a mechanical stimulus, a change in a culture medium composition and any combination thereof. In some embodiments, a second stimulus can comprise a change in a temperature. In some embodiments, a temperature can be from about 37° C. to about 41° C. In some embodiments, a second stimulus can cause an immortalized cell to proliferate at least partially anchorage dependent. In some embodiments, a removal of a second stimulus can cause an immortalized cell to proliferate at least partially anchorage independent. In some embodiments, anchorage dependent can comprise proliferation on a substrate. In some embodiments, a method can further comprise seeding an immortalized cell on a substrate. In some embodiments, an immortalized cell can be seeded at a density from about 50,000 cells/cm² to about 1,000,000 cells/cm². In some embodiments, seeding an immortalized cell on a substrate can comprise seeding one side of a substrate. In some embodiments, a second side of a substrate can be seeded without flipping a substrate. In some embodiments, seeding an immortalized cell on a substrate can comprise seeding on more than one side of a substrate. In some embodiments, seeding can be consecutively or concurrently. In some embodiments, seeding can comprise seeding on one side of a substrate then flipping a substrate over and seeding another side of a substrate. In some embodiments, anchorage dependent proliferation can be at least partially on, in, or around a substrate. In some embodiments, a substrate can comprise a scaffold. In some embodiments, a scaffold can be at least partially natural or synthetic. In some embodiments, a scaffold can comprise a silk, a polylactide, a polyglycolide, a polyester, a polycaprolactone, a chitosan, a hydrogel or a combination thereof. In some embodiments, a cell or an immortalized cell can be modified to have enhanced extracellular matrix production as compared to an otherwise comparable wildtype cell that may not comprise an at least partially reversible exogenous molecular switch or a polynucleotide encoding an at least partially reversible exogenous molecular switch. In some embodiments, an extracellular matrix can comprise collagen type I, collagen type III, elastin, fibronectin, laminin, or a combination thereof. In some embodiments, an extracellular matrix protein can comprise collagen. In some embodiments, a method can further comprise engineering a tissue comprising a cell or an immortalized cell disclosed herein. In some embodiments, a method can further comprise tanning a tissue comprising an immortalized cell disclosed herein. In some embodiments, an extracellular matrix can comprise collagen type I.

Also disclosed herein, are engineered cells. In some embodiments, an engineered cell can comprise an at least partially reversible exogenous molecular switch or a polynucleotide encoding an at least partially reversible exogenous molecular switch or a combination thereof. In some embodiments, an engineered cell can be an immortalized bovine cell. In some embodiments, an at least partially reversible exogenous molecular switch can cause an engineered cell to grow at least partially anchorage independent or at least partially anchorage dependent based on a direct or indirect stimulus. In some embodiments, proliferation of an engineered cell can be determined at least in part by a method selected from a group consisting of manual cell counting, automated cell counting and indirect cell counting. In some embodiments, an engineered cell can be selected from a group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof. In some embodiments, an engineered cell can be a fibroblast, or a fibroblast-like cell. In some embodiments, an engineered cell can be derived from a group consisting of a pluripotent stem cell, a mesenchymal stem cell, induced pluripotent stem cell and an embryonic stem cell. In some embodiments, an engineered cell can be a cell from a cell line comprising a plurality of cells. In some embodiments, an engineered cell can express an exogenous oncogene or genes involved in regulation of cells proliferation. In some embodiments, an engineered cell can express a polypeptide or a biologically active fragment thereof encoded by: TERT, Bmi1, or any combination thereof. In some embodiments, an engineered cell can express a polypeptide or a biologically active fragment thereof encoded by: TERT, CcnD1, Cdk4, or any combination thereof. In some embodiments, an engineered cell can be modified to have enhanced extracellular matrix production as compared to an otherwise comparable wildtype cell that does not comprise an at least partially reversible exogenous molecular switch or a polynucleotide encoding an at least partially reversible exogenous molecular switch. In some embodiments, an extracellular matrix can comprise collagen type I, collagen type III, elastin, fibronectin, laminin, or a combination thereof. In some embodiments, an extracellular matrix protein can comprise collagen. In some embodiments, an at least partially reversible exogenous molecular switch or a polynucleotide encoding an at least partially reversible exogenous molecular switch can be configured to at least partially increase or decrease expression in response to a stimulus. In some embodiments, increased expression of an at least partially reversible exogenous molecular switch or a polynucleotide encoding an at least partially reversible exogenous molecular switch can cause at least partial anchorage dependent proliferation. In some embodiments, increased expression of an at least partially reversible exogenous molecular switch or a polynucleotide encoding an at least partially reversible exogenous molecular switch can cause at least partial anchorage independent proliferation. In some embodiments, decreased expression of an at least partially reversible exogenous molecular switch or a polynucleotide encoding an at least partially reversible exogenous molecular switch can cause at least partial anchorage dependent proliferation. In some embodiments, decreased expression of an at least partially reversible exogenous molecular switch or a polynucleotide encoding an at least partially reversible exogenous molecular switch can cause at least partial anchorage independent proliferation. In some embodiments, at least partial anchorage dependent proliferation can consume more, same or less nutrients or growth factors as compared to anchorage independent proliferation. In some embodiments, at least partially anchorage independent proliferation can consume more, same, or less nutrients or growth factors as compared to at least partially anchorage dependent proliferation. In some embodiments, at least partially anchorage independent proliferation can be at least partially a result of an increase in expression of an integrin-linked kinase (ILK), a cyclin D1, a Cdk4, an ST6 N-Acetylgalactosaminide Alpha-2, a 6-Sialytransferase 5 (ST6GALNAC5), or a combination thereof. In some embodiments, an at least partially reversible exogenous molecular switch or a polynucleotide encoding an at least partially reversible exogenous molecular switch can be a single switch, or a plurality of switches. In some embodiments, a polynucleotide encoding an at least partially reversible exogenous molecular switch can encode a selectable marker. In some embodiments, a selectable marker can comprise a fluorescent protein. In some embodiments, an engineered cell can comprise a recombinant selectable marker. In some embodiments, a selectable marker can be selected from a group consisting of an antibiotic resistance gene, a gene encoding a fluorescent protein, and a gene expressing an auxotrophic marker. In some embodiments, an engineered cell can comprise a polynucleotide encoding an at least partially reversible exogenous molecular switch. In some embodiments, a polynucleotide encoding an at least partially reversible exogenous switch can be located in a genome, can be extrachromosomal or a combination thereof. In some embodiments, a stimulus can be an environmental stimulus. In some embodiments, a stimulus can be selected from a group consisting of a change in: pH, light, temperature, an electric current, microenvironment, a presence or absence of ions, a level of an ion, a mechanical stimulus, a presence of one or more ion type, a change in a culture medium composition and any combination thereof. In some embodiments, a stimulus can comprise a change in a temperature. In some embodiments, a temperature for at least partially anchorage independent proliferation can be from about 28° C. to about 34° C. In some embodiments, a temperature for at least partially anchorage dependent proliferation can be from about 37° C. to about 41° C. In some embodiments, a presence of a stimulus can cause anchorage dependent proliferation. In some embodiments, an absence of a stimulus can cause anchorage dependent proliferation. In some embodiments, a presence of a stimulus can cause anchorage independent proliferation. In some embodiments, an absence of a stimulus can cause anchorage independent proliferation. In some embodiments, a stimulus can be selected from a group consisting of a change in a presence, absence, or level of: an antibiotic, a protein, a chemical compound, a salt of any one of these and any combination thereof. In some embodiments, at least partially anchorage independent proliferation can be at least partially a result of an increase in expression of: integrin-linked kinase (ILK), cyclin D1, Cdk4, ST6 N-Acetylgalactosaminide Alpha-2, 6-Sialytransferase 5 (ST6GALNAC5), or a combination thereof. In some embodiments, an engineered cell can be grown in a bioreactor. In some embodiments, at least partially anchorage independent proliferation can be at least partially in a suspension. In some embodiments, anchorage dependent proliferation can be at least partially on, in, or around a scaffold. In some embodiments, a scaffold can be at least partially natural or synthetic. In some embodiments, a scaffold can comprise a silk, a polylactide, a polyglycolide, a polyester, a polycaprolactone, a chitosan, a hydrogel or a combination thereof. In some embodiments, an engineered cell can comprise any one selected from a group consisting of a c-MycER system, a Tet-on system, a Tet-off system and a combination thereof. In some embodiments, an engineered cell can comprise any one selected from a group consisting of a Cre-LoxP system, TALENS, Zinc Finger, a CRISPR system or a component thereof, and any combination thereof. In some embodiments, a polynucleotide encoding an at least partially reversible exogenous molecular switch can comprise DNA, RNA, or a combination thereof. In some embodiments, a polynucleotide encoding an at least partially reversible exogenous molecular switch can comprise DNA. In some embodiments, a polynucleotide encoding an at least partially reversible exogenous molecular switch can comprise cDNA. In some embodiments, a polynucleotide encoding an at least partially reversible exogenous molecular switch can comprise RNA. In some embodiments, a polynucleotide encoding an at least partially reversible exogenous molecular switch can comprise mRNA. In some embodiments, a polynucleotide encoding an at least partially reversible exogenous molecular switch can comprise an inducible promotor or operator, wherein a promoter or operator can be configured to repress or activate expression of a gene. In some embodiments, a promotor or operator can comprise any one selected from a group consisting of a tetracycline-controlled transcriptional unit, a dexamethasone-controlled transcriptional unit, a doxycycline-controlled transcriptional unit, a C-mycR transcriptional controlled unit and any combination thereof. In some embodiments, a polynucleotide encoding an at least partially reversible exogenous molecular switch can be codon optimized. In some embodiments, a polynucleotide encoding an at least partially reversible exogenous molecular switch can comprise an epigenetically modified base. In some embodiments, an epigenetically modified base can comprise a pyrimidine. In some embodiments, a pyrimidine can be a cytosine or a thymine. In some embodiments, an epigenetically modified base can comprise any one selected from a group consisting of a methylated base, a hydroxymethylated base, a formylated base, and a carboxylic acid containing base. In some embodiments, an epigenetically modified base can comprise a hydroxymethylated base. In some embodiments, a hydroxymethylated base can comprise a 5-hydroxymethylated base. In some embodiments, a 5-hydroxymethylated base can comprise a 5-hydroxymethylcytosine. In some embodiments, an epigenetically modified base can comprise a methylated base. In some embodiments, a methylated base can comprise a 5-methylated base. In some embodiments, a 5-methylated base can comprise a 5-methylcytosine. In some embodiments an isolated tissue can comprise an engineered cell described herein. In some embodiments, a tissue can comprise a plurality of polyester fibers. In some embodiments, at least a portion of a tissue can be tanned. In some embodiments, at least a portion of a tissue can be tanned with a tanning agent comprising: a chromium, an aluminum, a zirconium, a titanium, an iron, a sodium aluminum silicate, a formaldehyde, a glutaraldehyde, an oxazolidine, an isocyanate, a carbodiimide, a polycarbamoyl sulfate, a tetrakis, a hydroxyphosphonium sulfate, a sodium p-[(4,6-dichloro-1,3,5-triazin-2-yl)amino] benzenesulphonate, a pyrogallol, a catechol, a syntan, or any combination thereof. In some embodiments, at least a portion of a tissue can further comprise an extracellular matrix. In some embodiments, a leather can comprise at least a portion of an engineered cell, a derivative thereof, a progeny thereof or an isolated tissue. In some embodiments, a leather can be in a form of any one selected from a group consisting of a bag, a belt, a watch strap, a package, a shoe, a boot, a footwear, a glove, a clothing, a vest, a jacket, a pant, a hat, a shirt, an undergarment, a luggage, a clutch, a purse, a ball, a backpack, a wallet, a saddle, a harness, a chap, a whip, furniture, furniture accessories, an upholstery, an automobile car seat, an automobile interior, and any combination thereof. In some embodiments, a leather can comprise a biofabricated material. In some embodiments, a biofabricated material can comprise zonal properties. In some embodiments, a method can comprise contacting an engineered cell disclosed herein with a stimulus. In some embodiments, a stimulus can comprise any one selected from a group consisting of a change in: pH, light, temperature, an electric current, microenvironment, a presence or absence of ions, a change in mechanical stimulus, a level of an ion, a presence of one or more ion type, and any combination thereof. In some embodiments, a stimulus can comprise a change in a temperature. In some embodiments, engineered cells can be grown at a temperature for at least partially anchorage independent proliferation at from about 28° C. to about 34° C. In some embodiments, removal of a stimulus can abate at least partially anchorage independent proliferation. In some embodiments, engineered cells can be grown at a temperature for at least partially anchorage dependent proliferation at from about 37° C. to about 41° C. In some embodiments, removal of a stimulus can abate at least partially anchorage dependent proliferation. In some embodiments, an at least partially reversible exogenous molecular switch or a polynucleotide encoding an at least partially reversible exogenous molecular switch can be introduced into an engineered cell by transfection, electroporation, or transduction. In some embodiments, an at least partially reversible exogenous molecular switch or a polynucleotide encoding an at least partially reversible exogenous molecular switch can be introduced by any one selected from a group consisting of a vector, wherein a vector can be a virus, a viral-like particle, an adeno-associated viral vector, a liposome, a nanoparticle, a plasmid, linear dsDNA and a combination thereof. In some embodiments, a vector can comprise a plasmid. In some embodiments, proliferation of an engineered cell can be determined at least in part by a method selected from a group consisting of manual cell counting, automated cell counting and indirect cell counting. In some embodiments, proliferation of an engineered cell can be determined by use of a method selected from a group consisting of a counting chamber, colony forming unit counting, electrical resistance, flow cytometry, image analysis, stereologic cell counting, spectrophotometry, and impedance microbiology. In some embodiments, a method can comprise tanning an engineered cell disclosed herein. In some embodiments, tanning can comprise at least a portion of a tissue. In some embodiments, a tissue can comprise a layered structure. In some embodiments, a layered structure can comprise any one selected from a group consisting of a dermal layer, an epidermal layer, laminin, fibronectin, collagen and a combination thereof. In some embodiments, a tissue can comprise any one selected from a group consisting of a fibroblast, a keratinocyte, a corneocyte, a melanocyte, a Langerhans cell, a basal cell, an adipocyte, a smooth muscle cell, an epithelial cell, and a combination thereof. In some embodiments, a method can comprise selecting or screening for an engineered cell. In some embodiments a synthetic leather can comprise an engineered cell. In some embodiments, a synthetic leather, which prior to tanning can comprise a portion of a tissue. In some embodiments, a tissue can be at least partially subjected to further processing. In some embodiments, further processing can be selected from a group consisting of tanning, preserving, soaking, bating, pickling, depickling, thinning, retanning, lubricating, crusting, wetting, sammying, shaving, rechroming, neutralizing, dyeing, fatliquoring, filling, stripping, stuffing, whitening, fixating, setting, drying, conditioning, milling, staking, buffing, finishing, oiling, brushing, padding, impregnating, spraying, roller coating, curtain coating, polishing, plating, embossing, ironing, glazing, tumbling, and any combination thereof. In some embodiments, a synthetic leather can comprise a biofabricated material. In some embodiments, a biofabricated material can comprise zonal properties. In some embodiments, a culture vessel can comprise an engineered cell disclosed herein. In some embodiments, a culture vessel can comprise any one selected from a group consisting of a plastic, a metal, a glass and a combination thereof. In some embodiments, a culture vessel can comprise an agent that causes an engineered cell to adhere to at least a portion of a culture vessel. In some embodiments, an agent can comprise poly-L-lysine. In some embodiments, a manufacturing facility can comprise an engineered cell. In some embodiments, a kit can comprise an engineered cell disclosed herein. In some embodiments, a kit can further comprise a growth medium. In some embodiments, a kit can further comprise instructions for use.

Also disclosed herein are engineered cells. In some embodiments, an engineered cell can comprise an at least partially reversible exogenous molecular switch or a polynucleotide encoding an at least partially reversible exogenous molecular switch or a combination thereof. In some embodiments, an at least partially reversible exogenous molecular switch can cause an engineered cell to grow at least partially anchorage independent or at least partially anchorage dependent based on a direct or indirect stimulus. In some embodiments, proliferation of an engineered cell can be determined at least in part by a method selected from a group consisting of manual cell counting, automated cell counting and indirect cell counting. In some embodiments, an engineered cell can be a prokaryotic cell or eukaryotic cell. In some embodiments, an engineered cell can be an animal cell. In some embodiments, an engineered cell can be an isolated cell. In some embodiments, an engineered cell can be a non-human cell. In some embodiments, an engineered cell can be a human cell. In some embodiments, an engineered cell can be selected from a group consisting of a primate cell, bovine cell, ovine cell, porcine cell, equine cell, canine cell, feline cell, rodent cell, bird cell, and lagomorph animal cell. In some embodiments, an engineered cell can be a bovine cell. In some embodiments, an engineered cell can be an immortalized cell. In some embodiments, an engineered cell can be selected from a group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof. In some embodiments, an engineered cell can be a fibroblast, or a fibroblast-like cell. In some embodiments, an engineered cell can be derived from a group consisting of a pluripotent stem cell, a mesenchymal stem cell, induced pluripotent stem cell and an embryonic stem cell. In some embodiments, an engineered cell can be a cell from a cell line comprising a plurality of cells. In some embodiments, an engineered cell can express an exogenous oncogene or genes involved in regulation of cells proliferation. In some embodiments, an engineered cell can express a polypeptide or a biologically active fragment thereof encoded by: TERT, Bmi1, or any combination thereof. In some embodiments, an engineered cell can express a polypeptide or a biologically active fragment thereof encoded by: TERT, CcnD1, Cdk4, or any combination thereof. In some embodiments, an engineered cell can be modified to have enhanced extracellular matrix production as compared to an otherwise comparable wildtype cell that does not comprise an at least partially reversible exogenous molecular switch or a polynucleotide encoding an at least partially reversible exogenous molecular switch. In some embodiments, an extracellular matrix can comprise collagen type I, collagen type III, elastin, fibronectin, laminin, or a combination thereof. In some embodiments, an extracellular matrix can comprise collagen. In some embodiments, an at least partially reversible exogenous molecular switch or a polynucleotide encoding an at least partially reversible exogenous molecular switch can be configured to at least partially increase or decrease expression in response to a stimulus. In some embodiments, increased expression of an at least partially reversible exogenous molecular switch or a polynucleotide encoding an at least partially reversible exogenous molecular switch can cause at least partial anchorage dependent proliferation. In some embodiments, increased expression of an at least partially reversible exogenous molecular switch or a polynucleotide encoding an at least partially reversible exogenous molecular switch can cause at least partial anchorage independent proliferation. In some embodiments, decreased expression of an at least partially reversible exogenous molecular switch or a polynucleotide encoding an at least partially reversible exogenous molecular switch can cause at least partial anchorage dependent proliferation. In some embodiments, decreased expression of an at least partially reversible exogenous molecular switch or a polynucleotide encoding an at least partially reversible exogenous molecular switch can cause at least partial anchorage independent proliferation. In some embodiments, at least partial anchorage dependent proliferation can consume more, same or less nutrients or growth factors as compared to anchorage independent proliferation. In some embodiments, at least partially anchorage independent proliferation can consume more, same, or less nutrients or growth factors as compared to at least partially anchorage dependent proliferation. In some embodiments, an at least partially reversible exogenous molecular switch or a polynucleotide encoding an at least partially reversible exogenous molecular switch can be a single switch, or a plurality of switches. In some embodiments, a polynucleotide encoding an at least partially reversible exogenous molecular switch can encode a selectable marker. In some embodiments, a selectable marker can comprise a fluorescent protein. In some embodiments, an engineered cell can comprise a recombinant selectable marker. In some embodiments, a selectable marker can be selected from a group consisting of: an antibiotic resistance gene, a gene encoding a fluorescent protein, and a gene expressing an auxotrophic marker. In some embodiments, an engineered cell can comprise a polynucleotide encoding an at least partially reversible exogenous molecular switch. In some embodiments, a polynucleotide encoding an at least partially reversible exogenous molecular switch can be located in a genome, can be extrachromosomal or a combination thereof. In some embodiments, a stimulus can be an environmental stimulus. In some embodiments, a stimulus can be selected from a group consisting of a change in: pH, light, temperature, an electric current, microenvironment, a presence or absence of ions, a level of an ion, a mechanical stimulus, a presence of one or more ion type, a change in a culture medium composition and any combination thereof. In some embodiments, a stimulus can comprise a change in a temperature. In some embodiments, a temperature for at least partially anchorage independent proliferation can be from about 28° C. to about 34° C. In some embodiments, a temperature for at least partially anchorage dependent proliferation can be from about 37° C. to about 41° C. In some embodiments, a presence of a stimulus can cause anchorage dependent proliferation. In some embodiments, an absence of a stimulus can cause anchorage dependent proliferation. In some embodiments, a presence of a stimulus can cause anchorage independent proliferation. In some embodiments, an absence of a stimulus can cause anchorage independent proliferation. In some embodiments, a stimulus can be selected from a group consisting of a change in a presence, absence, or level of: an antibiotic, a protein, a chemical compound, a salt of any one of these and any combination thereof. In some embodiments, at least partially anchorage independent proliferation can be at least partially a result of an increase in expression of: integrin-linked kinase (ILK), cyclin D1, Cdk4, ST6 N-Acetylgalactosaminide Alpha-2, 6-Sialytransferase 5 (ST6GALNAC5), or a combination thereof. In some embodiments, an engineered cell can be grown in a bioreactor. In some embodiments, at least partially anchorage independent proliferation can be at least partially in a suspension. In some embodiments, anchorage dependent proliferation can be at least partially on, in, or around a scaffold. In some embodiments, a scaffold can be at least partially natural or synthetic. In some embodiments, a scaffold can comprise a silk, a polylactide, a polyglycolide, a polyester, a polycaprolactone, a chitosan, a hydrogel or a combination thereof. In some embodiments, an engineered cell can comprise any one selected from a group consisting of a c-MycER system, a Tet-on system, a Tet-off system and a combination thereof. In some embodiments, an engineered cell can comprise any one selected from a group consisting of a Cre-LoxP system, TALENS, Zinc Finger, a CRISPR system or a component thereof, and any combination thereof. In some embodiments, a polynucleotide encoding an at least partially reversible exogenous molecular switch can comprise DNA, RNA, or a combination thereof. In some embodiments, a polynucleotide encoding an at least partially reversible exogenous molecular switch can comprise DNA. In some embodiments, a polynucleotide encoding an at least partially reversible exogenous molecular switch can comprise cDNA. In some embodiments, a polynucleotide encoding an at least partially reversible exogenous molecular switch can comprise RNA. In some embodiments, a polynucleotide encoding an at least partially reversible exogenous molecular switch can comprise mRNA. In some embodiments, a polynucleotide encoding an at least partially reversible exogenous molecular switch can comprise an inducible promotor or operator, wherein a promoter or operator can be configured to repress or activate expression of a gene. In some embodiments, a promotor or operator can comprise any one selected from a group consisting of a tetracycline-controlled transcriptional unit, a dexamethasone-controlled transcriptional unit, a doxycycline-controlled transcriptional unit, a C-mycR transcriptional controlled unit and any combination thereof. In some embodiments, a polynucleotide encoding an at least partially reversible exogenous molecular switch can be codon optimized. In some embodiments, a polynucleotide encoding an at least partially reversible exogenous molecular switch can comprise an epigenetically modified base. In some embodiments, an epigenetically modified base can comprise a pyrimidine. In some embodiments, a pyrimidine can be a cytosine or a thymine. In some embodiments, an epigenetically modified base can comprise any one selected from a group consisting of a methylated base, a hydroxymethylated base, a formylated base, and a carboxylic acid containing base. In some embodiments, an epigenetically modified base can comprise a hydroxymethylated base. In some embodiments, a hydroxymethylated base can comprise a 5-hydroxymethylated base. In some embodiments, a 5-hydroxymethylated base can comprise a 5-hydroxymethylcytosine. In some embodiments, an epigenetically modified base can comprise a methylated base. In some embodiments, a methylated base can comprise a 5-methylated base. In some embodiments, a 5-methylated base can comprise a 5-methylcytosine. In some embodiments an isolated tissue can comprise an engineered cell disclosed herein. In some embodiments, a tissue can comprise a plurality of polyester fibers. In some embodiments, at least a portion of a tissue can be tanned. In some embodiments, at least a portion of a tissue can be tanned with a tanning agent comprising: a chromium, an aluminum, a zirconium, a titanium, an iron, a sodium aluminum silicate, a formaldehyde, a glutaraldehyde, an oxazolidine, an isocyanate, a carbodiimide, a polycarbamoyl sulfate, a tetrakis hydroxyphosphonium sulfate, a sodium p-[(4,6-dichloro-1,3,5-triazin-2-yl)amino] benzenesulphonate, a pyrogallol, a catechol, a syntan, or any combination thereof. In some embodiments, at least a portion of a tissue can further comprise an extracellular matrix. In some embodiments, a leather can comprise at least a portion of an engineered cell disclosed herein, a derivative thereof, a progeny thereof or an isolated tissue. In some embodiments, a leather can be in a form of any one selected from a group consisting of a bag, a belt, a watch strap, a package, a shoe, a boot, a footwear, a glove, a clothing, a vest, a jacket, a pant, a hat, a shirt, an undergarment, a luggage, a clutch, a purse, a ball, a backpack, a wallet, a saddle, a harness, a chap, a whip, furniture, furniture accessories, an upholstery, an automobile car seat, an automobile interior, and any combination thereof. In some embodiments, a leather can comprise a biofabricated material. In some embodiments, a biofabricated material can comprise zonal properties. In some embodiments, a method can comprise contacting an engineered cell disclosed herein with a stimulus. In some embodiments, a stimulus can comprise any one selected from a group consisting of a change in: pH, light, temperature, an electric current, microenvironment, a presence or absence of ions, a change in mechanical stimulus, a level of an ion, a presence of one or more ion type, and any combination thereof. In some embodiments, a stimulus can comprise a change in a temperature. In some embodiments, engineered cells can be grown at a temperature for at least partially anchorage independent proliferation at from about 28° C. to about 34° C. In some embodiments, removal of a stimulus can abate at least partially anchorage independent proliferation. In some embodiments, engineered cells can be grown at a temperature for at least partially anchorage dependent proliferation at from about 37° C. to about 41° C. In some embodiments, removal of a stimulus can abate at least partially anchorage dependent proliferation. In some embodiments, an at least partially reversible exogenous molecular switch or a polynucleotide encoding an at least partially reversible exogenous molecular switch can be introduced into an engineered cell by transfection, electroporation, or transduction. In some embodiments, an at least partially reversible exogenous molecular switch or a polynucleotide encoding an at least partially reversible exogenous molecular switch can be introduced by any one selected from a group consisting of a vector, wherein a vector can be a virus, a viral-like particle, an adeno-associated viral vector, a liposome, a nanoparticle, a plasmid, linear dsDNA and a combination thereof. In some embodiments, a vector can comprise a plasmid. In some embodiments, proliferation of an engineered cell can be determined at least in part by a method selected from a group consisting of manual cell counting, automated cell counting and indirect cell counting. In some embodiments, proliferation of an engineered cell can be determined by use of a method selected from a group consisting of a counting chamber, colony forming unit counting, electrical resistance, flow cytometry, image analysis, stereologic cell counting, spectrophotometry, and impedance microbiology. In some embodiments, a method can comprise tanning an engineered cell disclosed herein. In some embodiments, tanning can comprise at least a portion of a tissue. In some embodiments, a tissue can comprise a layered structure, or a structure comprising an engineered cell described herein. In some embodiments, a layered structure can comprise any one selected from a group consisting of a dermal layer, an epidermal layer, laminin, fibronectin, collagen and a combination thereof. In some embodiments, a tissue can comprise any one selected from a group consisting of a fibroblast, a keratinocyte, a corneocyte, a melanocyte, a Langerhans cell, a basal cell, an adipocyte, a smooth muscle cell, an epithelial cell and a combination thereof. In some embodiments, a method can comprise selecting or screening for an engineered cell disclosed herein. In some embodiments, a synthetic leather can comprise an engineered cell disclosed herein. In some embodiments, a synthetic leather, which prior to tanning can comprise a portion of a tissue. In some embodiments, a tissue can be at least partially subjected to further processing. In some embodiments, further processing can be selected from a group consisting of tanning, preserving, soaking, bating, pickling, depickling, thinning, retanning, lubricating, crusting, wetting, sammying, shaving, rechroming, neutralizing, dyeing, fatliquoring, filling, stripping, stuffing, whitening, fixating, setting, drying, conditioning, milling, staking, buffing, finishing, oiling, brushing, padding, impregnating, spraying, roller coating, curtain coating, polishing, plating, embossing, ironing, glazing, tumbling, and any combination thereof. In some embodiments, a synthetic leather can comprise a biofabricated material. In some embodiments, a biofabricated material can comprise zonal properties. In some embodiments, a culture vessel can comprise an engineered cell disclosed herein. In some embodiments, a culture vessel can comprise any one selected from a group consisting of a plastic, a metal, a glass and a combination thereof. In some embodiments, a culture vessel can comprise an agent that causes an engineered cell to adhere to at least a portion of a culture vessel. In some embodiments, an agent can comprise poly-L-lysine. In some embodiments, a manufacturing facility can comprise an engineered cell disclosed herein. In some embodiments, a kit can comprise an engineered cell disclosed herein. In some embodiments, a kit can further comprise a growth medium. In some embodiments, a kit can further comprise instructions for use.

### INCORPORATION BY REFERENCE

All publications, patents, and patent applications mentioned in this specification are herein incorporated by reference to a same extent as if each individual publication, patent, or patent application was specifically and individually indicated to be incorporated by reference.

### BRIEF DESCRIPTION OF THE DRAWINGS

FIG. 1A shows population doubling time in hours over the number of passages. Unmodified Bovine Dermal Fibroblasts (BDF) can be expanded in vitro for around 40 population doubling (PD) with an average population doubling time (PDT) of 35.5 hours. The primary BDF changes it's PDT over multiple passages with a minimal PDT of 21.6 hours and maximum PDT of 51.5 hours from passage 0 to passage 11. FIG. 1B shows in contrast, VL-001 (SV40-TAg transduced cells) cells can be expanded for at least 20 more passages after Puromycin selection to reach a cumulative PD of 100 at passage 25. This number is not indicative of senescence, rather this is when the expansion experiment was stopped. The average PDT of VL-001 cell line (21.2 hours) is 40% shorter than unmodified primary BDFs. VL-001 cell line also exhibited a more consistent PDT among serial passaging. VL-001 cell line has a minimum PDT of 19.1 hours and maximum PDT of 23.7 hours from passage 6 to passage 25 as shown in FIG. 1A. FIG. 1C and FIG. 1D show phase microscopy images of passage 9 Unmodified Bovine Dermal Fibroblasts (BDF), and passage 24 VL-001 (SV40-TAg transduced) cells. Senescent cells have not been observed in the extensively passaged VL-001 cell line, whereas senescence morphology can be identified in the later passaged unmodified primary BDFs.

FIG. 2A shows trichome blue stained cross sections of tissue from wild type bovine dermal fibroblasts. FIG. 2B shows trichome blue stained cross sections of tissue from VL-001 (SV40-TAg transduced) cells. A comparison of FIG. 2A and FIG. 2B shows VL-001 cells can form a tissue similar to wild-type dermal fibroblasts (BDF) on PET scaffold. Images were cross-sections of the tissue with Trichrome blue staining, which highlighted the collagen in tissues. Wild type bovine dermal fibroblasts and VL-001 (SV40-TAg transduced cells) were seeded on a PET scaffold at 500,000 cells/cm2. Wild type bovine dermal fibroblasts were cultured in a human platelet lysate (HPL)-based medium (DMEM+10% HPL+ascorbic acid-2-phosphate (AA2P)+TGFB1+ACD+Normocin) for 4 weeks. VL-001 were cultured in a fetal bovine serum (FBS)-based medium (DMEM+20% FBS+NEAA+Antibiotic/Antimycotic) for 2 weeks, before AA2P was added to the FBS-based medium for another 2 weeks.

FIG. 3 depicts a schematic diagram of the construct used for transection of cells with SV40-T antigen (SV40-Tag), with a puromycin reporter (Puro^{r}), and the construct used for green fluorescent protein (GFP) expression.

FIG. 4A shows -LV -Puro cells. FIG. 4B shows -LV+Puro cells. FIG. 4C shows SV40 TAg+Puro cells. FIG. 4D shows SV40 TAg+Puro cells.

FIG. 5 shows the amount of collagen in culture media (ug/ml) for BDF and VL-001 cells grown in tissue formation media and in tissue formation media+TGFb1+AA2P.

FIG. 6A shows tissue scanning images of BDF cells grown in HPL media. FIG. 6B shows 10× magnification tissue scanning images of BDF cells grown in HPL media 10×. FIG. 6C shows tissue scanning and images of VL-001 cells grown in HPL media. FIG. 6D shows 10× magnification tissue scanning images of VL-001 cells grown in HPL media. FIG. 6E shows tissue scanning images of VL-001 cells grown in 20% FBS media. FIG. 6F shows 10× magnification tissue scanning images of VL-001 cells grown in 20% FBS media.

FIG. 7 depicts the amount of collagen in culture media (ug/ml) for BDF and VL-001 cells grown in culture media over the number of weeks in culture.

FIG. 8 depicts the amount of collagen normalized by wet weight (ug/g) for BDF and VL-001 cells.

FIG. 9A shows VL-001 cell line at zero weeks of growth in culture. FIG. 9B shows VL-001 at one week of growth in culture. A clear aggregate formation was observed after one week of suspension culture.

FIG. 9C shows VL-001 at two weeks of growth in culture. FIG. 9D shows VL-001 at four weeks of growth in culture.

FIG. 10 shows the increase in expression of COL1 genes upon TGFB1 and AA2P treatment. The expression levels of COL1A1 and COL1A2 gene were up-regulated in both primary Bovine Dermal Fibroblasts (BDF) and the immortalized cell line (VL-001) upon exposure to Transforming Growth Factor beta 1 (TGFB1) and ascorbic acid-2-phosphate (AA2P).

FIG. 11A and FIG. 11B each show an artificial dermal layer grown from an immortalized bovine fibroblast line on a PLA scaffold.

FIG. 12 shows the tissues generated by VL-001 cells have similar total collagen protein levels to primary Bovine Dermal Fibroblast derived tissue. Measurements of the VL-001 S1 and VL-001 S2 were taken from biopsies of the tissues shown in FIG. 11A and FIG. 11B.

FIG. 13 shows a picrosirius red (PSR) stained section of a tissue biopsy from an immortalized bovine fibroblast line on a PLA scaffold. The tissue biopsies were fixed in a plastic resin and 5 um sections were generated and stained using picrosirius red (PSR). The staining shows that the VL-001 cells could deposit collagen protein throughout the PLA scaffold.

FIG. 14 shows images of VL-001 tissues at the crust phase (after drying to remove the excess water). VL-001 cells were seeded onto Bovine Calf Serum (BCS) coated Poly-L-Lactide Acid (PLA) three-dimensional non-woven scaffolds, grown for 4 weeks in a cell culture media consisting of 5% hPL (human platelet lysate), Heparin (2 mg/L), Non-essential amino acids (1× concentration), ascorbic acid (82 ug/L), Antibiotic-Antimycotic (1× concentration; 100 units/mL of Penicillin, 100 ug/mL of streptomycin, and 250 ng/mL of Gibco Amphotericin B).

### DETAILED DESCRIPTION OF THE DISCLOSURE

Several aspects are described below with reference to example applications for illustration. It should be understood that numerous specific details, relationships, and methods are set forth to provide a full understanding of the features described herein. Features described herein can be practiced without one or more of the specific details or with other methods. The features described herein are not limited by an illustrated ordering of acts or events, as some acts can occur in different orders and/or concurrently with other acts or events. Furthermore, not all illustrated acts or events may be required to implement a methodology in accordance with the features described herein.

The terminology used herein can be for the purpose of describing particular cases only and is not intended to be limiting. As used herein, the singular forms “a”, “an” and “the” are intended to include the plural forms as well, unless the context clearly indicates otherwise. Furthermore, to an extent that the terms “including”, “includes”, “having”, “has”, “with”, or variants thereof are used in either the detailed description and/or the claims, such terms are intended to be inclusive in a manner similar to the term “comprising”.

In this disclosure the term “about” or “approximately” can mean a range of up to 10% of a given value. In this disclosure the term “substantially” refers to something that can be done to a great extent or degree.

As used herein the term “fibroblast cell” can include a connective tissue cell that is found in the skin and tendons of a body. A fibroblast cell can be obtained from a biopsy of a skin or tendon. Fibroblast cells are also ubiquitous in many tissues and organs besides skin and tendons. A fibroblast cell can be a type of biological cell that synthesizes extracellular matrix and collagen. A fibroblast cell can be a type of biological cell that produces the structural framework (stroma) for animal tissues and plays a critical role in wound healing. Fibroblasts can be the most common cells of connective tissue in animals. A fibroblast cell can comprise a major part of a tissue layer grown in culture, which can be tanned into a leather.

As used herein the term “fibroblast-like cell” can refer to a fibroblast cell that has been grown in culture, has been immortalized, or a combination thereof. A fibroblast-like cell can comprise a cell that has been differentiated to have a morphology, phenotype, or combination thereof that substantially resembles a fibroblast cell. A fibroblast-like cell can comprise genetic or phenotypic alterations from a fibroblast cell, while still expressing substantially similar gene expression to a fibroblast cell. A fibroblast-like cell can have similar characteristics to a fibroblast cell such as the ability to synthesize collagen, extracellular matrix, the structural matrix (stroma) of animal tissue, or any combination thereof. A fibroblast-like cell can produce substantially similar levels of collagen compared to a fibroblast cell.

As used herein, the term “pluripotent stem cell” can refer to any precursor cell that has an ability to form any adult cell other than placenta.

As used herein, the term “embryonic stem cells” or “ES cells” or “ESC” can refer to precursor cells that have an ability to form any adult cell.

As used herein, the term “induced pluripotent stem cells” or “iPS cells” or “iPSCs” can refer to a type of pluripotent stem cell artificially derived from a non-pluripotent cell (e.g., an adult somatic cell). Induced pluripotent stem cells can be identical to embryonic stem cells in an ability to form any adult cell, but may not be derived from an embryo.

As used herein the term “isolated” can refer to a cell that has been removed from an animal or human body. An isolated cell can be grown in culture or in vitro. An isolated cell can be in contact with other isolated cells, a scaffold, a medium, or a combination thereof.

As used herein, the term “anchorage dependent” and “anchorage independent” proliferation can refer to cell proliferation in any medium, in any condition, in any apparatus, or any combination thereof. Anchorage independent growth can refer to cellular growth while not adhered to a substrate. Anchorage dependent growth can refer to cellular growth while at least partially adhered to a substrate (e.g., a scaffold). Anchorage independent growth may also be referred to as non-adherent growth, or growth in suspension. In some embodiments, anchorage independent and anchorage dependent growth can comprise a cell surrounded by a medium. In some embodiments, anchorage independent growth can comprise a cell not contacting another cell or surface (e.g., a scaffold). In some embodiments, in anchorage independent growth a surface can be another cell. In some embodiments, anchorage independent growth can be growth as single cells in culture. In some embodiments, anchorage dependent growth can be growth of cells contacting a plurality cells in culture (e.g. on a scaffold).

As used herein, the terms “decellularize” or “decellularized” can refer to the removal of cells from a cell layer. The term “at least partially decellularized” can refer to the removal of at least some cells from a cell layer. “Decellularization” can refer to the process of removing cells to make a decellularized cell layer, and can be achieved through methods such as salting, or the use of detergents.

As used herein, the term “synthetic leather” can be meant that the skin equivalents described herein can serve as a skin equivalent for any mammal or non-mammal. The disclosure can be practiced with human and non-human mammals, such as non-human primates and members of the bovine, ovine, porcine, equine, canine and feline species as well as rodents such as mice, rats and guinea pigs, members of the lagomorph family including rabbit, fish including shark and stingray, birds including ostrich and reptiles including lizards, snakes and crocodiles. In some embodiments, a synthetic leather can comprise an artificial leather. In some embodiments, a synthetic leather can comprise a cruelty-free leather, an eco-friendly leather, a plastic free leather, or any combination thereof. In some embodiments, a synthetic leather can comprise a tanned artificial cell layer, a tanned at least partially decellularized cell layer, or a combination thereof. In some embodiments, a cell layer or an at least partially decellularized cell layer can comprise a dermal layer, an epidermal layer, an at least partially decellularized dermal layer, an at least partially decellularized epidermal layer, or any combination thereof. In some embodiments, mammalian synthetic leather which can be formed can be dependent on a source of a cell used in an invention described herein, e.g., Keratinocytes and fibroblasts, e.g., when bovine keratinocytes and fibroblasts can be used to form a skin equivalent, a bovine synthetic leather can be formed.

Methods, compositions, kits and systems disclosed herein can be directed to engineered cells for producing synthetic leathers, artificial epidermal layers, artificial dermal layers, layered structures, products produced therefrom and methods of producing the same. In some embodiments, an engineered cell can be an animal cell, for example, a bovine cell. In some embodiments, an engineered cell can be immortalized. In some embodiments, an engineered cell can comprise a molecular switch, wherein a molecular switch can be a polynucleotide. In some embodiments, an engineered cell can be comprised in a synthetic leather. In some embodiments, a synthetic leather can comprise one or a plurality of layers. In some embodiments, one or a plurality of layers can comprise cells, wherein a cell can be cultured in vitro. In some embodiments, a method described herein can provide high-throughput methods that reliably, accurately, and reproducibly scale up to commercial levels a production of synthetic leather. Advantages of a synthetic leather, engineered epidermal equivalent, engineered full thickness skin equivalent and methods of making the same disclosed herein can include production of customized tissues in a reproducible, high throughput and easily scalable fashion with appealing appearance, texture, thickness, durability, or any combination thereof. In some embodiments, full thickness skin equivalent can comprise at least one dermal layer and at least one epidermal layer. In some embodiments, a full thickness skin equivalent and a full skin equivalent can be used interchangeably. In some embodiments, a dermal layer can comprise an engineered cell described herein.

In some embodiments, a synthetic leather disclosed herein can comprise a layer of artificial dermal layer comprising a fibroblast, an artificial epidermal layer comprising a keratinocyte, or a combination thereof. In some embodiments, a dermal layer and an epidermal layer can form a layered structure. In some embodiments, a synthetic leather can comprise one or more layered structures. In some embodiments, a synthetic leather can be tanned and further processed. In some embodiments, a cell forming a synthetic layer can comprise an immortalized bovine fibroblast. In some embodiments, a dermal layer can be placed on a scaffold, such as silk, to achieve natural leather thickness and texture. In some embodiments, a synthetic leather can comprise an artificial dermal layer comprising an engineered cell described herein.

In some embodiments, a method of making a synthetic leather can comprise forming a structure comprising an artificial dermal layer and tanning a structure. In some embodiments, a method can comprise further processing an artificial structure, e.g., to achieve natural leather thickness and texture.

In some embodiments, a synthetic leather can comprise a cell layer. In some embodiments, a synthetic leather can comprise multiple cell layers. In some embodiments, a synthetic leather can comprise an at least partially decellularized cell layer. In some embodiments, a cell layer can comprise a dermal layer, an epidermal layer, a tissue layer, a basement membrane, a basement membrane substitute, or any combination thereof. In some embodiments, a synthetic leather can further comprise hypodermis, scale, scute, osteoderm, or a combination thereof. In some embodiments, a synthetic layer can comprise a full thickness skin equivalent. In some embodiments, a full thickness skin equivalent can comprise any one or combination of layers disclosed herein. In some embodiments, a portion of one or more cell layers in a synthetic leather can be removed. In some embodiments, removing at least a portion of one or more cell layers can comprise at least partially decellularizing, shaving, or a combination thereof. In some embodiments, an at least partially decellularizing can comprise contacting a cell layer with a salt solution. In some embodiments a contacting with a salt solution can comprise immersing in a salt solution. In some embodiments, a salt solution can comprise sodium chloride, coarse salt crystals, brine solution, or a combination thereof. In some embodiments a brine solution can comprise about 1%, about 2%, about 3%, about 4%, about 5%, about 6%, about 7%, about 8%, about 9%, about 10%, about 11%, about 12%, about 12%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% salt. In some embodiments, a cell layer or an at least partially decellularized cell layer can be tanned. In some embodiments, a tanning can be performed after formation of one or more cell layers or layered structures. In some embodiments, a cell layers or layered structure, can comprise an engineered cell. In some embodiments, a tanning can be performed after an at least partial decellularization of a cell layer. In some embodiments, a synthetic leather can be further processed. In some embodiments, a cell layer can comprise a hair follicle cell, a melanocyte, or a combination thereof.

In some embodiments, a synthetic leather can comprise a dermal layer, or an at least partially decellularized portion thereof. In some embodiments, a dermal layer can be an engineered dermis equivalent, e.g., an artificial dermal layer formed in vitro.

In some embodiments, a dermal layer can comprise cells of a connective tissue. In some embodiments, a dermal layer can comprise a fibroblast. In some embodiments, a fibroblast in a dermal layer can express one or more markers including, but not limited to, a cluster of differentiation 10 (CD10), a cluster of differentiation 73 (CD73), a cluster of differentiation 44 (CD44), a cluster of differentiation 90 (CD90), a cluster of differentiation 105 (CD105), a type I collagen, a type III collagen, a prolyl-4-hydroxylase beta fibroblast, or a combination thereof. In some embodiments, a dermal layer can comprise other types of cells, such as immune cells, macrophages, adipocytes, or a combination thereof. In some embodiments, a dermal layer can comprise an engineered cell. In some embodiments, a cell layer can comprise an immortalized cell, a bovine cell, a fibroblast cell, or any combination thereof. In some embodiments, a cell layer can comprise an immortalized bovine fibroblast.

In some embodiments, a dermal layer can comprise a matrix component in addition to a cell. In some embodiments, a matrix component can include any one or more of collagen, elastin, an extrafibrillar matrix, an extracellular gel-like substance primarily composed of glycosaminoglycans, proteoglycans, glycoproteins, or any combination thereof. In some embodiments, an extracellular gel-like substance primarily composed of glycosaminoglycans can comprise a hyaluronan.

In some embodiments, a dermal layer can comprise a matrix support. In some embodiments, a matrix support can be a scaffold. In some embodiments, a matrix support can comprise a contracted collagen gel. In some embodiments, a pure collagen matrix can be a polyglygolic acid mesh or collagen and glycosaminoglycan matrix covered with a silastic membrane (C-GAG), a biopolymer, or any combination thereof. In some embodiments, a biopolymer can comprise chitosan. In some embodiments, a matrix can be seeded with fibroblasts. In some embodiments, seeding with a fibroblast can give rise to an organotypic model. In some embodiments, a cell layer can comprise a naturally derived dermis, a keratinocyte, or a combination thereof. In some embodiments, a naturally derived dermis can be obtained from an allogenic cadaver skin. In some embodiments, a keratinocyte can form a sheet of keratinocytes. In some embodiments, a cell layer can comprise a lyophilized devitalized dermis from cadaver skin to support a keratinocyte sheet.

In some embodiments, a thickness of leather units can be reported in millimeters, ounces, or irons. In some embodiments, one ounce can equal 1/64 in. or 0.0156 in. or 0.396 mm. In some embodiments, one iron can equal 1/48 in. or 0.0208 in. or 0.53 mm.

In some embodiments, a thickness of a dermal layer can be engineered to fit a function or use of a synthetic leather. In some embodiments, a dermal layer can have a thickness from about 0.01 mm to about 50 mm. In some embodiments, a dermal layer can have a thickness from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a dermal layer can have a thickness from about 0.02 mm to 5 mm. For example, a dermal layer can have a thickness from about 0.1 mm to 0.5 mm. In some embodiments, a dermal layer can have a thickness from about 0.2 mm to 0.5 mm. In some embodiments, a thickness of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a thickness of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a thickness of at least about 50 mm.

In some embodiments, a length of a dermal layer can be engineered to fit a function or use of a synthetic leather. In some embodiments, a dermal layer can have a length from about 0.01 mm to about 50 m. In some embodiments, a dermal layer can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a dermal layer can have a length from about 0.02 mm to 5 mm. For example, a dermal layer can have a length from about 0.1 mm to 0.5 mm. In some embodiments, a dermal layer can have a length from about 0.2 mm to 0.5 mm. In some embodiments, a length of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a length of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a length of at least about 50, 60, 70, 80, 90, 100, 200, 300, 400, 500, 700, 1000 mm. In some embodiments, a dermal layer can have a length of at least about 50, 60, 70, 80, 90, 100, 200, 300, 400, 500, 600, 700 cm. In some embodiments, a dermal layer can have a length of at least about 50, 60, 70, 80, 90, 100, 200, 300, 400 m.

In some embodiments, a width of a dermal layer can be engineered to fit a function or use of a synthetic leather. In some embodiments, a dermal layer can have a width from about 0.01 mm to about 50 m. In some embodiments, a dermal layer can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a dermal layer can have a width from about 0.02 mm to 5 mm. In some embodiments, a dermal layer can have a width from about 0.1 mm to 0.5 mm. In some embodiments, a dermal layer can have a width from about 0.2 mm to 0.5 mm. In some embodiments, a width of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a width of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a width of at least about 50, 60, 70, 80, 90, 100, 200, 300, 400, 500, 700, 1000 mm. In some embodiments, a dermal layer can have a width of at least about 50, 60, 70, 80, 90, 100, 200, 300, 400, 500, 600, 700 cm. In some embodiments, a dermal layer can have a width of at least about 50, 60, 70, 80, 90, 100, 200, 300, 400 m.

In some embodiments, a synthetic leather can comprise one or more dermal layers. In some embodiments, a synthetic leather can have at least 1, 2, 3, 4, 5, 6, 7, 8, 9, 10, 12, 14, 16, 18, 20, 40, 60, 80, or 100 dermal layers. In some embodiments, when a synthetic leather can comprise more than one dermal layer, a dermal layer can be placed upon another dermal layer. In some embodiments, a synthetic leather can comprise two dermal layers, e.g., a first dermal layer and a second dermal layer. In some embodiments, a first dermal layer can be placed upon a second dermal layer.

In some embodiments, a dermal layer or an at least partially decellularized portion thereof can be stratified, e.g., having a plurality of sublayers. In some embodiments, a sublayer can have different compositions, e.g., different concentrations of a fiber. In some embodiments, a sublayer of a dermal layer or an at least partially decellularized portion thereof can have a different thickness, density, or a combination thereof. In some embodiments, a dermal layer or an at least partially decellularized portion thereof can have a papillary dermal layer, a reticular dermal layer, an at least partially decellularized portion of any of these, or any combination thereof. In some embodiments, a papillary dermal layer or an at least partially decellularized portion thereof can comprise loose areolar connective tissue, loosely arranged fibers, an at least partially decellularized portion of these, or any combination thereof. In some embodiments, a loosely arranged fiber can comprise a collagen fiber. In some embodiments, a reticular dermal layer can comprise a dense irregular connective tissue, including collagen fibers and dermal elastic fibers.

In some embodiments, a dermal layer or an at least partially decellularized portion thereof can comprise a free collagen matrix or lattice, which can be contractile in all directions, and homogeneous. In some embodiments, fibroblasts (e.g., immortalized bovine fibroblasts), and where appropriate other types of cells of a dermis, can be distributed in a continuous collagen gel. In some embodiments, a dermis equivalent can comprise at least one matrix of collagen type I in which fibroblasts can be distributed. In some embodiments, a dermis equivalent can also contain other extracellular matrix constituents. In some embodiments, an extracellular matrix constituent can include collagens, e.g., collagen IV, laminins, entactin, fibronectin, proteoglycans, glycosaminoglycans or hyaluronic acid. In some embodiments, a dermal layer can contain collagen type IV and laminin, entactin, or a combination thereof. In some embodiments, a concentration of these various constituents can be adjusted. For example, In some embodiments, a concentration of laminin can be from about 1% to about 15% of a final volume. In some embodiments, a concentration of collagen IV can be from about 0.3% to about 4.5% of a final volume. In some embodiments, a concentration of entactin can be from about 0.05% to about 1% of a final volume. In some embodiments, a collagen can be a collagen of bovine origin, of rat origin, of fish origin, any other source of natural collagen or collagen produced by genetic engineering which allows contraction in a presence of fibroblasts, or any combination thereof. In some embodiments, a collagen can be from an unnatural source. In some embodiments, a matrix can be a gel of collagen which may not be taut, obtained by contraction both horizontally and vertically, which does not impose a preferential organization of fibroblasts. In some embodiments, a matrix, also termed “free”, may not adhere to a support and a volume thereof can be modified without limit, conferring on it a varying thickness and diameter. In some embodiments, a thickness of a dermis equivalent can be at least 0.05 cm and, In some embodiments, approximately from 0.05 to 2 cm. In some embodiments, a thickness can also be increased without harming an advantageous property of a skin equivalent or synthetic leather. In some embodiments, a thickness can be from about 3 mm to about 20 cm or more. In some embodiments, a synthetic leather can comprise only dermal layers.

In some embodiments, a synthetic leather can comprise an epidermal layer (e.g., an artificial epidermal layer). In some embodiments, an epidermal layer can be an engineered epidermis equivalent, e.g., an artificial epidermal layer formed in vitro.

In some embodiments, an epidermal layer can comprise one or more types of cells, including keratinocytes, melanocytes, Langerhans cells, Merkel cells, and inflammatory cells. In some embodiments, an epidermal layer can comprise keratinocytes. In some embodiments, keratinocytes in an epidermal layer can include epithelial keratinocytes, basal keratinocytes, proliferating basal keratinocytes, differentiated suprabasal keratinocytes, or any combination thereof.

In some embodiments, an epidermal layer can comprise an engineered cell. In some embodiments, an epidermal layer can comprise an immortalized cell.

In some embodiments, an epidermal layer can comprise at least basal keratinocytes, e.g., keratinocytes which may not be differentiated. In some embodiments, an epidermal layer can further comprise partially differentiated keratinocytes as well as fully differentiated keratinocytes. In some embodiments, one or more epidermal layers in a synthetic leather can be a transition from undifferentiated basal keratinocytes to fully differentiated keratinocytes as one progresses from a dermal-epidermal junction where a basal keratinocyte can be localized.

In some embodiments, basal keratinocytes can express hemidesmosomes, which can serve to help secure an epidermal and a dermal layer together. In some embodiments, basal keratinocytes can also serve to regenerate a skin. In some embodiments, an epidermal layer in a synthetic leather herein can have basal keratinocytes that serve these functions. In some embodiments, a synthetic leather comprising such basal keratinocytes can be capable of regeneration. In some embodiments, distinctions between basal keratinocytes and differentiated keratinocytes in one or more epidermal layers in a synthetic leather can be that both E- and P-cadherin's can be present in epidermal keratinocytes along a basal membrane zone (BMZ), but keratinocytes which may be differentiated and located away from a BMZ may only express E-cadherin.

In some embodiments, a basal keratinocyte of an epidermal layer can be aligned in a layer in direct contact with a dermal layer, serving as a boundary between a differentiated keratinocyte and a fibroblast. In alternative cases, there can be gaps between a basal keratinocytes and a dermal layer. Still further, there can be gaps between a basal keratinocyte and other basal keratinocytes, leaving gaps between a differentiated keratinocyte and a dermal layer. In these latter cases where there can be gaps between a basal or differentiated keratinocyte and a dermal layer, a dermal and epidermal layer may not be uniformly in contact with one another but can be adjacent to each other. In some embodiments, a dermal and an epidermal layer can be adjacent in that there can be generally fluid, but substantially no other intervening materials such as layers of cells, collagen, matrices or other supports between a dermal and an epidermal layer.

In some embodiments, keratinocytes in an epidermal layer can express one or more markers. In some embodiments, markers can include, but may not be limited to, Keratin 14 (KRT14), tumor protein p63 (p63), Desmoglein 3 (DSG3), Integrin, beta 4 (ITGB4), Laminin, alpha 5 (LAMA5), Keratin 5 (KRT5), an isoform of tumor protein p63 (e.g., TAp63), Laminin, beta 3 (LAMB3), and Keratin 18 (KRT18).

In some embodiments, a thickness of an epidermal layer can be engineered to fit a function or use of a synthetic leather. In some embodiments, an epidermal layer can have a thickness from about 0.001 mm to about 10 mm. In some embodiments, an epidermal layer can have a thickness from about 0.005 mm to about 10 mm, from about 0.005 mm to about 5 mm, from about 0.005 mm to about 2 mm, from about 0.01 mm to about 10 mm, from about 0.01 mm to about 5 mm, from about 0.01 mm to about 2 mm, from about 0.01 mm to about 1, from about 0.01 mm to about 0.8 mm, from about 0.01 mm to about 0.4 mm, from about 0.01 mm to about 0.2 mm, from about 0.01 mm to about 0.1 mm, from about 0.05 mm to about 0.4 mm, from about 0.05 mm to about 0.2 mm, from about 0.05 mm to about 0.1 mm, from about 0.1 mm to about 0.4 mm, from about 0.1 mm to about 0.2 mm, from about 0.08 mm to about 1 mm, or from about 0.05 mm to about 1.5 mm. In some embodiments, an epidermal layer can have a thickness from about 0.01 mm to about 2 mm. In some embodiments, an epidermal layer can have a thickness from about 0.1 mm to about 0.22 mm. In some embodiments, a thickness of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a thickness of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, thickness values described herein can be a thickness of an epidermal layer and a basement membrane substitute.

In some embodiments, a length of an epidermal layer can be engineered to fit a function or use of a synthetic leather. In some embodiments, an epidermal layer can have a length from about 0.01 mm to about 50 m. In some embodiments, an epidermal layer can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, an epidermal layer can have a length from about 0.02 mm to 5 mm. In some embodiments, an epidermal layer can have a length from about 0.1 mm to 0.5 mm. In some embodiments, an epidermal layer can have a length from about 0.2 mm to 0.5 mm. In some embodiments, a length of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a length of an epidermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, an epidermal layer can have a length of at least about 50, 60, 70, 80, 90, 100, 200, 300, 400, 500, 700, 1000 mm. In some embodiments, an epidermal layer can have a length of at least about 50, 60, 70, 80, 90, 100, 200, 300, 400, 500, 600, 700 cm. In some embodiments, an epidermal layer can have a length of at least about 50, 60, 70, 80, 90, 100, 200, 300, 400 m.

In some embodiments, a width of an epidermal layer can be engineered to fit a function or use of a synthetic leather. In some embodiments, an epidermal layer can have a width from about 0.01 mm to about 50 m. In some embodiments, an epidermal layer can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, an epidermal layer can have a width from about 0.02 mm to 5 mm. In some embodiments, an epidermal layer can have a width from about 0.1 mm to 0.5 mm. In some embodiments, an epidermal layer can have a width from about 0.2 mm to 0.5 mm. In some embodiments, a width of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a width of an epidermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, an epidermal layer can have a width of at least about 50, 60, 70, 80, 90, 100, 200, 300, 400, 500, 700, 1000 mm. In some embodiments, an epidermal layer can have a width of at least about 50, 60, 70, 80, 90, 100, 200, 300, 400, 500, 600, 700 cm. In some embodiments, an epidermal layer can have a width of at least about 50, 60, 70, 80, 90, 100, 200, 300, 400 m.

In some embodiments, a synthetic leather can comprise one or more epidermal layers. In some embodiments, a synthetic leather can have at least about 1, 2, 3, 4, 5, 6, 7, 8, 9, 10, 12, 14, 16, 18, 20, 40, 60, 80, or 100 epidermal layers. In some embodiments, when a synthetic leather can comprise more than one epidermal layer, one epidermal layer can be placed upon another epidermal layer. In some embodiments, a synthetic leather can comprise two epidermal layers, e.g., a first epidermal layer and a second epidermal layer. In some embodiments, a first epidermal layer can be placed upon a second epidermal layer.

In some embodiments, an epidermal layer can be stratified, e.g., having a plurality of sublayers. In some embodiments, sublayers can have different cell compositions, e.g., different types of keratinocytes. In some embodiments, sublayers can comprise engineered cells. In some embodiments, sublayers of an epidermal layer can have different thicknesses and/or densities. In some embodiments, an epidermal layer can have one or more of cornified layer (stratum corneum), clear/translucent layer (stratum lucidum), granular layer (stratum granulosum), spinous layer (stratum spinosum), basal/germinal layer (stratum basale/germinativum), or any combination thereof. In some embodiments, an epidermal layer can comprise functional epidermal permeability barrier (e.g., organized lipid bilayers in stratum corneum). In some embodiments, a stratum corneum, stratum lucidum, stratum granulosum, stratum spinosum, or stratum basale/germinativum, can have a thickness of about 0.0001 mm to about 5 mm. In some embodiments, a stratum corneum, stratum lucidum, stratum granulosum, stratum spinosum, or stratum basale/germinativum, can have a thickness of at least about 0.001 mm, 0.01 mm, 0.02 mm, 0.04 mm, 0.08 mm, 0.1 mm, 0.15 mm, 0.2 mm, 0.4 mm, 0.8 mm, 1 mm, 2 mm, 4 mm, 8 mm, or 10 mm. In some embodiments, a stratum corneum, stratum lucidum, stratum granulosum, stratum spinosum, or stratum basale/germinativum, can have a thickness of at most about 50 mm, 40 mm, 20 mm, 10 mm, 8 mm, 4 mm, 2 mm, 1 mm, 0.8 mm, 0.4 mm, 0.2 mm, 0.15 mm, 0.1 mm, 0.08 mm, 0.04 mm, 0.02 mm, or 0.01 mm.

In some embodiments, an epidermal layer can further comprise cells producing pigments, e.g., melanin. In some embodiments, such pigment-producing cells can be melanocytes. In some embodiments, melanocytes in a epidermal layer can express one or more markers. In some embodiments, such markers can include, but may not be limited to, SRY-box containing gene 10 (Sox-10), Microphthalmia-associated transcription factor (MITF-M), premelanosome protein (gp-100), Dopachrome tautomerase (DCT), Tyrosinase (TYR), and Melan-A (MLANA). In some embodiments, a synthetic leather may not comprise an epidermal layer.

In some embodiments, a synthetic leather can comprise collagen and extracellular matrix components produced by cells in a dermal layer and/or an epidermal layer disclosed herein. In some embodiments, a synthetic leather can comprise an at least partially decellularized dermal layer and/or an epidermal layer, as disclosed herein. In some embodiments, a synthetic leather does not comprise an epidermal layer. In some embodiments, a synthetic leather also can comprise at least a portion of hair follicle cells, endothelial cells, smooth muscle cells, dermal papilla cells, immune system cells (such as lymphocytes, dendritic cells, mast cells, macrophages or Langerhans cells), adipocytes, nerve cells, Schwann cells, and a mixture thereof. In some embodiments, a synthetic leather can comprise at least a portion of an engineered cell (e.g., a cell comprising a molecular switch). A synthetic leather can comprise an immortalized cell. In some embodiments, a synthetic leather can comprise an isolated cell. In some embodiments, a synthetic leather can comprise a cell line.

A synthetic leather can comprise at least a portion of prokaryotic cells, eukaryotic cells or a combination thereof. In some embodiments, a synthetic leather can comprise at least a portion of a bacterium cell, for example, Escherichia coli. In some embodiments, a synthetic leather can comprise at least a portion of a eukaryotic cell (e.g. a bovine cell, a porcine cell, a human cell, Saccharomyces cerevisiae).

In some embodiments, at least a portion of one or more cells in a synthetic leather can be genetically engineered cells. The term “genetically engineered” can refer to a man-made alteration to a nucleic acid content of a cell. Therefore, genetically engineered cells can include cells containing an insertion, deletion, and/or substitution of one or more nucleotides in a genome of a cell as well as alterations including an introduction of self-replicating extrachromosomal nucleic acids inserted into a cell. Genetically engineered cells also include those in which transcription of one or more genes has been altered, e.g., increased or reduced.

In some embodiments, a cell can comprise a molecular switch. In some embodiments, a cell may not have a molecular switch. In some embodiments, a cell can be a eukaryotic cell (e.g., an animal cell) or a prokaryotic cell. In some embodiments, a cell can be a genetically engineered cell. In some embodiments, a cell can be an isolated cell. In some embodiments, a cell can be an immortalized cell. In some embodiments, a cell can comprise a human cell, a fibroblast, a stem cell, or any combination thereof. A cell can be a fat tissue derived cell (e.g., adipocyte), a chondrocyte, an osteocyte, an osteoblast, a myofibroblast, a satellite cell, a myoblast, a myocyte, a keratinocyte, a corneocyte, a melanocyte, a Langerhans cell, a basal cell, a smooth muscle cell, an umbilical cord cell, a pluripotent stem cell, a mesenchymal stem cell, an embryonic stem cell, or any combination thereof. In some embodiments, a cell can be selected from a group consisting of a primate cell, a bovine cell, a ovine cell, a porcine cell, an equine cell, a canine cell, a feline cell, a rodent cell, a bird cell, a marsupial, a reptile, and a lagomorph animal cell. In some embodiments, a cell can be a genetically engineered cell. A cell can comprise a cell line with a plurality of cells. In some embodiments, a cell can produce extracellular matrix protein. In some embodiments, a cell can have enhanced extracellular matrix production as compared to an otherwise comparable wildtype cell that does not comprise a molecular switch. In some embodiments, extracellular matrix protein can comprise collagen, collagen type I, collagen type III, elastin, fibronectin, laminin or any combination thereof. In some embodiments, an engineered tissue can comprise a cell described herein.

In some embodiments, a cell can comprise one or more molecular switches. In some embodiments, a cell may not have a molecular switch. In some embodiments, activation or deactivation of a switch can generate a cascade of molecular actions within an engineered cell. In some embodiments, a cascade can comprise increased regulation of gene/protein expression. In some embodiments, a cascade can comprise decreased regulation of gene/protein expression. In some embodiments, a molecular switch can be a reversible molecular switch. In some embodiments, a molecular switch can be partially activated. In some embodiments, partial activation can occur from low levels or concentrations of a stimulus, such as an antibiotic that drives expression of a gene. For example, partial activation can comprise decreased expression of a switch as compared to full activation (e.g. a Tet-on system). In another example, partial activation can comprise increased expression of a switch as compared to full activation (e.g. a Tet-off system). In some embodiments, a molecular switch can be active in a portion of cells. In some embodiments, portion of cells can comprise more than about 5%, 10%, 20%, 30%, 40%, 50%, 60%, 70%, 80%, 90%, 95%, or 99% of cells being activated. In some embodiments, a portion of cells can comprise less than about 5%, 10%, 20%, 30%, 40%, 50%, 60%, 70%, 80%, 90%, 95%, or 99% of cells being activated. In some embodiments, a portion of cells can comprise about: 5% to about 99%, 10% to about 30%, 20% to about 50%, 40% to about 70%, or about 50% to about 95%, of cells being activated. In some embodiments, a switch can be a gene, a plurality of genes, a polynucleotide, or any combination thereof. In some embodiments, a polynucleotide can be DNA, RNA or a combination thereof. In some embodiments, a molecular switch can comprise DNA, cDNA, RNA, siRNA, mRNA, miRNA or any combination thereof. A molecular switch can increase or decrease gene expression after direct or indirect interaction with a stimulus. A molecular switch can be codon optimized. In some embodiments, a molecular switch can integrate into the genome, be extrachromosomal, or a combination of both. In some embodiments, a gene can be integrin-linked kinase (ILK), cyclin D1, CDK4, a gene in a p53-mediated apoptosis pathway or any combination thereof. A gene can be from a human, a mammal, a bird, a reptile, an amphibian, a fish, an invertebrate, a virus, a bacterium, or any combination thereof. In some embodiments, a gene can be fusion gene that can comprise of one or more genes. In some embodiments, a gene can encode for a temperature sensitive switch, for example, tsA58. In some embodiments, a gene can encode for a Cre-LoxP recombinase system a CRISPR system, a FLP-FRT system, a transcription activator-like effector nuclease (TALEN), a zinc finger, a methylation system, or any combination thereof. In some embodiments, a switch can be a tetracycline transcriptional unit, a dexamethasone-controlled transcriptional unit, a doxycycline-controlled transcriptional unit, a C-mycR controlled transcriptional unit, an antibiotic controlled transcriptional unit, a metabolite controlled transcriptional unit, or any combination thereof. In some embodiments, a molecular switch can comprise a Tet-on, a Tet-off system or any combination thereof. In some embodiments, a molecular switch can comprise a T-REx system. In some embodiments, in a Tet-on system, a system can be induced to express a gene when tetracycline, a derivative thereof, a salt thereof or any combination thereof may be introduced into a cell's environment. In some embodiments, in a Tet-off system, a system can be induced to repress a gene when a tetracycline, a derivative thereof, a salt thereof or any combination thereof may be introduced into a cell's environment. In some embodiments, a molecular switch can be an estrogen receptor (ER) system. In some embodiments, a molecular switch can comprise a mixture of inducible systems, for example an estrogen receptor system and a Tet-off system. In some embodiments, a switch can be a promotor, an operator, or a combination thereof. In certain cases, a promotor or an operator can change an expression of a gene. In some embodiments, a promotor, an operator or any combination thereof can be configured to activate or repress expression in a gene. In some embodiments, a switch can comprise a gene, a promotor, a polynucleotide sequence, or any combination thereof. In some embodiments, a molecular switch can at least partially cause anchorage independent or at least partially cause anchorage dependent proliferation based on a stimulus. In some embodiments, changes in expression of a molecular switch (e.g., increased or decreased expression) can at least partially cause anchorage independent or at least partially cause anchorage dependent proliferation. In some embodiments, a molecular switch can be at least partially on during proliferation. In some embodiments, a molecular switch can be at least partially off during proliferation.

In some embodiments, a cell can comprise a selectable marker (e.g., a reporter construct). In some embodiments, a switch can comprise a selectable marker. In some embodiments, a selectable marker can comprise green fluorescent protein (GFP), red fluorescent protein (RFP), yellow fluorescent protein (YFP), EGFP, a blue fluorescent protein, a cyan fluorescent protein, orange fluorescent protein, a gene encoding for any of these, or any combination thereof. In some embodiments, a selectable marker can comprise an antibiotic resistance gene (e.g., blasticidin, geneticin, mycophenolic acid, puromycin, zeocin, hygromycin b, a salt thereof or any combination thereof), a fluorophore, a biosynthesis gene, an auxotrophic marker, or any combination thereof. In some embodiments, a selectable marker can be antisense RNA. In some embodiments, a selectable marker can be used to select or screen for an engineered cell.

In some embodiments, a switch can comprise an epigenetic modification. In some embodiments, an epigenetic modification can be passed to daughter cells. In some embodiments, an epigenetic modification can be maintained for a single generation. In some embodiments, an epigenetic modification can be a nucleotide base, a sugar, or any combination thereof. In some embodiments, an epigenetic modification can comprise a pyrimidine, a purine or any combination thereof. In some embodiments, an epigenetic modification can comprise a ribose, a deoxyribose or any combination thereof. In some embodiments, an epigenetic modification can be on a cytosine, an adenine, a guanine, a threonine, an uracil or any combination thereof. An epigenetically modified base can comprise a methylated base, a hydroxymethylated base, a formylated base, a carboxylic acid containing base or any combination thereof. In some embodiments, an epigenetically modified base can be a 5-methylated base, 5-hydroxymethylated base, a 5-formylated base, 5-carboxylated base, a 5-hydroxymethycytosine, or any combination thereof.

In some embodiments, a molecular switch can be introduced into a cell by transfection, transformation, transduction or injection. In some embodiments, transfection or transformation can include electroporation, bolistic particle delivery, sonoporation or a combination thereof. In some embodiments, a polynucleotide encoding a molecular switch can be located in a genome or extrachromosomal. In some embodiments, a molecular switch can be delivered to a cell by a vector. In some embodiments, a vector can be a polynucleotide in a form of a plasmid. In some embodiments, a vector can be comprised of a liposome, a nanoparticle or any combination thereof. A liposome can include but may not be limited to unilamellar liposome, multilamellar liposome, archaeosome, noisome, novasome, cryptosome, emulsome, vesosome, or a derivative of any of these, or any combination thereof. A nanoparticle can include but may not be limited to a biopolymeric nanoparticle, alginate nanoparticle, xanthan gum nanoparticle, cellulose nanoparticle, dendrimer, polymeric micelle, polyplex, inorganic nanoparticle, nanocrystal, metallic nanoparticle, quantum dot, protein nanoparticle, polysaccharide nanoparticle, or a derivative of any of these, or any combination thereof. In some embodiments, a vector can be an RNA viral vector which can include but may not be limited to a retrovirus, lentivirus, coronavirus, alphavirus, flavivirus, rhabdovirus, morbillivirus, picornavirus, coxsackievirus, or picomavirus or portions of any of these, or fragments of any of these, or any combination thereof. In some embodiments, a vector can be a DNA viral vector which can include but may not be limited to an adeno-associated viral (AAV) vector, adenovirus, hybrid adenoviral system, hepadnavirus, parvovirus, papillomavirus, polyomavirus, herpesvirus, poxvirus, a portion of any of these, a fragment of any of these, or any combination thereof.

In some embodiments, a molecular switch can be controlled by a stimulus. In some embodiments, a stimulus can be a direct stimulus, an indirect stimulus or any combination thereof. In some embodiments, a stimulus can be a gradient. A gradient stimulus can partially cause a molecular switch to activate in a mixture of cells. In some embodiments, a gradient stimulus cannot active a molecular switch in a fraction of cells of a cell culture. In some embodiments, a gradient stimulus can active a molecular switch in a fraction of cells of a cell culture. In some embodiments as a gradient increases or decreases, a molecular switch can become more or less activated. In some embodiments, cell proliferation, cell seeding, or any combination thereof can be in a presence of a stimulus. In some embodiments, cell proliferation, cell seeding, or any combination thereof can be in an absence of a stimulus. In some embodiments, a cell (e.g., an immortalized cell with a switch) can come into contact with a stimulus. In some embodiments, a stimulus can cause a cell to proliferate at least partially anchorage independently. In some embodiments, a stimulus can cause a cell to proliferate at least partially anchorage dependently. In some embodiments, an absence of a stimulus can cause a cell to proliferate at least partially anchorage independently. In some embodiments, an absence of a stimulus can cause a cell to proliferate at least partially anchorage dependently. A cell can be exposed to one stimulus or a plurality of stimuli (e.g., a second stimulus). A stimulus can be an environmental stimulus. In some embodiments, an environmental stimulus can comprise a presence, absence or level of a temperature, a magnetic field, a pH, a light, an electric current, a microenvironment, an ion, a sound, air pressure, a humidity, a physical stimulus (e.g., a mechanical stimulus), or any combination thereof. In some embodiments, a stimulus can be a change in a medium. In some embodiments, a medium can comprise a culture medium. In some embodiments, a culture medium can comprise Dulbecco's modified eagle's medium (DMEM), RPMI-1640, Eagle's Minimal Essential Medium, Ham's nutrient mixtures, Iscove's modified, Dulbecco's medium, or any combination thereof. In some embodiments, a supplement can be added to a culture medium. In some embodiments, a supplement can comprise a salt, a buffering reagent, a phenol red, a 4-(2-hydroxyethyl)-1-piperazineethanesulfonic acid (HEPES) buffer, an amino acid, a carbohydrate, a lipid, a protein, a peptide, a fatty acid, a vitamin, an element, an antibiotic, a serum, a cell culture medium supplement, or any combination thereof. In some embodiments, a stimulus can be a presence, an absence, or a level of a carbohydrate, a lipid, a nucleic acid, a protein, an antibiotic, an organic chemical, an inorganic chemical, an artificial chemical, a metabolite, or any combination thereof. In some embodiments, a cell (for example an engineered cell described herein) can be exposed to a temperature of about: 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C., during a proliferation. In some embodiments, a cell (for example an engineered cell described herein) can be exposed to a temperature of about: 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C., during a seeding. In some embodiments, a temperature can be a temperature difference between proliferation and seeding can be about: 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C. or 35° C. In some cases a temperature stimulus can be a temperature change from about: 28° C. to about 36° C., 28° C. to about 37° C., 28° C. to about 38° C., 28° C. to about 39° C., 28° C. to about 40° C., 28° C. to about 41° C. 29° C. to about 36° C., 29° C. to about, 37° C., 29° C. to about 38° C., 29° C. to about 39° C., 29° C. to about 40° C., 29° C. to about 41° C., 30° C. to about 36° C., 30° C. to about 37° C., 30° C. to about 38° C., 30° C. to about 39° C., 30° C. to about 40° C., 30° C. to about 41° C., 31° C. to about 36° C., 31° C. to about 37° C., 31° C. to about 38° C., 31° C. to about 39° C., 31° C. to about 40° C., 31° C. to about 41° C., 32° C. to about 36° C., 32° C. to about 37° C., 32° C. to about 38° C., 32° C. to about 39° C., 32° C. to about 40° C., 32° C. to about 41° C., 33° C. to about 36° C., 33° C. to about 37° C., 33° C. to about 38° C., 33° C. to about 39° C., 33° C. to about 40° C., 33° C. to about 41° C., 34° C. to about 36° C., 34° C. to about 37° C., 34° C. to about 38° C., 34° C. to about 39° C., 34° C. to about 40° C., 34° C. to about 41° C. 35° C. to about 36° C., 35° C. to about 37° C., 35° C. to about 38° C., 35° C. to about 39° C., 35° C. to about 40° C., or 35° C. to about 41° C. In some cases a temperature stimulus can be a temperature change from about: 41° C. to about 28° C., 41° C. to about 29° C., 41° C. to about 30° C., 41° C. to about 31° C., 41° C. to about 32° C., 41° C. to about 33° C., 41° C. to about 34° C., 41° C. to about 35° C., 40° C. to about 28° C., 40° C. to about 29° C., 40° C. to about 30° C., 40° C. to about 31° C., 40° C. to about 32° C., 40° C. to about 33° C., 40° C. to about 34° C., 40° C. to about 35° C., 39° C. to about 28° C., 39° C. to about 29° C., 39° C. to about 30° C., 39° C. to about 31° C., 39° C. to about 32° C., 39° C. to about 33° C., 39° C. to about 34° C., 39° C. to about 35° C., 38° C. to about 28° C., 38° C. to about 29° C., 38° C. to about 30° C., 38° C. to about 31° C., 38° C. to about 32° C., 38° C. to about 33° C., 38° C. to about 34° C., 38° C. to about 35° C., 37° C. to about 28° C., 37° C. to about 29° C., 37° C. to about 30° C., 37° C. to about 31° C., 37° C. to about 32° C., 37° C. to about 33° C., 37° C. to about 34° C., 37° C. to about 35° C., 36° C. to about 28° C., 36° C. to about 29° C., 36° C. to about 30° C., 36° C. to about 31° C., 36° C. to about 32° C., 36° C. to about 33° C., 36° C. to about 34° C., or 36° C. to about 35° C. In some embodiments, a molecular switch can be reversible based on a temperature stimulus.

In some embodiments, a genetically engineered cell can comprise a gene for collagen production. In some embodiments, a collagen gene can be P4HA, P4HB, COL1A1, COL1A2, COL2A1, COL3A1 or any combination thereof. In some embodiments, a collagen gene can have an altered promotor that can change an expression of a collagen gene, e.g., increase or decrease. In some embodiments, a collagen gene can be from a human. In some embodiments, a collagen gene can be from an animal, a mammal, a bird, a reptile, an amphibian, a fish, an invertebrate, or any combination thereof.

In some embodiments, a synthetic leather can comprise an immortalized cell, a tissue developed therefrom or any combination thereof. In some embodiments, an exogenous polynucleotide can encode: (i) a polypeptide which interacts with a tumor suppressor protein or fragment thereof and can alter an activity of a tumor suppressor protein or fragment thereof, (ii) a polynucleotide that can encode a polypeptide which interacts with a tumor suppressor protein or fragment thereof, or (iii) a combination of (i) and (ii). In some embodiments, an activity of a tumor suppressor protein or fragment thereof can be measured by an in vitro assay. In some embodiments, an immortalized cell can comprise a molecular switch. In some embodiments, a synthetic leather may be from an non-immortalized cell, a tissue developed therefrom or any combination thereof. An immortalized cell can comprise a fibroblast cell, a fat tissue derived cell, an umbilical cord cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, or any combination thereof. In some embodiments, an immortalized cell can comprise or be derived from a pluripotent stem cell, an induced pluripotent stem cell, a mesenchymal stem cell, or an embryonic stem cell. In some embodiments, an immortalized cell can have similar characteristics to any cell type described herein. In some embodiments, an immortalized cell can have a mutation. In some embodiments, an immortalized cell can comprise an exogenous gene. In some embodiments, an exogenous gene or a plurality of exogenous genes can cause immortalization. In some embodiments, an exogenous gene can comprise hTERT, TERT, Bmi1, CcnD1, a mutant of Cdk4, Cdk4, TAg (SV40 large T), SV40, c-myc, H-ras, Ela, c-mMycER^{TAM}, E6, E7, HER-2, SRC, EGFR, Abl, Atk02, Aml1, Axl, Bcl, Dbl, EGFR, ERBB, Ets-1, Fins, Fos, Fps, Gli, Gsp, Her2, Hox11, Hst, 1-3, Int-2, Jun, Kit, KS3, K-SAM, Lbc, Lck, L-myc, Lyl-1, Lyt-10, Mas, MDM-2, Mll, Mos, Myb, Neu, N-Myc, Ost, Pax-5, Pim-1, PRAD-1, Ras-K, Ras-N, Ret, Ros, Ski, Sis, Set, Src, Tall, Tan1, Tiam1, Tsc2, Trk, or any combination thereof. In some embodiments, an immortalized cell can have an exogenous gene, which can be a fusion gene that can comprise one or more genes. In some embodiments, an immortalized cell can have a protein product or a biologically active fragment thereof that can induce immortalization. In some embodiments, an immortalized cell can have an exogenous gene removed after cell divisions (e.g., by a Cre-LoxP system or a CRISPR system). In some embodiments, an exogenous gene can be from a human. In some embodiments, an exogenous gene can be from a mammal, a bird, a reptile, an amphibian, a fish, an invertebrate, a virus, a bacterium, or any combination thereof. In some embodiments, an immortalized cell can have a random mutation or a plurality of mutations. In some embodiments, a mutation can be generated by UV mutagenesis, chemical mutagenesis, or any combination thereof. In certain cases, an immortalized cell can have a targeted mutation, for example, a targeted mutation may be made by a CRISPR system. In some embodiments, a mutation can be in a cell cycle gene, an oncogene, a metabolic gene, or any combination thereof. In some embodiments, an immortalized cell can have a mutation in a gene, a promotor region, an intragenic region, an intergenic region, or any combination thereof. In some embodiments, a gene can comprise an oncogene, a cell cycle gene, or a combination thereof. In some embodiments, an immortalized cell can have increased or decreased expression of an oncogene or genes involved in a regulation of cell proliferation. In some embodiments, an immortalized cell can have a molecular switch. In some embodiments, an immortalized cell can be a conditional immortalized cell. In some embodiments, a conditional immortalized cell can display an immortalized cellular phenotype under a certain environmental stimulus or a differentiated cellular phenotype under a different environmental stimulus. In some embodiments, an immortalized cell can be grown past about 30 cell divisions, about 40 cell divisions, about 50 cell divisions, about 60 cell divisions, about 70 cell divisions, about 80 cell divisions, about 90 cell divisions, about 100 cell divisions, about 150 cell divisions, about 200 cell divisions, about 250 cell divisions, about 300 cell divisions, about 350 cell divisions, about 400 cell divisions, about 450 cell divisions, about 500 cell divisions, about 550 cell divisions, about 600 cell divisions, about 650 cell divisions, about 700 cell divisions, about 750 cell divisions, about 800 cell divisions, about 850 cell divisions, about 900 cell divisions, about 950 cell divisions, about 1000 cell divisions, about 5,000 cell divisions, about 10,000 cell divisions, about 50,000 divisions, or about 100,000 cell divisions.

In some embodiments, a method described herein can comprise proliferating an immortalized cell in a first environment, wherein an immortalized cell can comprise a reversible exogenous molecular switch that can be at least partially activated or partially silenced by a presence or absence of a stimulus; and seeding an immortalized cell onto a scaffold. In some embodiments, an environment can comprise a bioreactor, an incubator, a vessel, a scaffold, a growth condition (e.g., growth medium, temperature, aeration, in suspension) or any combination thereof.

In some embodiments, a cell described herein can be comprised in a kit. In some embodiments, a kit can comprise a growth medium, instructions for use, packaging or any combination thereof.

In some embodiments, a synthetic leather can have at least one component of native skin such as melanocytes, hair follicles, sweat glands and nerve endings. In certain cases, a synthetic leather can be distinguished from normal native skin by its lack of at least one of these components. In some embodiments, displaying abnormal phenotypes or having at least one cell with an altered genotype, a synthetic leather can include all of these components.

In some embodiments, additional components can be added to a synthetic leather. Such additional components can include myoepithelial cells, duct cells, secretory cells, alveolar cells, Langerhans cells, Merkel cells, adhesions, mammary glands, or any mixture thereof. In some embodiments, a synthetic leather can comprise one or more of: neural cells, connective tissue (including bone, cartilage, cells differentiating into bone forming cells and chondrocytes, and lymph tissues), epithelial cells (including endothelial cells that form linings in cavities and vessels or channels, exocrine secretory epithelial cells, epithelial absorptive cells, keratinizing epithelial cells, and extracellular matrix secretion cells), and undifferentiated cells (such as embryonic cells, stem cells, and other precursor cells).

In some embodiments, a synthetic leather can comprise hair follicles. A hair follicle can comprise one or more structures, including papilla, matrix, root sheath, bulge, infundibulum, an arrector pili muscle, a sebaceous gland, an apocrine sweat gland, or any combination thereof. A hair follicle can comprise one or more hair follicle cells, including dermal papilla cell, outer root sheath cell, or any combination thereof. In some embodiments, a hair follicle can be in an epidermal layer. In some embodiments, a hair follicle can be in a dermal layer. In some embodiments, a hair follicle cell can be differentiated from a progenitor, e.g., a stem cell. In some embodiments, at least about 1%, 2%, 3%, 4%, 5%, 6%, 7%, 8%, 9%, 10%, 20%, 30%, 40%, 50%, 60%, 70%, 80%, 90%, 100% of hair follicle cells can be differentiated from an induced pluripotent stem cell.

In some embodiments, a synthetic leather can be devoid of hair, blood vessels, sebaceous glands, hair follicle, oil glands, nerve, or any combination thereof

In some embodiments, a synthetic leather can comprise a hair. In some embodiments, a synthetic leather can comprise a hair in one or more layered structures. In some embodiments, a synthetic leather can comprise a fur. In some embodiments, a hair (e.g., fur) can be natural, synthetic, or a combination thereof. In some embodiments, a hair (e.g., fur) can be grown from cells in a synthetic leather or added to a synthetic leather from an exogenous source. In some embodiments, a synthetic leather may not have any hair.

In some embodiments, at least a portion of one or more cells in a synthetic leather can be differentiated from a progenitor cell, such as a stem cell. In some embodiments, a synthetic leather can be generated from an engineered cell, or a tissue comprising an engineered cell as disclosed herein. In some embodiments, a fibroblast in a synthetic leather can be differentiated from a stem cell. In some embodiments, a keratinocyte in a synthetic leather can be differentiated from a stem cell. In some embodiments, a melanocyte in a synthetic leather can be differentiated from a stem cell.

In some embodiments, a stem cells can comprise an embryonic stem cell (ESC), an adult stem cell, a somatic stem cell, a tissue-specific stem cell, a mesenchymal stem cell, an induced pluripotent stem cell (iPSC), or any combination thereof. In some embodiments, a stem cell can be totipotent, pluripotent or multipotent. In some embodiments, a stem cell can comprise an adult stem cell, a cord blood stem cell, or a combination thereof. Embryonic stem cells can be derived from fertilized embryos that may be less than one week old. Induced pluripotent stem cells can be obtained through an induced expression of one or more of Oct3, Oct4, Sox2, Klf4, TERT, Bmi1, CcnD1, Cdk4, SV40 large T antigen, c-Myc, a fragment of any of these genes, in any somatic cell. In some embodiments a somatic cell can comprise an adult somatic cell. In some embodiments, a somatic cell can comprise a fibroblast. In some embodiments, an exogenous vector can comprise or encode a gene to induce pluripotency. In some embodiments, an exogenous vector can comprise a plasmid. In some embodiments, an induced pluripotent stem cell can be obtained by an active protein product or a biologically active fragment thereof. In some embodiments, one or more other genes can also be induced for reprograming a somatic cell to an induced pluripotent stem cell. In some embodiments a gene for inducing pluripotency can comprise NANOG, UTF1, LIN28, SALL4, NR5A2, TBX3, ESSRB, DPPA4, SV40LT, REM2, MDM2, and cyclin D1. In some embodiments, a gene can be from a human. In some embodiments, a gene can be from a mammal, a bird, a reptile, an amphibian, a fish, an invertebrate, or any combination thereof.

In some embodiments, various delivery methods can be used to modulate an expression of a gene to reprogram a somatic cell to an iPSC. In some embodiments, an exemplary delivery method can include a naked DNA delivery, an adenovirus vector, an electrical delivery, a chemical delivery, a mechanical delivery, a polymer-based system, a microinjection, a retrovirus vector (e.g., MMLV-derived retroviruses), a lentivirus vector (e.g., excisable lentiviruses), or any combination thereof. In some embodiments, a somatic cell can comprise an adult somatic cell. In some embodiments, a somatic cell can be transfected with a vector for delivery of a gene inducing pluripotency. In some embodiments, a vector can comprise a viral vector. In some embodiments, a vector can comprise a retroviral vector. In some embodiments, a gene inducing pluripotency can comprise Oct3, Oct4, Sox2, Klf4, TERT, Bmi1, CcnD1, Cdk4, SV40 large T antigen, c-Myc, a fragment of any of these, or any combination thereof. In some embodiments, a Sendai virus can be used as a delivery system. In some embodiments, a somatic cell can comprise an adult somatic cell. In some embodiments, a somatic cell can be transfected with an extrachromosomal vector. In some embodiments, an extrachromosomal vector can comprise a plasmid. In some embodiments, an extrachromosomal vector can deliver Oct3, Oct4, Sox2, Klf4, TERT, Bmi1, CcnD1, Cdk4, SV40 large T antigen, c-Myc, a fragment of any of these, or any combination thereof.

Disclosed herein in some embodiments, are methods and compositions comprising a cell. In some embodiments, a cell can comprise a cell derived from an animal. In some embodiments, a cell can be derived from a mammal, a bird, a reptile, an amphibian, a fish, an invertebrate, or any combination thereof. In some embodiments, a cell can be obtained from a biopsy. In some embodiments, a cell can be immortalized. In some embodiments, a synthetic leather can comprise an engineered cell from any cell type described herein and can comprise a molecular switch.

In some embodiments, a synthetic leather can comprise a cell derived from an animal. In some embodiments, a synthetic leather can comprise a cell derived from a mammal. In some embodiments, a cell derived from a mammal can comprise a mammalian cell. In some embodiments, a mammal can comprise a non-human mammal. In some embodiments, a non-human mammal can be an antelope, a bear, a beaver, a bison, a boar, a camel, a caribou, a cat, a cattle, a deer, a dog, an elephant, an elk, a fox, a giraffe, a goat, a hare, a horse, an ibex, a kangaroo, a lion, a llama, a lynx, a mink, a moose, an oxen, a peccary, a pig, a rabbit, a rhino, a seal, a sheep, a lamb, a squirrel, a tiger, a whale, a wolf, a yak, or a zebra. In some embodiments, an animal can be a primate, a bovine, an ovine, a porcine, an equine, a canine, a feline, a rodent, a lagomorph, a fish, a bird or a reptile. In some embodiments, an animal can comprise a reptile. In some embodiments, a reptile can comprise a crocodile, an alligator, or a snake. In some embodiments, a mammal can be a human. In some embodiments a human can be a celebrity. As used herein, the term “celebrity” can be defined as a person that has come into a community attention by way of notoriety or general fame of previous activities. A “celebrity” can be associated with industries including but not limited to professional and amateur sports, entertainment, music, motion picture, business, print and electronic media, politics, and the like.

In some embodiments, a synthetic leather can comprise cells derived from other species. In some embodiments, a cell can be derived from a bird. In some embodiments, a bird can comprise a chicken, a duck, an emu, a goose, a grouse, an ostrich, a pheasant, a pigeon, a quail, or a turkey. In some embodiments, a cell can be derived from a reptile such as a turtle, a snake, a lizard, an amphibian, a crocodile, or an alligator. In some embodiments, a cell can be derived from an amphibian. In some embodiments, an amphibian can comprise a frog, a toad, a salamander, or a newt. In some embodiments, a cell can be derived from a fish. In some embodiments, a fish can comprise an anchovy, a bass, a catfish, a carp, a cod, an eel, a flounder, a fugu, a grouper, a haddock, a halibut, a herring, a mackerel, a mahi-mahi, a manta ray, a marlin, an orange roughy, a perch, a pike, a pollock, a salmon, a sardine, a shark, a snapper, a sole, a stingray, a swordfish, a tilapia, a trout, a tuna, or a walleye.

In some embodiments, a cell in a synthetic leather can be derived from a same species. In some embodiments, all cells in a synthetic leather can be bovine cells. In some embodiments, a synthetic leather can comprise a cell derived from multiple species. In some embodiments, a synthetic leather can comprise a bovine cell and an alligator cell. In some embodiments, a synthetic leather can comprise a cell derived from at least 2, 3, 4, 5, 6, 7, 8, or 10 species.

In some embodiments, a progenitor of a cell in a synthetic leather can also be derived from a source described herein. In some embodiments, an engineered cell can comprise a molecular switch, a somatic cell, a primary cell used in a synthetic cell, a dermal layer cell, an epidermal layer cell, or any cells in a synthetic cell and their progenitors thereof can be derived from sources described herein. In some embodiments, a somatic cell can be reprogramed to an iPSC.

In some embodiments, a synthetic leather can comprise one or more layered structures. In some embodiments, a layered structure can be formed by placing a first type of layer upon a second type of layer. In some embodiments, a first type of layer and a second type of layer can be a same or different. In some embodiments, a layered structure can be formed by placing an epidermal layer upon a dermal layer. In some embodiments, a layered structure can be formed by placing an epidermal layer upon a dermal layer, with a basement membrane substitute in between. In some embodiments, a layered structure can comprise multiple layers of dermis.

In some embodiments, a layered structure can comprise two or more layers. In some embodiments, a layered structure can comprise at least 2, 3, 4, 5 6, 7, 8, 9, 10, 15, 20, 30, 40, 50, 60, 70, 80, 90, 100, 500, or 1000 layers. In some embodiments, a layered structure can comprise at least 2, 3, 4, 5 6, 7, 8, 9, 10, 15, 20, 30, 40, 50, 60, 70, 80, 90, 100, 500, or 1000 first type of layers, and at least 2, 3, 4, 5 6, 7, 8, 9, 10, 15, 20, 30, 40, or 50 second type of layers. In some embodiments, a layered structure can comprise at least 2, 3, 4, 5 6, 7, 8, 9, 10, 15, 20, 30, 40, 50, 60, 70, 80, 90, 100, 500, or 1000 dermal layers, and at least 2, 3, 4, 5 6, 7, 8, 9, 10, 15, 20, 30, 40, 50, 60, 70, 80, 90, 100, 500, or 1000 layers of epidermal layers.

In some embodiments, a layered structure can comprise one or more types of cell described herein. In some embodiments, a layered structure can comprise cells in a dermal layer, such as fibroblasts, cells in an epidermal layer, or any combination thereof. In some embodiments, a cell in an epidermal layer can comprise a keratinocyte. In some embodiments, a layered structure can comprise an engineered cell with a switch. In some embodiments, a layered structure can comprise an immortalized cell with an exogenous molecular switch.

In some embodiments, a layered structure can have a thickness from about 0.001 mm to about 100 mm. In some embodiments, a layered structure can have a thickness from about 0.005 mm to about 50 mm, from about 0.005 to about 10, from about 0.01 mm to about 10 mm, from about 0.02 to about 5 mm, from about 0.05 to about 5 mm, from about 0.1 to about 5 mm, from about 0.1 to about 2 mm, from about 0.1 to about 1 mm, or from about 0.1 to about 0.5 mm. In some embodiments, a thickness of a layered structure can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, 10 mm, 20 mm, 40 mm, 60 mm, 80 mm, or 100 mm. In some embodiments, a thickness of a layered structure can be at most 100 mm, 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a layered structure can have a thickness of at least about 100, 200, 300, 400, 500, 600, 700, 800 mm.

In some embodiments, a length of a layered structure can be engineered to fit a function or use of a synthetic leather. In some embodiments, a layered structure can have a length from about 0.01 mm to about 50 m. In some embodiments, a layered structure can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a layered structure can have a length from about 0.02 mm to 5 mm. In some embodiments, a layered structure can have a length from about 0.1 mm to 0.5 mm. In some embodiments, a layered structure can have a length from about 0.2 mm to 0.5 mm. In some embodiments, a length of a layered structure can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a length of a layered structure can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a layered structure can have a length of at least about 50, 60, 70, 80, 90, 100, 200, 300, 400, 500, 700, 1000 mm. In some embodiments, a layered structure can have a length of at least about 50, 60, 70, 80, 90, 100, 200, 300, 400, 500, 600, 700 cm. In some embodiments, a layered structure can have a length of at least about 50, 60, 70, 80, 90, 100, 200, 300, 400 m.

In some embodiments, a width of a layered structure can be engineered to fit a function or use of a synthetic leather. A layered structure can have a width from about 0.01 mm to about 50 m. For example, a layered structure can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a layered structure can have a width from about 0.02 mm to 5 mm. For example, a layered structure can have a width from about 0.1 mm to 0.5 mm. For example, a layered structure can have a width from about 0.2 mm to 0.5 mm. In some embodiments, a width of a layered structure can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a width of a layered structure can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a layered structure can have a width of at least about 50, 60, 70, 80, 90, 100, 200, 300, 400, 500, 700, 1000 mm. In some embodiments, a layered structure can have a width of at least about 50, 60, 70, 80, 90, 100, 200, 300, 400, 500, 600, 700 cm. In some embodiments, a layered structure can have a width of at least about 50, 60, 70, 80, 90, 100, 200, 300, 400 m.

A layered structure can comprise fibroblasts and keratinocytes at any ratio of at least about 50:1, 40:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:10, or 1:100. In some embodiments, a ratio of fibroblasts to keratinocytes can be from about 20:1 to about 3:1, from about 20:1 to about 4:1, from about 20:1 to about 5:1, from about 20:1 to about 10:1, or from about 20:1 to about 15:1. In some embodiments, a layered structure can comprise any cell disclosed herein. In some embodiments, a layered structure can comprise any engineered cell disclosed herein.

A layered structure can comprise fibroblasts and melanocytes at any ratio of at least about 50:1, 40:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:10, or 1:100. In some embodiments, a ratio of fibroblasts to melanocyte can be from about 20:1 to about 3:1, from about 20:1 to about 4:1, from about 20:1 to about 5:1, from about 20:1 to about 10:1, or from about 20:1 to about 15:1.

A layered structure can comprise keratinocytes and melanocytes at any ratio of at least about 50:1, 40:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:10, or 1:100. In some embodiments, a ratio of keratinocytes to melanocyte can be from about 20:1 to about 3:1, from about 20:1 to about 4:1, from about 20:1 to about 5:1, from about 20:1 to about 10:1, or from about 20:1 to about 15:1.

One type of cells in a layered structure can comprise at most 99%, 95%, 90%, 85%, 80%, 75%, 70%, 65%, 60%, 55%, 50%, 45%, 40%, 35%, 30%, 25%, 20%, 10%, 5%, or 1% of a total cell population in a layered structure. One type of cells in a layered structure can comprise about at least 1%, 5%, 10%, 20%, 30%, 40%, 50%, 60%, 65%, 70%, 75%, 80%, 85%, 90%, or 95% of a total cell population in a layered structure. For example, fibroblasts in a layered structure can comprise about at least 5%, 10%, 20%, 30%, 40%, 50%, 60%, 65%, 70%, 75%, 80%, 85%, 90%, or 95% of a total cell population in a layered structure.

Disclosed herein in some embodiments, is a synthetic leather. Disclosed herein in some embodiments, are methods of forming a synthetic leather. In some embodiments, a synthetic leather can comprise at least a portion of a layer of cells, an at least partially decellularized layer of cells, one or more layered structures, one or more at least partially decellularized layered structures, or any combination thereof. In some embodiments, a synthetic leather can be formed by one or more layered structures, a dermis, or an epidermis. In some embodiments, a synthetic leather can be formed by at least 1, 2, 3, 4, 5, 6, 7, 8, 9, 10, 15, 20, 30, 40, 50, 60, 70, 80, 90, or 100 layered structures.

In some embodiments, a synthetic leather can be of various thickness. In some embodiments, a synthetic leather can have a thickness resembling a natural leather. In some embodiments, a synthetic leather can have a thickness from about 0.001 mm to about 100 mm. For example, a layered structure can have a thickness from about 0.005 mm to about 50 mm, from about 0.005 to about 10, from about 0.01 mm to about 10 mm, from about 0.1 to about 5 mm, from about 0.5 mm to about 5 mm, from about 0.5 mm to about 3 mm, from about 0.8 mm to about 3 mm, from about 0.8 mm to about 2 mm, from about 0.8 mm to about 1.8 mm, from about 0.8 mm to about 1.6 mm, from about 0.9 mm to about 1.4 mm, from about 1 mm to about 1.5 mm, from about 1 mm to about 1.4 mm, or from about 1 mm to about 1.3 mm. In some embodiments, a thickness of a synthetic leather can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, 10 mm, 20 mm, 40 mm, 60 mm, 80 mm, or 100 mm. In some embodiments, a thickness of a synthetic leather can be at most 100 mm, 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a thickness of a synthetic leather can be about 1.2 mm.

A synthetic leather can have a length from about 0.01 mm to about 50 m. For example, a synthetic leather can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a synthetic leather can have a length from about 0.02 mm to 5 mm. For example, a synthetic leather can have a length from about 0.1 mm to 0.5 mm. For example, a synthetic leather can have a length from about 0.2 mm to 0.5 mm. In some embodiments, a length of a synthetic leather can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a length of a synthetic leather can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a synthetic leather can have a length of at least about 50, 60, 70, 80, 90, 100, 200, 300, 400, 500, 700, 1000 mm. In some embodiments, a synthetic leather can have a length of at least about 50, 60, 70, 80, 90, 100, 200, 300, 400, 500, 600, 700 cm. In some embodiments, a synthetic leather can have a length of at least about 50, 60, 70, 80, 90, 100, 200, 300, 400 m.

A synthetic leather can have a width from about 0.01 mm to about 50 m. For example, a synthetic leather can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a synthetic leather can have a width from about 0.02 mm to 5 mm. For example, a synthetic leather can have a width from about 0.1 mm to 0.5 mm. For example, a synthetic leather can have a width from about 0.2 mm to 0.5 mm. In some embodiments, a width of a synthetic leather can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a width of a synthetic leather can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a synthetic leather can have a width of at least about 50, 60, 70, 80, 90, 100, 200, 300, 400, 500, 700, 1000 mm. In some embodiments, a synthetic leather can have a width of at least about 50, 60, 70, 80, 90, 100, 200, 300, 400, 500, 600, 700 cm. In some embodiments, a synthetic leather can have a width of at least about 50, 60, 70, 80, 90, 100, 200, 300, 400 m.

In some embodiments, a synthetic leather can comprise a biofabricated material. In some embodiments, a leather can comprise a biofabricated material. In some embodiments, a biofabricated material can comprise a cell, an engineered cell, or a tissue disclosed herein. In some embodiments, a biofabricated material can comprise an engineered cell described herein. In some embodiments, a biofabricated material can comprise zonal properties. A zonal property can comprise one or more of zones in a biofabricated material. In some embodiments, a zone can possess one or more properties that may be different than one or more zones adjacent thereto. In some embodiments, properties that may be varied from zone to zone comprise: color, breathability, stretchability, tear strength, softness, rigidity, abrasion resistance, heat transfer to enable warming or cooling, electromagnetic, luminescence, reflectance, antimicrobial, antifungal, antibacterial, strength, fragrance and combinations thereof.

A synthetic leather can further comprise a basement membrane substitute. A basement membrane substitute can be between two cell layers, e.g., between a dermal layer and an epidermal layer. A basement membrane substitute can be a dermo-epidermal junction similar to that which exists in vivo, from a structural point of view and/or from a biochemical point of view. From a biochemical point of view, a basement membrane substitute can comprise components of a basal membrane, of a lamina densa, of a lamina lucida and of a sub-basal zone, such as, collagen IV, collagen VII, laminin 5, entactin fibronectin, or any combination thereof.

A basement membrane substitute in a synthetic leather can be urinary basement membrane (UBM), liver basement membrane (LBM), amnion, chorion, allograft pericardium, allograft acellular dermis, amniotic membrane, Wharton's jelly, or any combination thereof. For example, a basement membrane substitute can be a dried acellular amniotic membrane. In certain cases, a basement membrane substitute can be a polymer, e.g., a nanopolymer. For example, a basement membrane substitute can be nano-fibrous poly hydroxybutyrate-cohydroxyvalerate (PHBV), as described by Bye et al., Journal of Biomaterials and Tissue Engineering Vol. 4, 1-7, 2014.

In some embodiments, a cell, a cell layer, a layered structure, or a synthetic leather can be seeded onto a scaffold. In some embodiments, a scaffold can comprise a substrate. In some embodiments, a scaffold can provide a certain firmness (e.g., resistance to tearing), elasticity, or both. In some embodiments, a synthetic leather can comprise a part of or an entire scaffold. In some embodiments, a synthetic leather may not comprise a scaffold. In some embodiments, after assisting a formation of a layer in a synthetic leather, a scaffold can be removed from a final synthetic leather product. In some embodiments, a scaffold comprised in a synthetic leather may be degraded after a period of time. In some embodiments, a scaffold can be degradable, biodegradable, bioabsorbable, resorbable, or any combination thereof.

In some embodiments, a scaffold can be made of natural materials, synthetic materials, or any combination thereof. In some embodiments, a scaffold can comprise a substrate. In some embodiments, a substrate can comprise a substrate for cell growth. In some embodiments, a scaffold can be formed using a net made of a bioabsorbable synthetic polymer. In some embodiments, a scaffold can be formed by attaching a nylon net to a silicon film. In some embodiments, a scaffold can comprise a two-layered structure of a collagen sponge and a silicon sheet. In some embodiments, a scaffold can be formed using an atelocollagen sponge. In some embodiments, a scaffold can be made into a sheet. In some embodiments, a scaffold can be formed by matching collagen sponges having different pore sizes. In some embodiments, acellular dermal matrices (ADM) can be formed using fibrin glue, allogeneic skin, or a combination thereof that has been at least partially decellularized.

In some embodiments, a scaffold can comprise natural substances such as collagen (e.g., collagen matrix), natural adhesive (e.g., fibrin glue, cold glues, animal glue, blood albumen glue, casein glue, or vegetable glues such as starch and dextrin glues). In some embodiments, a scaffold can comprise a polylactide, a polyglycolide, a polycaprolactone, a hydrogel, or any combination thereof. In some embodiments, a scaffold can comprise a silk. In some embodiments, a scaffold can be made of a silk. In some embodiments, a scaffold can comprise a silk fibroin, a cellulose, a cotton, an acetate, an acrylic, a latex fiber, a linen, a nylon, a rayon, a velvet, a modacrylic, an olefin polyester, a saran, a vinyon, a wool, a jute, a hemp, a bamboo, a flax or a combination thereof. In some embodiments, a scaffold can comprise a fiber. In some embodiments, a fiber can be a fiber of a silk, a cotton, a wool, a linen, a cellulose extracted in particular from a wood, a vegetable, an algae, a polyamide, a modified cellulose, a poly-p-phenyleneterephthalamide, an acrylic fiber, for example those of polymethyl methacrylate or of poly-2-hydroxyethyl methacrylate, fibers of polyolefin for example fibers of polyethylene or polypropylene, glass, silica, aramid, carbon, for example in a form of a graphite, a poly(tetrafluoroethylene), an insoluble collagen, a polyester, a polyvinyl chloride, a polyvinylidene chloride, a polyvinyl alcohol, a polyacrylonitrile, a chitosan, a polyurethane, a poly(urethane-urea), a polyethylene phthalate, and fibers formed from a blend of polymers such as those mentioned above, such as polyamide/polyester fibers, or any combination thereof. In some embodiments, a modified cellulose can comprise a rayon, a viscose, an acetate, a rayon acetate, or any combination thereof.

In some embodiments, a scaffold can comprise a polymer. In some embodiments, a polymer can comprise a biopolymer. In some embodiments, a biopolymer can include but may not be limited to a chitin, a chitosan, an elastin, a collagen, a keratin or a polyhydroxyalkanoate. In some embodiments, a polymer can be biodegradable, biostable, or a combination thereof. In some embodiments, a polymer in a scaffold can be a natural polymer. In some embodiments, an exemplary natural polymer can include a polysaccharide such as an alginate, a cellulose, a dextran, a pullane, a polyhyaluronic acid, a chitin, a poly(3-hydroxyalkanoate), a poly(3-hydroxyoctanoate), a poly(3-hydroxyfatty acid), or any combination thereof. In some embodiments, a polymer can comprise a polyethylene (PE), a polypropylene (PP), a Polyethylene terephthalate (PET), a Polyamide 6,6 (PA 6,6), a Polyamide 11 (PA 11), a Polyvinylidene fluoride (PVDF), a Polyethylene furanoate (PEF), a Polyurethane (PU), a Polyhydroxyalkanoate (PHA), a Polyhydroxybutyrate (PHB), a Poly(3-hydroxybutyrate-co-3-hydroxyvalerate) (PHBV), a Polylactic acid (PLA), a Polycaprolactone (PCL), a Polybutylene succinate (PBS), a Poly(glycolic) acid (PGA), a Poly(lactic-co-glycolic acid (PLGA), a Polyvinyl Alcohol (PVOH), an Alginate, a Copolymer PEGylated fibrin (P-fibrin), a Poly(glycerol sebacate) (PGS), a poly(L-lactic acid) (PLLA), a Poly(lactic-coglycolic acid) (PLGA), a Poly-D,L-lactic acid/polyethylene glycol/poly-D,L-lactic acid (PDLLA-PEG), a hyaluronic acid (HA), or any combination thereof. In some embodiments, a scaffold can also comprise a chemical derivative of a natural polymer. In some embodiments, a chemical derivative can include a substitution and/or an addition of a chemical group such as an alkyl, an alkylene, a hydroxylation, an oxidation, another chemical modification, or any combination thereof. In some embodiments, a natural polymer can also be selected from a protein such as collagen, zein, casein, gelatin, gluten, and serum albumen. In some embodiments, a polymer in a scaffold can be biodegradable synthetic polymers, including poly alpha-hydroxy acids such as poly L-lactic acid (PLA), polyglycolic acid (PGA) or copolymers thereof (e.g., poly D,L-lactic co-glycolic acid (PLGA)), and hyaluronic acid.

In some embodiments, a scaffold can be bioabsorbable. In some embodiments, a bioabsorbable scaffold can be a non-cytotoxic structure or substance that may be capable of containing or supporting living cells and holding them in a desired configuration for a period of time. In some embodiments, the term “bioabsorbable” can refer to any material a body can break down into non-toxic by-products that can be excreted from a body or metabolized therein. In some embodiments, an exemplary bioabsorbable material for a scaffold can include, a poly(lactic acid), a poly(glycolic acid), a poly(trimethylene carbonate), a poly(dimethyltrimethylene carbonate), a poly(amino acids)s, a tyrosine-derived poly(carbonates)s, a poly(carbonates)s, a poly(caprolactone), a poly(para-dioxanone), a poly(esters)s, a poly(ester-amides)s, a poly(anhydrides)s, a poly(ortho esters)s, a collagen, a gelatin, a serum albumin, a protein, a polysaccharide, a mucopolysaccharide, a carbohydrate, a glycosaminoglycan, a poly(ethylene glycols), a poly(propylene glycols), a poly(acrylate esters), a poly(methacrylate esters), a poly(vinyl alcohol), a hyaluronic acid, a chondroitin sulfate, a heparin, a dermatan sulfate, a versican, a copolymer, a blend of polymers, a mixture of polymers, an oligomer containing bioabsorbable linkages, or any combination thereof.

In some embodiments, a scaffold can be a mesh. In some embodiments, a mesh can be a network of material (e.g. threads, strings, strands, fibers, or any combination thereof) that can be connected by weaving or otherwise. In some embodiments, a mesh can comprise a material that can be artificial, biological or any combination thereof. In some embodiments, a mesh can have pores that can be regular or irregular in size, regular or irregular in shape, regular or irregular in pattern, or any combination thereof. In some embodiments, a mesh can be two-dimensional or three-dimensional. In some embodiments, a mesh can have a pore from about: 10 nm to 10 cm in diameter, spacing, or any combination thereof. In some embodiments, a pore size can be generally about: 10 nm, 20 nm, 50 nm, 100 nm, 200 nm, 300 nm, 400 nm, 500 nm, 600 nm, 700 nm, 800 nm, 900 nm, 1 mm, 1.1 mm, 1.2 mm, 1.3 mm, 1.4 mm, 1.5 mm, 1.6 mm, 1.7 mm, 1.8 mm, 1.9 mm, 2 mm, 3 mm, 4 mm, 5 mm, 6 mm, 7 mm, 8 mm, 9 mm, 10 mm, 12 mm, 15 mm, 2 cm, 2.5 cm, 3 cm, 4 cm, 5 cm, 6 cm, 7 cm, 8 cm, 9 cm, or 10 cm. A mesh can have a diameter of about: 20 nm, 50 nm, 100 nm, 200 nm, 300 nm, 400 nm, 500 nm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 3 mm, 4 mm, 5 mm, 6 mm, 7 mm, 8 mm, 9 mm, 10 mm, 12 mm, 15 mm, 2 cm, 2.5 cm, 3 cm, 4 cm, 5 cm, 6 cm, 7 cm, 8 cm, 9 cm, or 10 cm. In some embodiments, strands (e.g., fiber, threads, webs, etc.) or material forming a mesh can have diameters of from about 50 nm to about 10 mm in diameter. In some embodiments, a mesh may not be a scaffold.

In some embodiments, a scaffold can be a supporting structure for cell proliferation. In some embodiments, a scaffold can be permeable to a fluid, a nutrient, such that a cell culture medium can contact a surface of a cell layer.

In some embodiments, a scaffold can comprise a three-dimensional structure. In some embodiments, a scaffold can be porous. In some embodiments, a cell can be seeded within a scaffold. In some embodiments, a scaffold can be of various thicknesses. In some embodiments, a scaffold can have a thickness that may be suitable for forming a cell layer. In some embodiments, a scaffold can have a thickness from about 0.1 mm to about 10 mm, such as from about 0.1 mm to about 5 mm, from about 0.1 mm to about 4 mm, from about 0.1 mm to about 3 mm, from about 0.1 mm to about 2 mm, to about 0.1 mm to about 1 mm, from about 0.2 mm to about 1 mm, from about 0.3 mm to about 1 mm, from about 0.4 mm to about 1 mm, from about 0.5 mm to about 1 mm, from 0.3 mm to about 1.5 mm, from about 0.4 mm to about 1.2 mm, from about 0.6 mm to about 1.2 mm, or from about 0.7 mm to about 1.5 mm. In some embodiments, a scaffold can have a thickness from about 0.5 mm to 1 mm. In some embodiments, a scaffold can be at least 0.1 mm, 0.2 mm, 0.3 mm, 0.4 mm, 0.5 mm, 0.8 mm, 1 mm, 2 mm, 3 mm, 4 mm, or 5 mm thick. In some embodiments, a scaffold can be at most 0.5 mm, 0.8 mm, 1 mm, 2 mm, 3 mm, 4 mm, 5 mm, 6 mm, 7 mm, 8 mm, 9 mm, or 10 mm thick. In some embodiments, a scaffold can have a length and/or a width of a cell layer to be placed and/or grown upon a scaffold. In some embodiments, a scaffold can have a length and/or a width of a cell layer described herein. In some embodiments, a scaffold can comprise a pore size of less than 1 nanometer. In some embodiments, a scaffold can comprise a pore size of greater than 1 nanometer. In some embodiments, a scaffold can comprise a pore size of between 10 μm and 900 μm.

In some embodiments, a cell layer may not form on a scaffold. In some embodiments, a dermal layer may not form on a scaffold (e.g., collagen matrix). In some embodiments, a synthetic leather does not comprise a scaffold.

In some embodiments, a synthetic leather can comprise one or more pigments. In some embodiments, one or more layer structures of a synthetic leather can be pigmented. In some embodiments, a pigment in a synthetic leather can be a natural pigment produced in cells forming a synthetic leather. In some embodiments, a pigment can comprise melanin, including eumelanin (e.g., brown eumelanin and black eumelanin), pheomelanin, neuromelanin, or any combination thereof. In some embodiments, a pigment in a synthetic leather can be an exogenous pigment, such as a leather pigment dye.

In some embodiments, a synthetic leather can comprise collagen. In some embodiments, a collagen can refer to any member of a family of at least 28 distinct collagen types. In some embodiments, a collagen can be characterized by a repeating triplet of amino acids, -(Gly-X-Y)n-, so that approximately one-third of amino acid residues in a collagen can be glycine. In some embodiments, X can be proline and Y can be hydroxyproline. In some embodiments, a structure of a collagen can have twined triple units of peptide chains of differing lengths. In some embodiments, a synthetic leather can comprise collagen from one or more species. In some embodiments, a synthetic leather can comprise collagen from different animals. In some embodiments, a different animal can produce different amino acid compositions of a collagen, which can result in different properties (and differences in a resulting leather). In some embodiments, a collagen fiber monomer can be produced from alpha-chains of about 1050 amino acids long, so that a triple helix takes a form of a rod of about 300 nm long, with a diameter of about 1.5 nm.

In some embodiments, a synthetic leather can comprise one or more types of collagen. In some embodiments, an engineered cell described herein can secrete collagen. In some embodiments, a collagen comprised in a synthetic leather can include a fibrillary collagen, a non-fibrillar collagen, or a combination thereof. In some embodiments, a fibrillary collagen can include type I, type II, type III, type V, type XI collagens, or any combination thereof. In some embodiments, a non-fibrillar collagen can include a fibril associated collagen with interrupted triple helices, short chain collagens, basement membrane collagens, Multiplexin (Multiple Triple Helix domains with Interruptions), MACIT collagens (Membrane Associated Collagens with Interrupted Triple Helices), or any combination thereof. In some embodiments, a fibril associated collagen with interrupted triple helices can comprise a type IX, a type XII, a type XIV, a type XVI, a type XIX collagen, or any combination thereof. In some embodiments, short chain collagens can comprise a type VIII, a type X collagen, or any combination thereof. In some embodiments, a basement membrane collagen can comprise a type IV collagen. In some embodiments, a multiplexin can comprise a Type XV, a type XVIII collagen, or a combination thereof. In some embodiments, a MACIT collagen can comprise a Type XIII, a type XVII collagen, or a combination thereof.

In some embodiments, a collagen can be comprised in one or more parts of a synthetic leather. In some embodiments, one or more dermal layers, one or more epidermal layers, or a combination thereof can comprise a collagen as disclosed herein. In some embodiments, one or more layered structures in a synthetic leather can comprise a collagen as disclosed herein. In some embodiments, when part of a synthetic leather is removed during a process, a collagen can also be comprised in a removed product.

In some embodiments, a collagen in a synthetic leather can be from one or more sources. In some embodiments, a collagen can be produced by a collagen producing cell in a synthetic leather. In some embodiments, a collagen can be separately added to a leather. In some embodiments, a synthetic leather can comprise a collagen produced by a collagen producing cell and a collagen separately added.

In some embodiments, at least part of a collagen in a synthetic leather can be produced by a collagen producing cell. In some embodiments, a method of forming a synthetic leather can comprise using a collagen producing cell. In some embodiments, a synthetic leather can comprise a collagen producing cell. In some embodiments, a collagen producing cell can include an engineered cell, an immortalized cell, an epithelial cell, a fibroblast, a keratinocyte, a corneocyte, a melanocyte, a Langerhans cell, a basal cell, a smooth muscle cell, or a combination thereof. In some embodiments, an epithelial cell can comprise a squamous cell, a cuboidal cell, a columnar cell, a basal cell, or any combination thereof. In some embodiments, a fibroblast can include a dermal fibroblast. In some embodiments, a keratinocyte can include an epithelial keratinocyte, a basal keratinocyte, a proliferating basal keratinocyte, a differentiated suprabasal keratinocyte, or any combination thereof. In some embodiments, a collagen in a synthetic leather can be produced by one or more types of collagen-producing cells.

In some embodiments, a collagen production can be induced in a cell by adding ascorbic acid, an analog of ascorbic acid, a salt thereof, or any combination thereof at a concentration of about 1 mM (millimolar) to about 5M. In some embodiments, a collagen fibrillation can be induced by adding a salt or a combination of salts, for example, a salt or combination of salts can include: Na₃PO₄, K₃PO₄, KCl, and NaCl. In some embodiments, a salt concentration of each salt can be from about 10 mM to about 5M.

In some embodiments, a cell can be genetically engineered to comprise a gene for a collagen or a plurality of genes for collagens. In some embodiments, a genetically engineered cell can include, an epithelial cell, a fibroblast, a keratinocyte, a corneocyte, a melanocyte, a Langerhans cell, a basal cell, a smooth muscle cell, an immortalized cell, a cell with a molecular switch or a combination thereof. In some embodiments, an epithelial cell can include a squamous cell, a cuboidal cell, a columnar cell, a basal cell, or a combination thereof. In some embodiments, a fibroblast can include a dermal fibroblast. In some embodiments, a keratinocyte can include an epithelial keratinocyte, a basal keratinocyte, a proliferating basal keratinocyte, a differentiated suprabasal keratinocyte, or a combination thereof. In some embodiments, a collagen gene can comprise P4HA, P4HB, COL1A1, COL1A2, COL2A1, COL3A1 or any combination thereof. In some embodiments, a collagen gene can have an altered promotor that can change an expression of a collagen gene, e.g., an increase or a decrease. In some embodiments, a collagen gene can be placed downstream of an engineered expression system. A collagen gene can be from a human, a mammal, a bird, a reptile, an amphibian, a fish, an invertebrate, or any combination thereof.

In some embodiments, a synthetic leather can further comprise one or more additives. Such additives can enhance a commercial appeal (e.g., appearance, color, or odor). In some embodiments, an exemplary additive can comprise a mineral, a fiber, a fatty acid, an amino acid, a protein, or any combination thereof. In some embodiments, an additive can be an odorant, a dye, a stain, a resin, a polymer, or any combination thereof.

In some embodiments, an additive can comprise a matrix protein, a proteoglycan, an antioxidant, a perfluorocarbon, a hormone, a growth factor, or any combination thereof. In some embodiments, a growth factor can be a protein, a polypeptide, or a complex of polypeptides, including cytokines (e.g., that can be produced by a cell and which can affect itself and/or a variety of other neighboring or distant cells). In some embodiments, a growth factor can affect a growth and/or differentiation of specific types of cells, either developmentally or in response to a multitude of physiological or environmental stimuli. In some embodiments, a growth factor can comprise a hormone. In some embodiments, a growth factor can comprise an insulin, an insulin-like growth factor (IGF), a nerve growth factor (NGF), a vascular endothelial growth factor (VEGF), a keratinocyte growth factor (KGF), a fibroblast growth factor (FGFs), a basic FGF (bFGF), a platelet-derived growth factor (PDGFs), a PDGF-AA, a PDGF-AB, a hepatocyte growth factor (HGF), a transforming growth factor alpha (TGF-alpha), a transforming growth factor beta (TGF-β), a TGFpi, a TGFP3, an epidermal growth factor (EGF), a granulocyte-macrophage colony-stimulating factor (GM-CSF), a granulocyte colony-stimulating factor (G-CSF), an interleukin-6 (IL-6), an IL-8, or any combination thereof. In some embodiments, a polypeptide or a molecule (e.g., healing agents; enzymes such as matrix-degrading enzymes and matrix-degrading enzyme inhibitors (e.g., TIMPs), antibiotics, and antimycotics) can also be added to a synthetic leather.

In some embodiments, an additive can comprise a preservative In some embodiments, a preservative can comprise an antimicrobial preservative such as a calcium propionate, a sodium nitrate, a sodium nitrite, a sulfite (e.g., sulfur dioxide, sodium bisulfate, potassium hydrogen sulfite, etc.), a disodium ethylenediaminetetraacetic acid (EDTA), an antioxidant such as butylated hydroxyanisole (BHA) and a butylated hydroxytoluene (BHT).

In some embodiments, a synthetic leather can comprise an extracellular matrix or connective tissue. In some embodiments, a synthetic leather can further comprise a collagen, a keratin, an elastin, a gelatin, a proteoglycan, a dermatan sulfate proteoglycan, a glycosoaminoglycan, a fibronectin, a laminin, a dermatopontin, a lipid, a fatty acid, a carbohydrate, or any combination thereof.

In some embodiments, a synthetic leather can be patterned. For example, a synthetic leather may be patterned after a skin pattern of an animal selected from antelope, bear, beaver, bison, boar, camel, caribou, cat, cattle, deer, dog, elephant, elk, fox, giraffe, goat, hare, horse, ibex, kangaroo, lion, llama, lynx, mink, moose, oxen, peccary, pig, rabbit, seal, sheep, squirrel, tiger, whale, wolf, yak, zebra, turtle, snake, crocodile, alligator, dinosaur, frog, toad, salamander, newt, chicken, duck, emu, goose, grouse, ostrich, pheasant, pigeon, quail, turkey, anchovy, bass, catfish, carp, cod, eel, flounder, fugu, grouper, haddock, halibut, herring, mackerel, mahi, manta ray, marlin, orange roughy, perch, pike, pollock, salmon, sardine, shark, snapper, sole, stingray, swordfish, tilapia, trout, tuna, walleye, and a combination thereof. In some embodiments, a pattern can be a skin pattern of a fantasy animal selected from dragon, unicorn, griffin, siren, phoenix, sphinx, Cyclops, satyr, Medusa, Pegasus, Cerberus, Typhoeus, gorgon, Charybdis, empusa, chimera, Minotaur, Cetus, hydra, centaur, fairy, mermaid, Loch Ness monster, Sasquatch, thunderbird, yeti, chupacabra, and a combination thereof.

In some embodiments, a synthetic leather can be made to resemble traditional animal skin, hide, or leather products and design parameters (e.g., cell types, additives, size, shape). In some embodiments, a synthetic leather can comprise a cell layer characterized by a composition that may be substantially similar to traditional animal skin, hide, or leather products. For example, such layer can be characterized by a composition that may be substantially about 60% to 80% aqueous fluid, about 14%-35% protein, about 1%-25% fat. In some embodiments, keratinocytes of a cell layer can be aligned. In some embodiments, a keratinocyte can be aligned by application of an electrical field. For example, keratinocytes can be aligned by application of a mechanical stimulus, such as cyclical stretching and relaxing a substratum. In some embodiments, aligned (e.g., electro-oriented and mechano-oriented) keratinocytes can have substantially a same orientation with regard to each other as can be found in many animal skin tissues.

In some embodiments, a synthetic leather herein can be at least a portion of a leather article. For example, a synthetic leather can be used as substitute of natural leather in a leather article. Exemplary leather articles include a watch strap, belt, suspender, packaging, shoe, boot, footwear, glove, clothing (e.g., tops, bottoms, and outerwear), luggage, bag (e.g., a handbag with or without shoulder strap), clutch, purse, coin purse, billfold, key pouch, credit card case, pen case, backpack, cases, wallet, saddle, harness, whip, travel goods (e.g., a trunk, suitcase, travel bag, beauty case, or a toilet kit), rucksacks, portfolio, document bag, briefcase, attaché case, pet article (e.g., a leash or collar), hunting and fishing article (e.g., a gun case, cutlery case, or a holster for firm arms), a stationary article (e.g., a writing pad, book cover, camera case, spectacle case, cigarette case, cigar case, jewel case, or a mobile phone holster), or a sport article (e.g., a ball such as basketball, soccer ball, or a football). For example, a leather article can be a watch wrap. For example, a leather article can be a belt. For example, a leather article can be a bag.

In some embodiments, a synthetic leather or portions thereof can also be used as a skin graft, e.g., an allograft or xenograft for transplanting to a subject. For example, a synthetic leather, dermal layer, epidermal layer and/or a layered structure can be a source of skin graft for allotransplant or xenotransplant. In some embodiments, a synthetic leather, dermal layer, epidermal layer and/or a layered structure can be produced with cells genetically modified to reduce immune-rejection in a recipient of a graft.

Disclosed herein in some embodiments, are methods for developing an engineered cell comprising a molecular switch. In some embodiments, making a synthetic leather can comprise forming an artificial dermal layer, forming an artificial epidermal layer, forming a tissue, or a combination thereof. In some embodiments, an artificial dermal layer, an artificial epidermal layer, a tissue, or a combination thereof can be at least partially decellularized. In some embodiments, an at least partial decellularizing can comprise contacting with a salt solution. In some embodiments, a method can further comprise tanning at least of a portion of an artificial dermal layer, an artificial epidermal layer, an artificial tissue or any combination thereof. In some embodiments, a cell in a synthetic leather, e.g., those in a tissue can comprise an immortalized cell with a molecular switch. In certain cases, a method can comprise placing a first cell layer (e.g., an epidermal layer) upon a second cell layer (e.g., a dermal layer) thereby forming a layered structure, and tanning at least a portion of a layered structure. In some embodiments, a method can further comprise removing at least a portion of a cell layer (e.g., an epidermal layer). In some embodiments, at least a portion of a cell layer can be removed prior to a tanning. In some embodiments, a method herein can further comprise creating a molecular switch and an immortalized cell.

Disclosed herein in some embodiments, are methods to make a molecular switch. In some embodiments, a molecular switch can be formed by genetically engineering a cell to contain exogenous DNA that can be programmed to elicit a phenotype after exposure to a stimulus. In some embodiments, a cell can be an animal cell, for example a bovine cell. In some embodiments, a cell can be a fibroblast or a stem cell. In some embodiments, a cell can be an immortalized cell. In some embodiments, a molecular switch can be introduced into a cell by transfection, transduction, electroporation or any combination thereof. In some embodiments, an exogenous DNA can be introduced by electroporation or by a viral vector into a host cell. In some embodiments, a molecular switch or a polynucleotide encoding a molecular switch can be introduced by a vector, wherein a vector can be a virus, a viral-like particle, an adeno-associated viral vector, a liposome, a nanoparticle, a plasmid, a linear dsDNA and a combination thereof. In some embodiments, a molecular switch can be in a genome or a molecular switch can be extrachromosomal, e.g., a plasmid.

In some embodiments, forming a molecular switch can include adding a gene, a promotor, a polynucleotide sequence, or any combination thereof, to a cell to drive a desired response to a stimulus. In some embodiments, a response can be a proliferation phenotype modification (e.g., immortalization), a reporter signal (e.g. expression of a fluorophore), a change in expression, a transcriptional change, or any combination thereof. In some embodiments, a response can be an increase or a decrease in gene expression. In some embodiments, a stimulus can be an environmental stimulus (e.g., a temperature). In some embodiments, a stimulus can be an antibiotic (e.g., tetracycline). In some embodiments, a molecular switch can be configured to cause a cell to grow anchorage independent, anchorage dependent, or any combination thereof. In some embodiments, a stimulus can be added to elicit a change from anchorage independent proliferation to anchorage dependent proliferation, or a stimulus can be added to elicit a change from anchorage dependent proliferation to anchorage independent proliferation. In some embodiments, a stimulus can elicit a removal or addition of a gene by a Cre-Lox system, a CRISPR system, or any combination thereof. In some embodiments, anchorage independent proliferation can be a result of a change in expression (e.g., an increase or decrease) of integrin-linked kinase (ILK), cyclin D1, Cdk4, ST6 N-Acetylgalactosaminide Alpha-2, 6-Sialytransferase 5 (ST6GALNAC5), or a combination thereof. In some embodiments, anchorage dependent proliferation can be a result by a change in expression of genes in a P53-mediated apoptosis pathway. In some embodiments, anchorage independent growth can be accomplished by modulation of an integrin signaling, a cell cycle, an apoptosis pathway (e.g., cloning a cell cycle gene into a molecular switch), or any combination thereof.

In some embodiments, an immortalized cell can comprise any cell. In some embodiments, an immortalized cell can be made from a fibroblast cell, a fat tissue derived cell, an umbilical cord cell, a keratinocyte, a corneocyte, a melanocyte, a Langerhans cell, a basal cell, an adipocyte, a smooth muscle cell, an epithelial cell, a cuboidal cell, a columnar cell, a collagen producing cell or any combination thereof. In some embodiments, an immortalized cell can be made from any cell. In some embodiments, an immortalized cell can be made from a primate cell, a bovine cell, an ovine cell, a porcine cell, an equine cell, a canine cell, a feline cell, a rodent cell, a bird cell, a marsupial, a reptile, a lagomorph animal cell or any combination thereof. In some embodiments, an immortalized cell can be made from a pluripotent stem cell, an induced pluripotent stem cell, a mesenchymal stem cell, or an embryonic stem cell. In some embodiments, an immortalized cell can have similar characteristics to any cell type described herein. In some embodiments, an immortalized cell can have a mutation. In some embodiments, an immortalized cell can be made from by transfecting, transducing, electroporation, or any combination thereof exogenous gene or exogenous genes. In some embodiments, a vector can carry an exogenous gene. In some embodiments, a vector can be a virus, a viral-like particle, an adeno-associated viral vector, a liposome, a nanoparticle, a plasmid, linear dsDNA and a combination thereof. In some embodiments, a vector can comprise a plasmid. In some embodiments, an exogenous gene can comprise hTERT, TERT, Bmi1, CcnD1, a mutant of Cdk4, Cdk4, TAg (SV40 large T), SV40, c-myc, H-ras, Ela, c-mMycER^{TAM}, E6, E7, HER-2, SRC, EGFR, Abl, Atk02, Aml1, Axl, Bcl, Dbl, EGFR, ERBB, Ets-1, Fins, Fos, Fps, Gli, Gsp, Her2, Hox11, Hst, 11-3, Jnt-2, Jun, Kit, KS3, K-SAM, Lbc, Lck, L-myc, Lyl-1, Lyt-10, Mas, MDM-2, Mll, Mos, Myb, Neu, N-Myc, Ost, Pax-5, Pim-1, PRAD-1, Ras-K, Ras-N, Ret, Ros, Ski, Sis, Set, Src, Tall, Tan1, Tiam1, Tsc2, Trk, or any combination thereof. In some embodiments, immortalization can be from an exogenous gene, which can be a fusion gene that can comprise one or more genes. An exogenous gene can integrate into a chromosome or be extrachromosomal. In some embodiments, immortalization can be from a protein product or a biologically active fragment thereof that can induce immortalization. In some embodiments, an immortalized cell can have an exogenous gene removed after cell divisions (e.g., by a Cre-LoxP system or a CRISPR system). In some embodiments, an exogenous gene can be from a human. In some embodiments, an exogenous gene can be from a mammal, a bird, a reptile, an amphibian, a fish, an invertebrate, a virus, a bacterium, or any combination thereof. In some embodiments, immortalization can be from a random mutation or a plurality of mutations. In some embodiments, a mutation can be generated by UV mutagenesis, chemical mutagenesis, or any combination thereof. In certain cases, immortalization can be from a targeted mutation, for example, a targeted mutation may be made by a CRISPR system. In some embodiments, a mutation can be in a cell cycle gene, an oncogene, a metabolic gene, or any combination thereof. In some embodiments, immortalization can be from a mutation in an oncogene, a cell cycle gene, a gene, a promotor region, an intragenic region, an intergenic region, or any combination thereof. In some embodiments, an immortalized cell can have increased or decreased expression of an oncogene or genes involved in a regulation of cell proliferation. In some embodiments, an immortalized cell can comprise a protein that acts through competitive inhibition. In some embodiments, competitive inhibition can comprise modifying an activity of a tumor suppressor gene, a cell cycle gene, or a combination thereof. In some embodiments, a Hayflick limit or Hayflick number can comprise a finite number of cell doublings which a primary cell can be grown to. In some embodiments, an immortalized cell can be grown past a Hayflick limit. In some embodiments, an immortalized cell can be grown past about 40 cell divisions, about 50 cell divisions, about 60 cell divisions, about 70 cell divisions, about 80 cell divisions, about 90 cell divisions, about 100 cell divisions, about 200 cell divisions, about 300 cell divisions, about 400 cell divisions, about 500 cell divisions, about 600 cell divisions, about 700 cell divisions, about 800 cell divisions, about 900 cell divisions, about 1000 cell divisions, about 10,000 cell divisions, or about 100,000 cell divisions. In some embodiments, an immortalized cell line can be grown enough to generate at least 1 million square feet per year of synthetic leather. In some embodiments, an immortalized cell can have a molecular switch. In some embodiments, an immortalized cell can be a conditional immortalized cell. A conditional immortalized cell can display an immortalized cellular phenotype under a certain environmental stimulus or a differentiated cellular phenotype under a different environmental stimulus.

A cell layer can be formed by preparing a plurality of multicellular bodies comprising one or more type of cells and arranging such multicellular bodies to form a cell layer. For example, a cell layer can be formed by adjacently arranging a plurality of multicellular bodies, wherein a plurality of multicellular bodies can be fused to form a planar layer. In some embodiments, a cell can be grown three dimensionally. In some embodiments, a cell can be grown in suspension.

In some embodiments, forming a cell layer can comprise using a scaffold. In some embodiments, a cell layer can be formed by arranging a plurality of multicellular bodies on a scaffold. In some embodiments, a forming step can comprise arranging or placing multicellular bodies on a support substrate that allows a multicellular body to fuse to form a layer (e.g., a substantially planar layer). In some embodiments, a multicellular body or a layer can be arranged horizontally and/or vertically adjacent to one another. In some embodiments, forming a cell layer may not need a scaffold.

In some embodiments, a cell layer can be formed by embedding cells in a medium or gel. In some embodiments, a dermal layer can be formed using a fibroblast embedded in a collagen I or a fibrin gel. In some embodiments, other types of medium can also be used. In some embodiments, a medium can promote a fibroblast to secrete sufficient amount of extracellular matrix to enable extended maintenance of epidermis without a need for collagen gels.

In some embodiments, there can be various ways to make multicellular bodies having characteristics described herein, for example comprising one or more engineered cells disclosed herein. In some embodiments, a multicellular body can be fabricated from a cell paste containing a plurality of cells, e.g., with a desired cell density and viscosity. In some embodiments, a multicellular body can comprise an engineered cell. In further cases, a cell paste can be shaped into a desired shape and a multicellular body formed through maturation (e.g., incubation). In some embodiments, an elongate multicellular body can be produced by shaping a cell paste including a plurality of cells into an elongate shape (e.g., a cylinder). In further cases, a cell paste can be incubated in a controlled environment to allow cells to adhere and/or cohere to one another to form an elongate multicellular body. In some embodiments, a multicellular body can be produced by shaping a cell paste including a plurality of living cells in a device that holds a cell paste in a three-dimensional shape. In some embodiments, a cell paste can be incubated in a controlled environment while it may be held in a three-dimensional shape for a sufficient time to produce a body that has sufficient cohesion to support itself on a flat surface, as described herein.

In some embodiments, a cell paste can be provided by: (A) mixing cells or cell aggregates (of one or more cell types) and a cell culture medium (e.g., in a pre-determined ratio) to result in a cell suspension, and (B) compacting a cellular suspension to produce a cell paste with a desired cell density and viscosity. Compacting can be achieved by a number of methods, such as by concentrating a particular cell suspension that resulted from cell culture to achieve a desired cell concentration (density), viscosity, and consistency required for a cell paste. In some embodiments, a relatively dilute cell suspension from cell culture can be centrifuged for a determined time to achieve a cell concentration in a pellet that allows shaping in a mold. Tangential flow filtration (“TFF”) can be another suitable method of concentrating or compacting cells. In some embodiments, compounds can be combined with a cell suspension to lend a required extrusion property. Suitable compounds include, collagen, hydrogels, Matrigel, nanofibers, self-assembling nanofibers, gelatin, and fibrinogen. One or more ECM components (or derivatives of ECM components) can also be included by, resuspending a cell pellet in one or more physiologically acceptable buffers containing an ECM components (or derivatives of ECM components) and a resulting cell suspension centrifuged again to form a cell paste.

In some embodiments, various methods can be used to shape a cell paste. For example, in a particular embodiment, a cell paste can be manually molded or pressed (e.g., after concentration/compaction) to achieve a desired shape. By way of a further example, a cell paste can be taken up (e.g., aspirated) into a preformed instrument, such as a micropipette (e.g., a capillary pipette), that shapes a cell paste to conform to an interior surface of an instrument. In some embodiments, a cross-sectional shape of a micropipette (e.g., capillary pipette) can be alternatively circular, square, rectangular, triangular, or other non-circular cross-sectional shape. In some embodiments, a cell paste can be shaped by depositing it into a preformed mold, such as a plastic mold, metal mold, or a gel mold. In some embodiments, centrifugal casting or continuous casting can be used to shape a cell paste.

In some embodiments, a cell paste can be further matured. In some embodiments, a cell paste can be incubated at about 37° C. for a time period (which can be cell-type dependent) to foster adherence and/or coherence. Alternatively, or in addition, a cell paste can be held in a presence of cell culture medium containing factors and/or ions to foster adherence and/or coherence.

Multicellular bodies can be arranged on a support substrate to produce a desired three-dimensional structure (e.g., a substantially planar layer). For example, multicellular bodies can be manually placed in contact with one another, deposited in place by extrusion from a pipette, nozzle, or needle, or positioned in contact by an automated machine such as a biofabricator.

A support substrate can be permeable to fluids, gasses, and nutrients and allows cell culture medium to contact all surfaces of a multicellular bodies and/or layers during arrangement and subsequent fusion. In some embodiments, a support substrate can be made from natural biomaterials such as collagen, fibronectin, laminin, and other extracellular matrices. In some embodiments, a support substrate can be made from synthetic biomaterials such as hydroxyapatite, alginate, agarose, polyglycolic acid, polylactic acid, and their copolymers. In some embodiments, a support substrate can be solid, semisolid, or a combination of solid and semisolid support elements. In some embodiments, a support substrate can be planar to facilitate production of planar layers. In some embodiments, a support substrate can be raised or elevated above a non-permeable surface, such as a portion of a cell culture environment (e.g., a Petri dish, a cell culture flask, etc.) or a bioreactor. A permeable, elevated support substrate can contribute to prevention of premature cell death, contributes to enhancement of cell growth, and facilitates fusion of multicellular bodies to form layers.

Once assembly of a layer may be complete, a tissue culture medium can be poured over a top of a construct. In some embodiments, a tissue culture medium can enter a space between multicellular bodies to support cells in the multicellular bodies. In some embodiments, multicellular bodies in a three-dimensional construct can be allowed to fuse to one another to produce a layer (e.g., a substantially planar) for use in formation of a synthetic leather. By “fuse,” “fused” or “fusion,” it can be meant that cells of contiguous multicellular bodies become adhered and/or cohered to one another, either directly through interactions between cell surface proteins, or indirectly through interactions of cells with ECM components or derivatives of ECM components. In some embodiments, a fused layer can be completely fused and multicellular bodies can become substantially contiguous. Alternatively, a fused layer can be substantially fused or partially fused and the cells of the multicellular bodies have become adhered and/or cohered to an extent necessary to allow moving and manipulating the layer intact.

Multicellular bodies can fuse to form a layer in a cell culture environment (e.g., a Petri dish, cell culture flask, or bioreactor). In some embodiments, the multicellular bodies fuse to form a layer in an environment with conditions suitable to facilitate growth of the cell types included in the multicellular bodies. In some embodiments, fusing takes place over about 15, 20, 25, 30, 35, 40, 45, 50, 55, and 60 minutes, and increments therein. In other cases, fusing takes place over about 1, 2, 3, 4, 5, 6, 7, 8, 9, 10, 11, 12, 14, 16, 18, 20, 22, 24, 26, 28, 30, 32, 34, 36, 38, 40, 42, 44, 46, and 48 hours, and increments therein. In yet other cases, fusing takes place over about 1, 2, 3, 4, 5, 6, 7, 8, 9, 10, 12, and 14 days, and increments therein. In further cases, fusing takes place over about 2 hours to about 24 hours. Factors relevant to the fusing time can include cell types, cell type ratios, culture conditions, and the presence of additives such as growth factors.

Once fusion of a layer may be complete, the layer and the support substrate can be separated. In some embodiments, the layer and the support substrate can be separated when fusion of a layer can be substantially complete or partially complete, but the cells of the layer can be adhered, cohered or any combination thereof to one another to an extent necessary to allow moving, manipulating, and stacking the layer without breaking it apart. The layer and the support substrate can be separated via standard procedures for melting, dissolving, or degrading the support substrate. In some embodiments, the support substrate can be dissolved, for example, by temperature change, light, or other stimuli that do not adversely affect the layer. In certain cases, the support substrate can be made of a flexible material and peeled away from the layer. The separated layer can be transferred to a bioreactor for further maturation. In some embodiments, the separated layer matures and further fuses after incorporation into an engineered animal skin, hide, or leather product.

Alternatively, the layer and the support substrate may not be separated. The support substrate degrades or biodegrades prior to packaging, freezing, sale or consumption of an assembled engineered animal skin, hide, or leather product.

A cell layer can be formed over a period of time. In some embodiments, a cell layer, e.g., an epidermal layer or a dermal layer, can be formed within 1, 2, 3, 4, 5, 6, 7, 8, 9, 10, 11, 12, 13, 14, 15, 16, 17, 18, 19, 20, 25, 30, 40, 50, 60, 120, 300 days. In some embodiments, a dermal layer can be formed in about 1 to 15 days, e.g., 5 to 10 days, or 10 to 12 days. In some embodiments, a dermal layer can be formed about 5 to 25 days, e.g., 14 to 15 days.

The present disclosure provides methods for making synthetic leather improved barrier function. In some embodiments, the methods can comprise providing keratinocytes and a culture medium comprising ascorbic acid and linoleic acid; and culturing the keratinocytes under conditions such that a synthetic leather having improved barrier function can be formed. In some embodiments, the culture conditions include culture at about 50 to 95% humidity, e.g., about 75% humidity. In some embodiments, an ascorbic acid can be provided at concentration of from about 10 to 100 micrograms/ml. In still further cases, linoleic acid can be provided at a concentration of from about 5 to 80 micromolar. The present disclosure may not be limited to synthetic leather formed from a particular source of keratinocytes. Indeed, the synthetic leather can be formed from a variety of primary and immortal keratinocytes, including, but not limited to Near-Diploid Immortalized Keratinocytes (NIKS) cells. In some embodiments, the synthetic leather can be formed by an engineered cell comprising a molecular switch. In some embodiments, an engineered cell can be an immortal cell (e.g. an immortalized bovine fibroblast). In still further cases, the keratinocytes express exogenous wild-type or variant Kruppel-like factor (GKLF). In still further cases, the keratinocytes can be derived from two different sources. In other cases, the synthetic leather has a surface electrical capacitance of from about 40 to about 240 pF. In some preferred cases, the skin equivalent has a surface electrical capacitance of from about 80 to about 120 pF. In other preferred cases, the content of ceramides 5, 6, and 7 in the skin equivalent can be from about 20 to about 50% of total ceramide content. In still other preferred cases, the content of ceramide 2 in the skin equivalent can be from about 10 to about 40% of total ceramide content. In still further cases, the present disclosure provides the skin equivalent made by the method just described.

Multiple cell layers can be arranged to form a layer structure, thus producing synthetic leathers described herein. In some embodiments, dermal layers and epidermal layers can be formed separately and assembled by placing epidermal layers atop of the dermal layers (e.g., when both an epidermal layer and a dermal layer can be fully formed). In some embodiments, an epidermal layer can be grown atop a dermal layer. In certain cases, a basement membrane or basement membrane substitute can be placed between a dermal layer and an epidermal layer. For example, the cell layers can be manually placed in contact with one another or deposited in place by an automated, computer-aided machine such as a biofabricator, according to a computer script.

Before assembling multiple cell layers, one or more quality control steps can be performed. For example, Trans Epithelial Electrical Resistance (TEER) can be performed on epidermis before placement on dermis (e.g., 0 day), followed by histology analysis (e.g., minimum 3-5 days). Using methods provided herein, the risk of improperly formed layered structure or full thickness skin equivalents can be low.

Multiple cell layers can be assembled in various ways. In some embodiments, an epidermal layer and a dermal layer (with or without a basement membrane substitute) can be placed on a scaffold (e.g., silk), e.g., to achieve thickness and tensile strength of natural leather. In some embodiments, an epidermal layer and multiple dermal layers (with or without a basement membrane substitute) can be assembled without using a scaffold. Such assembly can achieve thickness and tensile strength that resemble natural leather. In some embodiments, an epidermal layer and multiple dermal layers (with or without a basement membrane substitute) can be placed on a scaffold (e.g., silk) achieve thickness and tensile strength that resemble natural leather. In some embodiments, the leather has a tensile strength of at least about: 10, 20, 40, 50, 60, 70, 80, 90, 100, 125, 150, 175, 200, 250, or 300 kgf/cm². In some embodiments, the leather has a tensile strength of less than about: 50, 60, 70, 80, 90, 100, 125, 150, 175, 200, 250, or 300 kgf/cm². In certain embodiments, tensile strength testing can be carried out according to American Society for Testing and Materials (ASTM) standards.

Multiple cell layers can be assembled to form a synthetic leather (e.g., a full thickness skin equivalent). A synthetic leather can comprise a top part, a middle part and a bottom part. The top part can comprise an epidermal layer. For example, the top part can be a single layer epidermal layer. The middle part can comprise a basement membrane substitute. In some embodiments, the middle part does not have a basement membrane substitute. For example, the middle part can have a layer of negligible thickness. The bottom part can have one or more dermal layers. In some embodiments, the bottom part has a single dermal layer placed on a scaffold (e.g., silk). In some embodiments, the bottom part has multiple dermal layers (e.g., up to 5 layers) without any scaffold. In some embodiments, the bottom part has multiple dermal layers stacked atop each other and placed on a scaffold (e.g., silk).

Adhesiveness between epidermal and dermal layers can be strong enough to resist layer splitting. In some embodiments, the cells layers can be assembled by adhering on to a scaffold. Natural or synthetic adhesives can be used for an assembly. A natural adhesive can be fibrin glue, cold glues, animal glue (e.g., bone glue, fish glue, hide glue, hoof glue, rabbit skin glue, meat glue), blood albumen glue, casein glue, vegetable glues (e.g., starch, dextrin glues, Canada balsam, pine rosin based glue, cocconia, gum Arabic, postage stamp gum, latex, library paste, methyl cellulose, mucilage, resorcinol resin, or urea-formaldehyde resin), or any combination thereof. A synthetic adhesive can be Acrylonitrile, Cyanoacrylate (e.g., n-buthyl-2-cyanoacrylate glue), Acrylic, Resorcinol glue, Epoxy resins, Epoxy putty, Ethylene-vinyl acetate, Phenol formaldehyde resin, Polyamide, Polyester resins, Polyethylene, Polypropylene, Polysulfides, Polyurethane, Polyvinyl acetate (including white glue (e.g. Elmer's Glue) and yellow carpenter's glue (Aliphatic resin), Polyvinyl alcohol, Polyvinyl chloride (PVC), Polyvinyl chloride emulsion (PVCE), Polyvinylpyrrolidone Rubber cement, Silicones, and Styrene acrylic copolymer. For example, an assembly can be performed using fibrin glue. For example, an assembly can be performed using n-buthyl-2-cyanoacrylate glue.

In some embodiments, cell layers (e.g., substantially planar layers) can be stacked to form a synthetic leather. A cell layer can have an orientation defined by the placement, pattern, or orientation of multicellular bodies. In some embodiments, each layer can be stacked with a particular orientation relative to the support substrate and/or one or more other layers. For example, one or more layers can be stacked with an orientation that includes rotation relative to the support substrate and/or the layer below, wherein the rotation can be between 0.1 and 180 degrees, for example, about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, and 180 degrees, or increments therein. In other cases, all layers can be oriented substantially similarly.

Once stacking of the layers may be complete, the layers in the three-dimensional construct can be allowed to fuse to one another to produce a synthetic leather. In some embodiments, the layers fuse in a cell culture environment (e.g., a Petri dish, cell culture flask, bioreactor, etc.). In some embodiments, the fusing take place over about 15, 20, 25, 30, 35, 40, 45, 50, 55, and 60 minutes, and increments therein. In other cases, fusing takes place over between 1 and 48 hours, e.g., over about 1, 2, 3, 4, 5, 6, 7, 8, 9, 10, 11, 12, 14, 16, 18, 20, 22, 24, 26, 28, 30, 32, 34, 36, 38, 40, 42, 44, 46, and 48 hours, and increments therein. For example, fusing can take place over about 2 hours to about 24 hours.

In some embodiments, a cell or cell layer can be cultured in various cell culture conditions. In some embodiments, the cell and cell layers can be incubated in a medium, a bioreactor, an incubator or any combination thereof. In some embodiments, a medium can comprise media. In some embodiments, a cell or cell layer can be cultured in vitro. For example, a dermal layer and/or an epidermal layer can be cultured in vitro. Alternatively, the cells or cell layers can be cultured in vivo. For example, a dermal layer and/or an epidermal layer can be cultured in vivo. In some embodiments, a medium can comprise a culture medium as disclosed herein. In some embodiments a culture medium can comprise a growth medium, a tissue formation medium, or a combination thereof, as disclosed herein. In some embodiments, a cell, a cell layer, a portion thereof, or any combination thereof can be cultured in a medium. In some embodiments, a medium can comprise a supplement. In some embodiments, a supplement can be a natural supplement, a synthetic supplement, or a combination thereof. In some embodiments, a supplement can be an additive. In some embodiments, one or more of the supplements can induce production and assembly of an extracellular matrix from engineered cells, thus enhancing a natural look of a synthetic leather. In some embodiments, a supplement can comprise an ECM component such as a collagen, a fibrin, a growth factor, a small molecules, a macromolecule, a sulphate, a carrageenan, or any combination thereof. In some embodiments, a small molecule can comprise ascorbic acid. In some embodiments, a macromolecule can comprise a dextran. In some embodiments, a cell can be grown in a cell culture medium. In some embodiments, a cell culture medium can comprise a growth medium, a tissue formation medium, or a combination thereof. In some embodiments, a culture medium can comprise Dulbecco's modified eagle's medium (DMEM), RPMI-1640, Eagle's Minimal Essential Medium, Ham's nutrient mixtures, Iscove's modified medium, Dulbecco's medium, or any combination thereof. In some embodiments, a supplement can be added to a culture medium such as a salt, a buffering reagent, a phenol red, a HEPES, an amino acid, a carbohydrate, a lipid, a protein, a peptide, a fatty acid, a vitamin, an element, a medium supplement, an antibiotic, a serum, a serum alternative or any combination thereof. In some embodiments, a medium can comprise about 0%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, or about 100% of a serum, a serum alternative, or a combination thereof. In some embodiments, a medium can comprise from about 0.10%, to about 10%, from about 10%, to about 20%, from about 30%, to about 40%, from about 40%, to about 50%, from about 50%, to about 60%, from about 60%, to about 70%, from about 70%, to about 80%, from about 80%, to about 90%, from about 90%, to about 100% of a serum, a serum alternative, or a combination thereof.

In some embodiments, a medium can comprise a growth medium, a tissue formation medium, or a combination thereof. In some embodiments, after a seeding, a transfected or transduced isolated cell can be contacted with a tissue formation medium. In some embodiments, a growth medium can comprise a growth factor, a buffer, a salt, a sugar, an amino acid, a lipid, a mineral, an ECM protein, or a combination thereof. In some embodiments, a salt can comprise an inorganic salt. In some embodiments, an inorganic salt can comprise a Calcium Chloride, a Ferric Nitrate, a Magnesium Sulfate (anhydrous), a Potassium Chloride, a Sodium Bicarbonate, a Sodium Chloride, a Sodium Phosphate Monobasic (anhydrous), or any combination thereof. In some embodiments, an amino acid can comprise an L-Arginine·HCl, an L-Cystine·2HCl, a Glycine, an L-Histidine·HCl·H2O, an L-Isoleucine, an L-Leucine, an L-Lysine·HCl, an L-Methionine, an L-Phenylalanine, an L-Serine, an L-Threonine, an L-Tryptophan, an L-Tyrosine·2Na·2H2O, an L-Valine, an L-Glutamine, a stereoisomer of any of these, a salt of any of these, or any combination thereof. In some embodiments, a vitamin can comprise a Choline Chloride, a Folic Acid, a myo-Inositol, a Niacinamide, a D-Pantothenic Acid (hemicalcium), a Pyridoxal·HCl, a Pyridoxine·HCl, a Riboflavin, a Thiamine·HCl, a stereoisomer of any of these, a salt of any of these, or any combination thereof. In some embodiments, a sugar can comprise D-Glucose, a stereoisomer thereof, a salt thereof, or any combination thereof. In some embodiments, a pH indicator can comprise a Phenol Red·Na, a Pyruvic Acid·Na, a stereoisomer of any of these, a salt of any of these, or any combination thereof. In some embodiments, a growth medium can comprise an amino acid, a vitamin, an inorganic salt, a fetal bovine serum, an antibiotic, an antimycotic, or any combination thereof. In some embodiments, a tissue formation medium can comprise a growth factor, a buffer, a salt, a sugar, an amino acid, a lipid, a mineral, an ECM protein, a human platelet lysate, an acid citrate dextrose, a heparin, an ascorbic acid, a TGF-β1, a normocin, a serum, a serum alternative, a non-essential amino acid, an antibiotic, an antimycotic, or any combination thereof. In some embodiments, a tissue formation medium further can comprise a serum, a serum alternative, or a combination thereof. In some embodiments, an amino acid can comprise a Glycine, an Alanine, an L-Arginine hydrochloride, an L-Asparagine-H2O, an L-Aspartic acid, an L-Cysteine hydrochloride-H2O, an L-Cystine 2HCl, an L-Glutamic Acid, an L-Glutamine, an L-Histidine hydrochloride-H2O, an L-Isoleucine, an L-Leucine, an L-Lysine hydrochloride, an L-Methionine, an L-Phenylalanine, an L-Proline, an L-Serine, an L-Threonine, an L-Tryptophan, an L-Tyrosine disodium salt dihydrate, an L-Valine, a stereoisomer of any of these, a salt of any of these, or any combination thereof. In some embodiments, a vitamin can comprise a biotin, a choline chloride, a D-Calcium pantothenate, a Folic acid, a Niacinamide, a Pyridoxine hydrochloride, a Riboflavin, a Thiamine hydrochloride, a Vitamin B12, an i-Inositol, a stereoisomer of any of these, a salt of any of these, or any combination thereof. In some embodiments, an inorganic salt can comprise a calcium chloride, a cupric sulfate, a ferric nitrate, a ferric sulfate, a magnesium chloride, a magnesium sulfate, a potassium chloride, a sodium bicarbonate, a sodium chloride, a sodium phosphate dibasic, a sodium phosphate monobasic, a zinc sulfate, a stereoisomer of any of these, a salt of any of these, or any combination thereof. In some embodiments, a medium further can comprise a D-Glucose (Dextrose), a hypoxanthine Na, a linoleic acid, a lipoic acid, a phenol red, a putrescine 2HCl, a Sodium pyruvate, a thymidine, a stereoisomer of any of these, a salt of any of these, or any combination thereof. In some embodiments, a serum alternative can contain substantially no animal derived products, can be xeno-free, or a combination thereof. In some embodiments, a serum alternative can comprise a growth factor, an insulin transferrin, a cytokine, an essential amino acid, a nonessential amino acid, a protein, an extracellular matrix protein (ECM), a laminin, a fibronectin, a vitronectin, a cell adhesion peptide, an RGD, an extracellular matrix fragment, a hormone, a collagen, an albumin, a lipid, a glycoprotein, a protein fragment, or any combination thereof. In some embodiments, a serum can comprise a fetal bovine serum (FBS), a horse serum, a fetal calf serum, or any combination thereof. In some embodiments, a medium does not comprise TGF beta. In some embodiments, after a seeding, a transfected or transduced isolated cell can be contacted with a tissue formation medium. In some embodiments, contacting a cell with a tissue formation medium can at least partially cause a cell to differentiate or form into another cell or tissue type. In some embodiments, a transfected or transduced isolated cell (a) at least partially increases production of a collagen, (b) has an at least partially arrested cell growth, or (c) both, when present in a tissue formation medium, relative to an otherwise comparable transfected or transduced isolated cell that has not been contacted with a tissue formation medium. In some embodiments, a transfected or transduced isolated cell can be at least partially differentiated when contacted with a tissue formation medium. In some embodiments, after a seeding, a transfected or transduced isolated cell can be contacted with a medium comprising L-ascorbic acid 2-phosphate (AA2P), a salt thereof, a biologically active fragment thereof, or a combination of any of these. In some embodiments, a transfected or transduced isolated cell (a) at least partially increases production of collagen, (b) has an at least partially arrested cell growth, or (c) both, when present in a medium comprising an AA2P, a salt thereof, a TGFB1, a biologically active fragment thereof, or a combination, relative to an otherwise comparable medium lacking an AA2P, a salt thereof, a TGFB1, a biologically active fragment thereof, or a combination thereof. In some embodiments, a transfected or transduced isolated cell can be at least partially differentiated when contacted with a medium.

In some embodiments, a stimulus can comprise a presence, absence or level of a carbohydrate, a lipid, a nucleic acid, a protein, an antibiotic, an organic chemical, an inorganic chemical, an artificial chemical, a metabolite, or any combination thereof. In some embodiments, a cell, a cell layer, or a combination thereof, can be cultured with one or more stimuli. In some embodiments, a stimulus can be added before proliferation, during proliferation, after proliferation or any combination thereof.

In some embodiments, growing a single cell can be performed in a shaker, a spinner flask, or any combination thereof. In some embodiments, a growing of a tissue can comprise growing with or without shaking. In some embodiments, a growing of a tissue can comprise growing first with shaking at a high speed followed by little or no shaking. In some embodiments, a growing of a tissue can comprise growing first with little or no shaking followed by shaking at a high speed. In some embodiments, little or no shaking can be at most 80, 70, 60, 50, 40, 35, 30, 25, 20, 15, 10, 5, 3, 2, 1 or 0 revolutions per minute (RPM). In some embodiments, little or no shaking can be for more than about: 6 hours, 12 hours, 18 hours, 24 hours, 48 hours, 72 hours, 4 days, 5 days, 6 days, 1 week, 2, weeks, 3 weeks, or 4 weeks. In some embodiments, little or no shaking can be for less than about: 6 hours, 12 hours, 18 hours, 24 hours, 48 hours, 72 hours, 4 days, 5, days, 6 days, 1 week, 2 weeks, 3 weeks or 4 weeks. In some embodiments, the little of no shaking can be for more than about: 1, 2, 3, 4, 5, 6, 7, 8, 9, 10, 15, or 20 passages. In some embodiments, the little of no shaking can be for less than about: 1, 2, 3, 4, 5, 6, 7, 8, 9, 10, 15, or 20 passages. In some embodiments, high speed shaking can be at least 60, 80, 100, 120, 140, 160, 180, 200, 220 or 240 RPM. In some embodiments, high speed shaking can be between 180-240, 200-240, 160-220, 60-160, 60-140, 60-120, 60-100, 60-90, 60-80, 80-160, 80-140, 80-120, 80-100, or 80-90 RPM. In some embodiments, high speed shaking can be for more than about: 2, 3, 4, 5, 7, 10, 15 or 20 passages. In some embodiments, high speed shaking can be for less than about: 2, 3, 4, 5, 7, 10, 15 or 20 passages. In some embodiments, shaking can performed at an initial speed and then increased to a higher speed. In some embodiments, shaking can be performed at an initial speed and then decreased to a lower speed. In some embodiments, shaking can be at a consistent speed.

In some embodiments, proliferation of an engineered cell can be determined by manual cell counting, automated cell counting, indirect cell counting or any combination thereof. In some embodiments, cell counting can comprise a counting chamber, colony forming unit counting, electrical resistance, flow cytometry, image analysis, stereologic cell, spectrophotometry, impedance microbiology, automated cell counting, microscopy, Coulter counter, automated image cell counter, droplet digital polymerase chain reaction (PCR), quantitative PCR, metabolic measurements or any combination thereof.

In some embodiments, an anchorage independent cells may be capable of anchorage independent growth for at least 1, 2, 3, 4, 5, 7, 10, 12, 15, 17, 20, 22, 25, 27, 30, 32, 34, 35, 37 or 40 cellular divisions. an anchorage-independent cells may be capable of anchorage dependent growth for at least 1, 2, 3, 4, 5, 7, 10, 12, 15, 17, 20, 22, 25, 27, 30, 32, 34, 35, 37 or 40 cellular divisions. A cellular division can be also referred to herein as a passage. In some embodiments, an anchorage independent cells can be capable of anchorage independent growth indefinitely. In some embodiments, an anchorage dependent cells can be capable of anchorage dependent growth indefinitely. In some embodiments, an anchorage-independent cells may be capable of anchorage independent growth for at least 1 passage, 5 passages or for at least 35 passages. In some embodiments, an anchorage dependent cells may be capable of anchorage dependent growth for at least 1 passage, 5 passages or for at least 35 passages. In some embodiments, a cell can be programed after a set amount of divisions to switch from anchorage independent proliferation to anchorage dependent proliferation. In some embodiments, a cell can be programed after a set amount of divisions to switch from anchorage dependent proliferation to anchorage independent proliferation. In some embodiments, a stimulus can control the programed switch. In some embodiments, a stimulus may not control the programed switch. For example, a cell in anchorage independent proliferation can go through about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 rounds of division and switch to anchorage dependent proliferation. In some embodiments, a cell in anchorage dependent proliferation can go through about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 rounds of division and switch to anchorage independent proliferation. In some embodiments, an anchorage independent cell can be at least partially incapable of adherent growth. In some embodiments, an anchorage dependent cell can be at least partially incapable of growth in suspension. In some embodiments, at least 30%, 40%, 50%, 55%, 60%, 65%, 70%, 75%, 80%, 85%, 90%, 95%, 97%, 99% or 100% of an anchorage-independent cells may be incapable of adherent growth. In some embodiments, at least 30%, 40%, 50%, 55%, 60%, 65%, 70%, 75%, 80%, 85%, 90%, 95%, 97%, 99% or 100% of an anchorage dependent cell may be incapable of growth in suspension.

In some embodiments, cells can be seeded onto a scaffold. In some embodiments, cells may not be seeded on a scaffold. In some embodiments, cells can be seeded at a density from about: 100 cells/cm² to about 10,000,000 cells/cm², 1,000 cells/cm² to about 1,000,000 cells/cm², 100,000 cells/cm² to about 10,000,000 cells/cm², 10,000 cells/cm² to about 100,000 cells/cm², 50,000 cells/cm² to about 200,000 cells/cm², 100,000 cells/cm² to about 1,000,000 cells/cm², 100,000 cells/cm² to about 500,000 cells/cm² or about 100,000 cells/cm² to about 2,500,000 cells/cm². In some embodiments, cells can be seeded at a density of more than about: 10, 100, 10,000, 50,000, 75,000, 90,000, 100,000, 125,000, 150,000, 175,000, 200,000, 1,000,000 or 10,000,000 cells/cm². In some embodiments, cells can be seeded at a density of less than about: 10, 100, 10,000, 50,000, 75,000, 90,000, 100,000, 125,000, 150,000, 175,000, 200,000, 1,000,000 or 10,000,000 cells/cm². In some embodiments, the cells can be seeded on one side of a scaffold. In some embodiments, the scaffold can be a substrate. In some embodiments, the cells can be seeded on more than one side of a scaffold, for example seeding the top and the bottom of a scaffold. A scaffold can be seeded consecutively of concurrently. In some embodiments, a scaffold can be seeded on more than one side of a scaffold without flipping the scaffold. For example, a second side of the scaffold can be seeded without flipping the scaffold. In some embodiments, a scaffold can be seeded on one side then the scaffold may be flipped and another side of the scaffold may be seeded. In some embodiments, seeding cells can comprise a biofabricated material. In some embodiments, a biofabricated material can comprise a cell, an engineered cell, or a tissue disclosed herein.

In some embodiments, the cells and cell layers described herein can be cultured in anchorage independent conditions, anchorage dependent conditions, or any combination thereof. In some embodiments, anchorage independent proliferation, anchorage dependent proliferation, or both can occur in a vessel, a container or a combination thereof. In some embodiments, anchorage independent or anchorage dependent proliferation can be measured against cells that may have not received a stimulus, may not contain a switch, or any combination thereof. In some embodiments, proliferation of anchorage dependent or proliferation of anchorage independent can be determined by manual cell counting, automated cell counting, indirect cell counting or any combination thereof. In some embodiments, at least partially anchorage dependent proliferation can consume more, same or less nutrients as compared to at least partially anchorage independent proliferation. In some embodiments, a vessel or a container can be glass, metal, plastic, or any combination thereof. In some embodiments, the container or vessel can contain a coating that causes the cells to adhere to at least a portion of the vessel or container. In some embodiments, a coating can be poly-L-lysine, poly-D-lysine, laminin, collagen, bovine fibronectin, gelatin, extracellular matrix, laminin, entactin, HSPG, bovine gelatin, or any combination thereof. In some embodiments, the vessel or a container can hold a total volume of at least about: 0.5 mL, 1 mL, 5 mL, 25 mL, 100 mL, 250 mL, 500 mL, 1000 mL, 2 L, 5 L, 10 L, 100 L, 1000 L, 5000 L or about 10,000 L. In some embodiments, in anchorage independent proliferation conditions, cells can be grown to about: 1e¹ cells/mL, 1e² cells/mL, 1e³ cells/mL, 1e⁴ cells/mL, 1e⁵ cells/mL, 1e⁶ cells/mL, 1e⁷ cells/mL, 1e⁸ cell/mL, 1e⁹ cells/mL or 1e¹⁰ cells/mL In some embodiments, cells in anchorage independent proliferation can be grown to a confluency of less than about: 1%, 2%, 3%, 4%, 5%, 10%, 20%, 30%, 40%, 50%, 60%, 70%, 80%, or 90%. In some embodiments, cells in anchorage independent proliferation can be grown to a confluency of more than about: 1%, 2%, 3%, 4%, 5%, 10%, 20%, 30%, 40%, 50%, 60%, 70%, 80%, or 90%. In some embodiments, cells in anchorage independent proliferation can be grown in a suspension. In anchorage dependent proliferation conditions, cells can be grown to about: 1e¹ cells/mL, 1e² cells/mL, 1e³ cells/mL, 1e⁴ cells/mL, 1e⁵ cells/mL, 1e⁶ cells/mL, 1e⁷ cells/mL, 1e⁸ cell/mL, 1e⁹ cells/mL or 1e¹⁰ cells/mL. In some embodiments, when cells in anchorage independent proliferation reach a density of about: 1e⁵ cells/mL, 1e⁶ cells/mL, 1e⁷ cells/mL, 1e⁸ cell/mL, 1e⁹ cells/mL or 1e¹⁰ cells/mL cells can switch to anchorage dependent proliferation. In some embodiments, when cells in anchorage dependent proliferation reach a density of about: 1e⁵ cells/mL, 1e⁶ cells/mL, 1e⁷ cells/mL, 1e⁸ cell/mL, 1e⁹ cells/mL or 1e¹⁰ cells/mL cells can switch to anchorage dependent proliferation. In anchorage dependent proliferation conditions cells can be grown to a density of substrate surface area from about: 50,000 cells/cm² to about 9,000,000 cells/cm², 200,000 cells/cm² to about 4,500,000 cells/cm², 250,000 cells/cm² to about 4,000,000 cells/cm², 500,000 cells/cm² to about 2,000,000 cells/cm², or 1,000,000 cells/cm² to about 6,000,000 cells/cm². In some embodiments, cells in anchorage dependent proliferation can be grown to a confluency of more than about: 5%, 10%, 20%, 30%, 40%, 50%, 60%, 70%, 80%, 90%, 95%, or 99%. In some embodiments, cells in anchorage dependent proliferation can be grown to a confluency of less than about: 5%, 10%, 20%, 30%, 40%, 50%, 60%, 70%, 80%, 90%, 95%, or 99%. Cells in anchorage dependent proliferation conditions can be grown at least partially on, in or around a scaffold. In some embodiments, cells in anchorage dependent proliferation can be grown at least partially without a scaffold.

In some embodiments, cells disclosed herein and structures can be cultured with certain air humidity. For example, the cell layers (e.g., engineered cells, dermal layers or epidermal layers) can be cultured at from about 20% to about 100% humidity. For example, the humidity can be from about 40% to about 100%, from about 50% to about 95%, from about 45% to about 90%, from about 55% to about 95%, from about 60% to about 90%, from about 70% to about 80%, from about 71% to about 79%, from about 72% to about 78%, from about 73% to about 77%, from about 74% to about 76%, from about 60% to about 70%, from about 65% to about 75%, from about 70% to about 80%, from about 75% to about 85%, from about 80% to about 90%, from about 85% to about 95%, or from about 90% to about 100%, from about 40% to about 60%, from about 45% to about 55%, from about 46% to about 54%, from about 47% to about 53%, from about 48% to about 52%, from about 48% to about 53%, from about 49% to about 54%, or from about 47% to about 51%.

In some embodiments, cells and tissues (e.g. an immortalized cell with a switch) can be grown in a bioreactor or a device that supports a biologically active environment. A device or bioreactor can comprise a cell stack, a roller bottle, a shaker, flasks, a stirred tank suspension bioreactor, a high cell density fixed bed perfusion bioreactor, incubator, or any combination thereof. In some embodiments, a bioreactor can be a scalable, modular system from the production of cell culture and can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 containers. In some embodiments, a bioreactor can be a scalable system, a stackable system, a modular system or any combination thereof. Further, in some embodiments, a bioreactor can be configured to allow easy, rapid scaling up. Furthermore, a bioreactor can include a motion control system configured to rock or tilt a container, which can create a dynamic flow within the chamber that can increase viability of cell culture within the container. In some embodiments, a bioreactor can include a container in which cells can be grown, a temperature regulation system, a gas inlet/outlet system configured to regulate a gas concentration within the container, an agitator configured to mix growth medium within a container, any number of inlet and outlet ports for fluid transport. In some embodiments, a cell culture can be suspended in a growth medium in the container of the bioreactor. In some embodiments, the bioreactor contains a computer system to input commands. In some embodiments, a user can program settings that can dictate a speed of agitation, a desired pH, a temperature, and or a dissolved oxygen level. In some embodiments, a system can adjust these parameters based on the parameters input by the user. In some embodiments, a user or this system can manually or automatically siphon/change medium or other components using, for example, a pump operatively connected to a port in a container via a tube or pipe. In some embodiments, a medium can continuously flow in and out of a container of a system. In some embodiments, the system can utilize static motion (i.e., the bioreactor typically remains stationary during the growth). In some embodiments, a system described herein can include a motion control system configured to rock, rotate, or tilt a container, which can create a dynamic flow within the chamber that can increase viability of cell culture within the container.

In some embodiments, methods herein can comprise tanning at least a portion of a synthetic leather, e.g., at least a portion of a tissue. In some embodiments, a tissue can comprise, a fibroblast, a keratinocyte, a corneocyte, a melanocyte, a Langerhans cell, a basal cell, an adipocyte, a smooth muscle cell, an epithelial cell, an engineered cell, an immortalized cell or any combination thereof. Any cell described herein can be an immortalized cell. In some embodiments, a tissue can comprise a layered structure. In some embodiments, a tissue can comprise a dermis layer. In some embodiments, a tissue can comprise an epidermis layer. Tanning can make a synthetic leather resemble a natural leather, which can be a durable and flexible material created by the tanning of animal rawhide and skin, often cattle hide. Tanning herein can refer to the process of treating the skins of animals to produce leather. Tanning can be performed various ways, including vegetable tanning (e.g., using tannin), chrome tanning (chromium salts including chromium sulfate), aldehyde tanning (using glutaraldehyde or oxazolidine compounds), syntans (synthetic tannins, using aromatic polymers), bacterial dyeing, and the like. In some embodiment, tanning can be metal-free. In some embodiments, tanning can be an environmentally friendly process.

In some embodiments, tanning can be performed to convert proteins in the hide/skin into a stable material that will not putrefy, while allowing the material to remain flexible. Chromium can be used as tanning material. In some cases tanning can comprise a chromium, an aluminum, a zirconium, a titanium, an iron, a sodium aluminum silicate, a formaldehyde, a glutaraldehyde, an oxazolidine, an isocyanate, a carbodiimide, a polycarbamoyl sulfate, a tetrakis hydroxyphosphonium sulfate, a sodium p-[(4,6-dichloro-1,3,5-triazin-2-yl)amino] benzenesulphonate, a pyrogallol, a catechol, a syntan or any combination thereof. In some embodiments, tanning can be performed on engineered cells in a scaffold. In some embodiments, a tissue that can be tanned can comprise fibers or a plurality of fibers (e.g. polyester fibers, synthetic fibers, natural fibers). The pH of the cell layer or layered structure can be adjusted (e.g., lowered; e.g. to pH about 2.8-3.2) to enhance the tanning; following tanning the pH can be raised (“basification” to a slightly higher level, e.g., pH about 3.8-4.2). In some embodiments a pH described herein can be at least 1. In some embodiments, a pH described herein can be 14 or less.

In some embodiments, tanning can be performed on cell layers, e.g., dermal layers, epidermal layers, engineered cells, immortalized cell layers, laminin, fibronectin, collagen or any combination thereof. In some embodiments, tanning can be performed on a tissue (e.g., a tissue from an engineered cell). Tanning can also be performed on layered structures, e.g., layered structures comprising at least a dermal layer. In certain cases, tanning can be also performed on a synthesized leather. For example, tanning can be performed after forming cell layers, e.g., dermal layers or epidermal layers. For example, tanning can be performed after forming layered structures. Tanning can also be performed on cells that comprise a switch. For example, tanning can be performed on a layer of immortalized cells that comprise a molecular switch for anchorage independent and anchorage dependent proliferation.

In some embodiments, tanning can be performed on cells or cell layers that have been dehydrated. Water content of a synthetic leather after dehydration may be no more than about: 5%, 10%, 15%, 20%, 30%, 35%, 40%, 50%, 60%, 70% or 80%. In some embodiments, dehydration can include decellularizing cells to prevent contraction. In some embodiments, decellularization can be done with ethanol, propyl alcohol, isopropyl alcohol, butanol, methane, ethane, methyl acetate, ethyl acetate, butyl acetate, ethylene carbonate, hydrochloric ether, dichloromethane, chloroethylene, DMSO, acetone, diethyl ether, organic solvents, or any combination thereof.

In some embodiments, tanning can be performed by modify an extracellular matrix (ECM) material. Tanning can be performed by modifying collagen in an ECM. The tanning can be performed using a tanning agent, e.g., chromium(III) sulfate ([Cr(H₂O)₆]2(SO4)₃). Chromium(III) sulfate can dissolve to give the hexaaquachromium(III) cation, [Cr(H₂O)₆]3+, which at higher pH undergoes processes called olation to give polychromium(III) compounds that can be active in tanning, being the cross-linking of the collagen subunits. Some ligands include the sulfate anion, the collagen's carboxyl groups, amine groups from the side chains of an amino acids, as well as masking agents. Masking agents can be carboxylic acids, such as acetic acid, used to suppress formation of polychromium(III) chains. Masking agents can allow the tanner to further increase the pH to increase collagen's reactivity without inhibiting the penetration of the chromium(III) complexes. Tanning can increase the spacing between protein chains in collagen (e.g., from 10 to 17 Å), consistent with cross-linking by polychromium species, of the sort arising from olation and oxolation. The chromium can be cross-linked to the collagen. Chromium-tanned leather can contain between about 4% and 5% of chromium. This efficiency can be characterized by its increased hydrothermal stability of the leather, and its resistance to shrinkage in heated water. Other tanning agents can be used to tan the layered body and modify the collagen.

In some embodiments, tanning can also be performed using other minerals. In some embodiments, tanning can be performed using agent based on alum, zirconium, titanium, iron salts, or a combination thereof. Tanning can be performed at least partially free of chromium.

In some embodiments, tanning can also be performed by vegetable tanning. In some embodiments, vegetable tanning can comprise immersing hides in plant-based extracts. In some embodiments, plant-based extracts can comprise extracts of chestnut bark, oak bark, hemlock bark, mangrove bark, wattle bark, myrobalans bark, quebracho bark, mimosa bark, olive leaves, oak bark or any combination thereof. In some embodiments, tanning can be performed by wet white tanning (e.g., tanning with aldehydes, polycarbamoylsulfate, or any combination thereof). In some embodiments, tanning can be performed with a mineral free tanning system comprising Granofin® Easy F90, and ProSpread™. In some embodiments, retanning can be performed with Densotan® A, Catalix® 150, Lipoderm® N, Lipoderm® LA, Basyntan® NL, Relugan® Soft Ap, Tanicor® VX, Basyntan® SW, Granofin® TAP, Melioderm® HF Brown, formic acid, Terogtan, Lipsol MSG, Catalix® 150, Brun dye, or any combination thereof.

In some embodiments, engineered cells, immortalized cells, Cell layers, layered structures, and synthetic leathers made herein can be further processed after tanning. In some embodiments, methods provided herein further comprise one or more leather processing steps (e.g., those used in traditional leather formation). Examples of processing steps include: preserving, soaking, liming, unhairing, fleshing, splitting, deliming, reliming, bating, degreasing, frizing, bleaching, colouring, pickling, depickling, tanning, re-tanning (e.g., if color may be lost during processing), thinning, retanning, lubricating, crusting, wetting, sammying, shaving, rechroming, neutralizing, dyeing, fatliquoring, filling, stripping, stuffing, whitening, fixating, setting, drying, conditioning, milling (e.g., dry milling), staking, buffing, finishing, oiling, brushing, padding, impregnating, spraying, roller coating, curtain coating, polishing, plating, embossing, ironing, glazing, and tumbling. In some embodiments, a lubricant can be a fat, a biological oil, a mineral oil, a synthetic oil, a cod oil, a sulfonated oil, a polymer, a resin, an organofunctional siloxane, or any combination thereof. In some embodiments, a lubricant can include a surfactant, an anionic surfactant, a cationic surfactant, a cationic polymeric surfactant, an anionic polymeric surfactant, an amphiphilic polymer, a fatty acid, a modified fatty acid, a nonionic hydrophilic polymer, a nonionic hydrophobic polymer, a poly acrylic acid, a poly methacrylic, an acrylic, a natural rubber, a synthetic rubber, a resin, an amphiphilic anionic polymer, an amphiphilic anionic copolymer, am amphiphilic cationic polymer, an amphiphilic cationic copolymer or combinations thereof. In some embodiments, emulsions or suspensions of lubricants can be in water, alcohol, ketones, and other solvents

In some embodiments, a synthetic leather can be shaped by, for example, controlling the number, size, and arrangement of the multicellular bodies and/or the layers used to construct an animal skin, hide, or leather. In other cases, an animal skin, hide, or leather can be shaped by, for example, cutting, pressing, molding, or stamping. The shape the synthetic leather can be made to resemble a traditional animal skin, hide, or leather product.

In some embodiments, methods herein can comprise removing a portion of a synthetic leather produced herein. In some embodiments, the method can comprise removing at least a portion of epidermal layer to form a removed product. For example, the removing can be shaving.

In some embodiments, methods herein can comprise pigmenting the synthetic leather. In some embodiments, pigmentation can be performed by introducing pigments producing cells (e.g., melanocytes) in the synthetic leather. In some embodiments, the synthetic leather can comprise functional live melanocytes. The melanocytes can have a similar location to that in the human skin. In some embodiments, melanin can be constitutively produced by melanocytes. In some embodiments, melanin can be transferred to keratinocytes. In some embodiments, melanocytes can be produced upon stimulation, e.g., UV radiation or by propigmenting active agents, such as alpha melanocyte stimulating hormone (aMSH), endothelin 1 (ET1), stem cell factor (SCF), prostaglandins E2 and F2a (PGE2, PGF2a), basic fibroblast growth factor (bFGF) or nerve growth factor (NGF).

In some embodiments, cells in different layers of a synthetic leather, such as keratinocytes, melanocytes, or fibroblasts can be derived, e.g., differentiated, from progenitor cells, such as stem cells. In other case, primary cells or cultured cells derived from primary cells can be used to form cell layers to make synthetic leather. In some embodiments, an engineered cell comprising a molecular switch can be used to form cell layers to make synthetic leather. In some embodiments, cells can be differentiated by contacting the cells with a tissue formation medium.

In some embodiments, the methods described herein provide high-throughput methods that reliably, accurately, and reproducibly scale up to commercial levels the production of synthetic leather. Advantages of the synthetic leather, engineered epidermal equivalent, engineered full thickness skin equivalent and methods of making the same disclosed herein comprise production of customized tissues in a reproducible, high throughput and easily scalable fashion with appealing appearance, texture, thickness, and durability. In some embodiments, the methods described herein can produce increase yields of one or more of an epidermal layer, dermal layer, layered structure or synthetic leather. In some embodiments, increase yields can be at least about 0.5, 0.6, 0.7, 0.8, 0.9, 1, 1.5, 2, 2.5, 3, 3.5, 4, 5, 6, 7, 8, 9, 10, 11, 12, 13, or about 15 times yield compared to a comparable method. In some embodiments, the methods disclosed herein can reduce the cost of the manufacture of synthetic leathers, artificial epidermal layers, artificial dermal layers, layered structures, and products produced therefrom. In some embodiments, the methods disclosed herein can produce uniform thickness synthetic leathers, artificial epidermal layers, artificial dermal layers, layered structures, and products produced therefrom. In some embodiments, the synthetic leathers, artificial epidermal layers, artificial dermal layers, layered structures, and products produced therefrom can have a substantially uniform thickness, length and/or width. In some embodiments, cells in any one or more of an epidermal layer, dermal layers, layered structures can be homogeneously distributed. In some embodiments, cells in any one or more of an epidermal layer, dermal layers, layered structures can be heterogeneously distributed.

### EXAMPLES

### Example 1. Development of an Immortalized Bovine Fibroblast Cell

In some embodiments, an immortalized bovine fibroblast cell line can provide a consistent and sustainable source of cells to support a demand for industrial-scale leather production. In some embodiments, most primary cells can have a finite number of cell doublings known as a Hayflick number, which is correlated to a shortening of a chromosome after every doubling. In some embodiments, upon reaching this number, a cell tends to go through a death phase. In some embodiments, a non-immortalized or primary cell line can have a capacity to generate enough cells for at least 1 million square feet per year of leather. In some embodiments, more hides may need to be generated to meet market demands. In some embodiments, an increase in production can be achieved by immortalization of primary cells. In some embodiments, immortalization of a primary cell can allow a cell to reproduce unlimitedly. In some embodiments, a cell line can be further engineered to improve yield and reduce a cost. In some embodiments, cells can grow in 2D. In some embodiments, growing in 2D can comprise a cell attaching to and growing on tissue culture plastic. In some embodiments, through modifications, cells could be grown in a 3D environment, such as a suspension culture, to increase production efficiency. In some embodiments, a cell can be engineered to proliferate or differentiate without a reliance on expensive components such as growth and cell attachment factors.

In some embodiments, to generate an immortalized bovine fibroblast cell, TERT and Bmi1 genes can be inserted into a plasmid vector. In some embodiments, a plasmid can be transfected into a fibroblast cell line. In some embodiments, a plasmid can contain gfp, so a fluorescent selectable marker can be used to determine successful transfection. In some embodiments, a selected fibroblast line carrying a plasmid can be purified and expanded. In some embodiments, a fibroblast cell line can display an immortalized phenotype, can remain diploid, can secrete a normal extracellular matrix, and can have controlled proliferation. In some embodiments, an immortalized cell line can be frozen for future use.

Alternatively, immortalization can be achieved by expressing exogenous genes, such as SV40 large T antigen (SV40-TAg), telomerase (TERT), BMI1, CCND1, or by intervening with a function of cell cycle regulators such as CDK4, retinoblastoma protein (RB), and p53. SV40-TAg can be a nonstructural protein derived from Simian virus 40 (SV40). Ectopic expression of SV40-TAg can lead to cell transformation by blocking a function of RB and p53. RB and p53 can act as negative cell cycle regulators. RB can regulate DNA replication by repressing a cell cycle progression from G1 to S phase. SV40-TAg can block a function of RB protein, leading to cell cycle progression. p53 can be involved in regulating a G1 to S phase growth cycle progression, apoptosis, or senescence. Interaction between an SV40-TAg and a p53 can interfere with a function of p53, leading to cell cycle progression and cell survival.

In some embodiments, ectopic expression of SV40-TAg can immortalize primary cells from a variety of species and cell types. In some embodiments, ectopic expression of SV40-TAg can immortalize primary cells from a human embryonic fibroblast, a hamster dermal fibroblast, a bovine fetal hepatocyte, a bovine peritoneal macrophages, a bovine dental papilla cells, a bovine embryonic lung cell, or any combination thereof. Disclosed herein are methods comprising using SV40-TAg to immortalize other bovine primary cells such as dermal fibroblasts, pre-adipocytes, amniotic fluid cells, umbilical cord stem cells, and any combination thereof.

In some embodiments, collagen can comprise the most abundant protein in a bovine dermal tissue. In some embodiments, a dermis can be divided into a papillary and a reticular region. In some embodiments, a papillary region can generate a higher quality leather called a grain leather. In some embodiments, a tissue can consist of densely packed collagen fibers (primarily Type I and III) intermixed with elastic fibers (elastin) and extra-fibrillar fibers such as Fibronectin embedded in a polysaccharide gel. In some embodiments, a tanning process can comprise a series of process steps to remove a polysaccharide gel, or other tissue components while chemically cross-linking a collagen. In some embodiments, a cross-linked collagen can then be processed further to be dyed and create a structural feel of leather. In some cases, an expression of SV40-TAg could have an impact on collagen production. In some embodiments, a transduction of SV40-TAg in a human lung fibroblast cell line, WI38, could lead to a reduction in a collagen production. In some embodiments, a transduction of SV40-TAg can have a limited impact on type I and type III collagen production in human ligamentocytes. In some embodiments, a role of SV40 mediated transformation on collagen production could be cell line dependent. In some embodiments, it can be important to demonstrate that a cell line generated by using SV40-TAg can be capable of generating collagens which leads to a production of leather.

### Example 2. Stable Bovine Dermal Fibroblast (BDF) Cell Line Generation

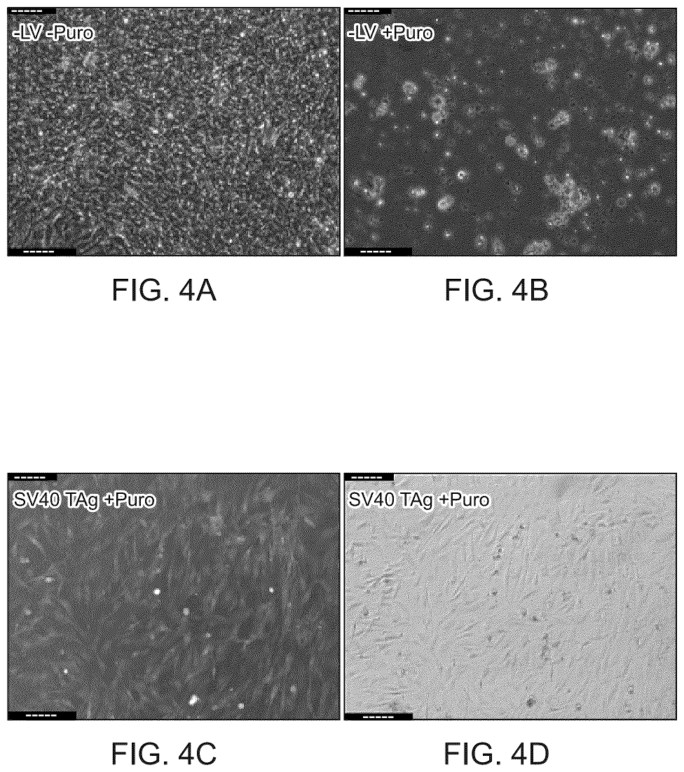

In some embodiments, to generate a stable bovine dermal fibroblast (BDF) cell line, a primary BDF was transduced at passage 5 (P5) using a lentiviral vector to stably express an SV40-TAg-T2A-Puror fragment (Puror=Puromycin resistance gene) under a CMV promoter. A green fluorescent protein (GFP) was expressed under another CMV promoter for visualization (FIG. 3). Post transduction, cells were cultured in 10% Fetal Bovine Serum (FBS) media (Table 1) for three days to allow transgene expression. To select cells carrying an SV40-TAg-T2A-Puror sequence, 0.5 ug/ml of Puromycin was added to the cell culture for two days then Puromycin concentration was increased to 0.75 ug/ml for an additional 7 days. BDF cells transduced with an SV40-TAg-T2A-Puror fragment can survive under puromycin selection and express GFP (FIG. 4A, FIG. 4B, FIG. 4C, and FIG. 4D). SV40-TAg-T2A-Puror transduced cells that survived the Puromycin selection were subsequently named VL-001 cell line and repetitively passaged at 6000 cells/cm² to test their growth potential.

TABLE 1

| Name | Purpose | Components |
|---|---|---|
| HPL media | BDF 3D tissue | DMEM-high glucose |
| | formation media | 1-35% human platelet lysate |
| | | 0.1-15% Acid Citrate Dextrose |
| | | Heparin 0.1-15 IU/mL |
| | | Ascorbic Acid 5-350 ng/mL |
| | | TGF-β1 0.1-15 ng/mL |
| | | Normocin 10-1500 μg/mL |
| 10% FBS | VL-001 2D expansion | DMEM-high glucose |
| media | | 1-35% FBS |
| | | 0.1X-3.7X Antibiotic- |
| | | Antimycotic |
| 20% FBS | VL-001 3D tissue | DMEM-high glucose |
| media | formation media | 1-45% FBS |
| | | 0.1X-27X Non-essential |
| | | amino acid |
| | | 0.1-27X Antibiotic-Antimycotic |

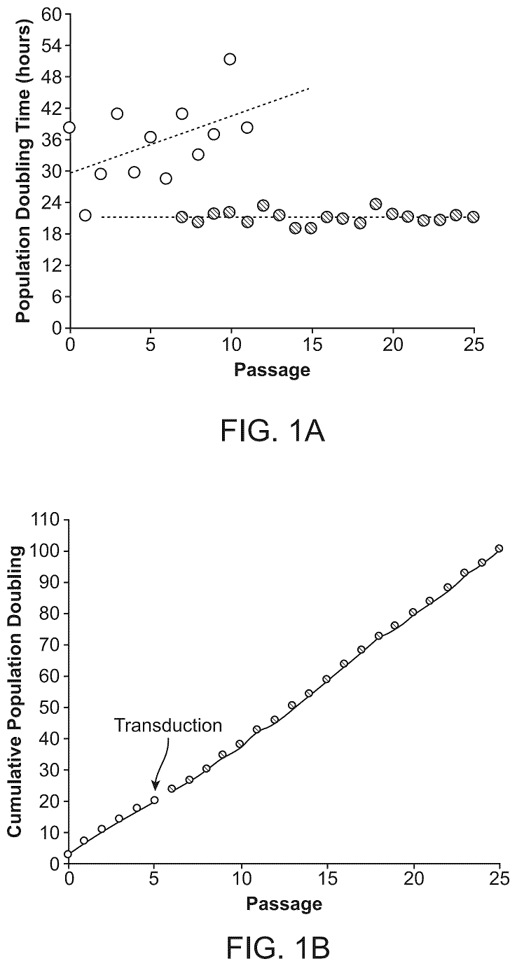

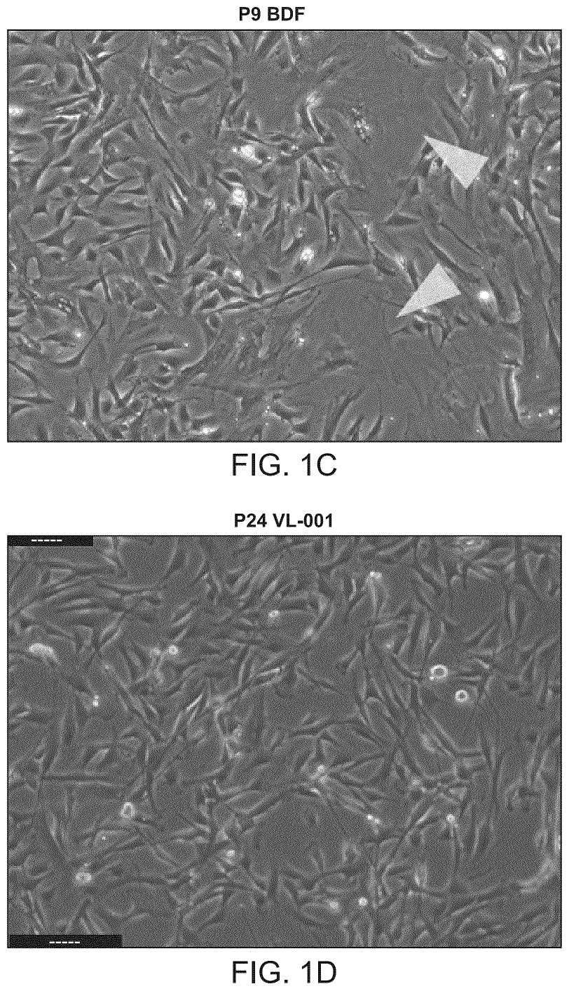

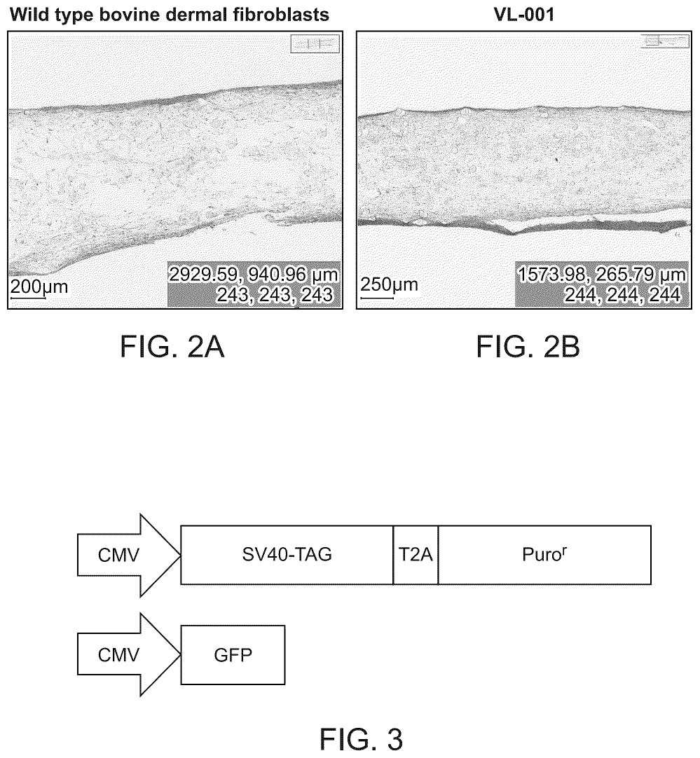

Unmodified BDFs can be expanded in vitro for around 40 population doubling (PD) with an average population doubling time (PDT) of 35.5 hours. A primary BDF was found to change its PDT over multiple passages with a minimal PDT of 21.6 hours and maximum PDT of 51.5 hours from passage 0 to passage 11 (FIG. 2A). In contrast, an immortalized cell line (VL-001) cells were expanded for at least 20 more passages after Puromycin selection to reach a cumulative PD of 100 at passage 25 (FIG. 1B). This number is not indicative of senescence, rather this is when the expansion experiment was stopped. The average PDT of VL-001 cell line (21.2 hours) was 40% shorter than unmodified primary BDFs. VL-001 cell line also exhibited a more consistent PDT among serial passaging. VL-001 cell line has a minimum PDT of 19.1 hours and maximum PDT of 23.7 hours from passage 6 to passage 25 (FIG. 1A). Moreover, no senescent cells were observed in the extensively passaged VL-001 cell line, whereas senescence morphology can be identified in the later passaged unmodified primary BDFs (FIG. 1C—white arrows point to the senescent cells).

### Collagen Production in 2d Culture

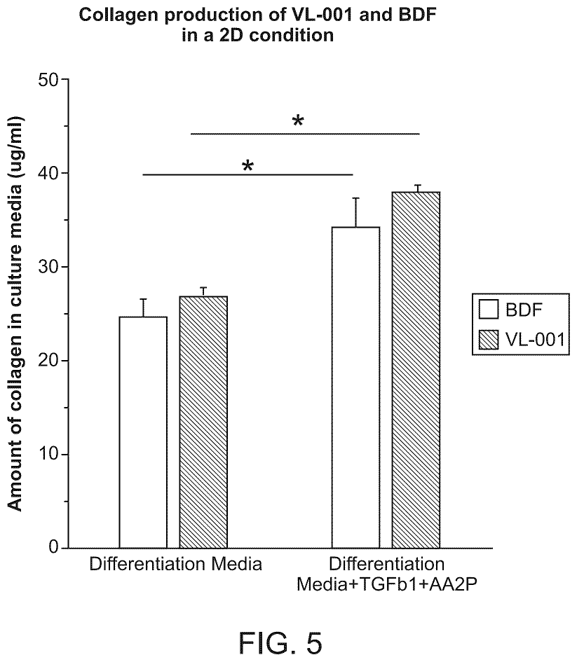

Upon demonstrating that the cells can grow for more PD than the unmodified fibroblast, further experiments focused on whether an immortalized VL-001 fibroblast cell line could produce collagen. It was demonstrated that VL-001 cells could secrete a similar level of collagen in comparison to unmodified primary BDFs when grown in 2D on tissue culture plastic (FIG. 5). Supplementing TGFb1 and ascorbic acid 2-phosphate (AA2P) in a human platelet lysate (HPL)-based media can significantly increase a collagen production of both VL-001 and unmodified BDFs, suggesting that VL-001 has a collagen production machinery similar to unmodified BDFs (FIG. 5).

### Tissue Generation and Collagen Production on a 3D Scaffold

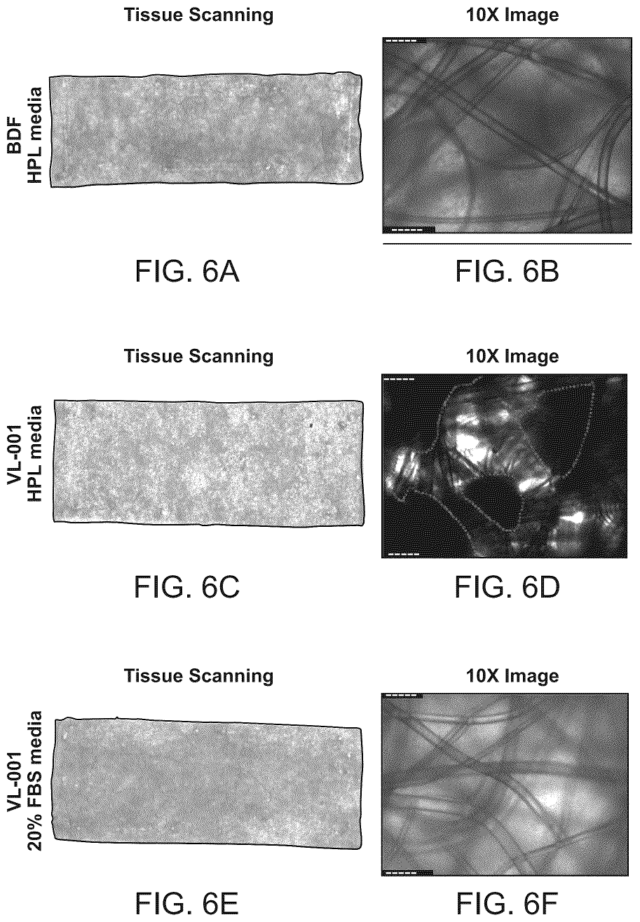

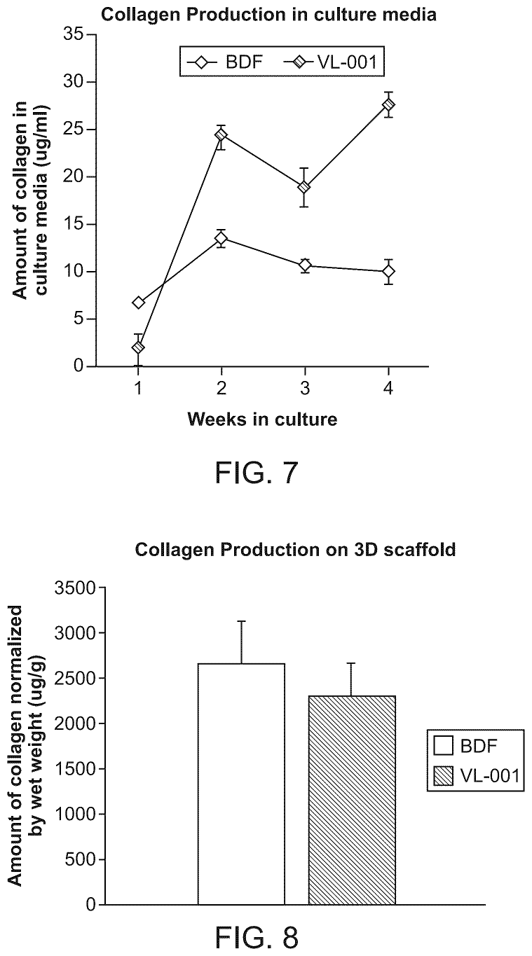

Experiments were performed to determine whether an immortalized VL-001 fibroblast cell line can generate a 3D tissue on a porous polyester non-woven scaffold similar to unmodified primary BDFs. In some cases, unmodified BDFs can form a consistent tissue on a porous scaffold in 4 weeks when cultured in an HPL-based media (Table 1). However, a VL-001 cell line could not; instead, cells formed aggregates and did not spread out on the polyester material when cultured in a same HPL-based media (FIG. 6A, FIG. 6B, FIG. 6C, FIG. 6D, FIG. 6E, and FIG. 6F). After this observation, a second experiment was performed to determine if a similar media formulation used for 2D cell culture of VL-001 (high glucose Dulbecco's Modified Eagles Media—DMEM) with 10% fetal bovine serum could be used for a formation of tissue. Hence, a DMEM with 20% FBS (higher serum is enriched for growth factors and cell attachment proteins) and added 2× non-essential amino acids was used to support tissue formation of VL-001 on a polyester scaffold. By changing a culture media from an HPL media to a 20% FBS media (Table 1), it was found that VL-001 cells could form 3D tissue on a scaffold. VL-001 cells can grow in between supporting fibers similar to unmodified BDFs (FIG. 6A, FIG. 6B, FIG. 6C, FIG. 6D, FIG. 6E, and FIG. 6F). FIG. 6A shows tissue scanning images of BDF cells grown in HPL media. FIG. 6B shows 10× magnification tissue scanning images of BDF cells grown in HPL media 10×. FIG. 6C shows tissue scanning and images of VL-001 cells grown in HPL media. FIG. 6D shows 10× magnification tissue scanning images of VL-001 cells grown in HPL media. FIG. 6E shows tissue scanning images of VL-001 cells grown in 20% FBS media. FIG. 6F shows 10× magnification tissue scanning images of VL-001 cells grown in 20% FBS media. In addition, VL-001 cells cultured in a 20% FBS media can produce free collagen in a culture media (FIG. 7) and deposit collagen fibril in a tissue (FIG. 8) similar to unmodified BDFs cultured in an HPL-based media.

### Suspension Culture

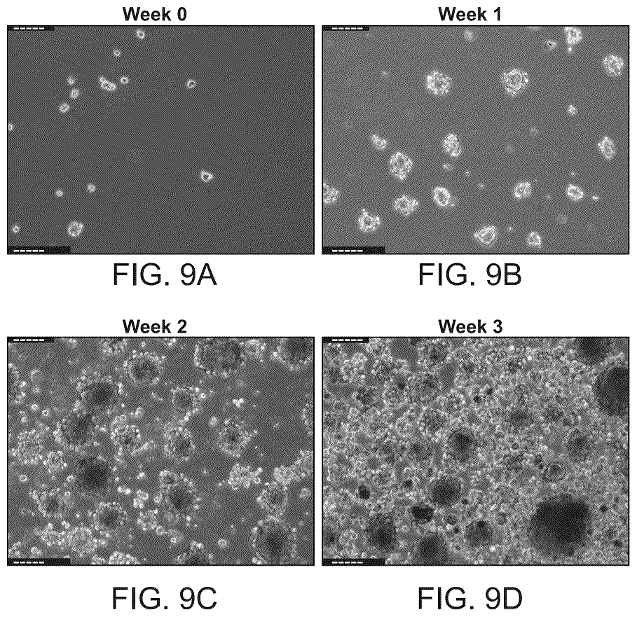

The VL-001 cell line was propagated rapidly in 2D culture conditions. Here, it was shown that the VL-001 cell line could grow in 3D suspension culture as aggregates. Cell aggregation still relied on cell/cell or cell/protein contact. VL-001 cells were seeded on an ultra-low attachment plate at 1000 cells/cm2. A clear aggregate formation was observed after one week of suspension culture. The number and size of the aggregates increased in 3 weeks (FIG. 9A, FIG. 9B, FIG. 9C, and FIG. 9D).

### Example 3. Development of a Cell with a Molecular Switch for Anchorage Independent and Anchorage Dependent Proliferation

To develop a molecular switch, integrin-linked kinase (Ilk) is inserted into a plasmid vector under the control of a temperature sensitive promoter. The temperature sensitive promoter permits expression of Ilk-when cells are grown at 33° C. and inhibits expression when cells are grown at 37° C. to 39° C. The plasmid is transfected into a bovine dermal fibroblast. When Ilk is expressed, the bovine dermal fibroblast displays an anchorage independent proliferation phenotype. The cells are grown in anchorage independent proliferation conditions at a temperature of 33° C. to support cellular proliferation. Once the cells reach a desired density (e.g. 5e⁷ cells/mL), the temperature is changed to 39° C. to abate an expression of Ilk in the dermal fibroblast. The temperature change causes the cells to change to anchorage dependent proliferation and adhere to the scaffold and begin to form tissue. The temperature is reduced to 37° C. to support normal cell proliferation while still reducing an expression of the transfected Ilk. The production of the tissue is used for the generation of synthetic leather.

### Example 4. Development of a Cell with a Molecular Switch for Immortalization

To generate a molecular switch, the hTERT and SV40 genes are inserted into a plasmid vector under the control of a Cre-LoxP system. The Cre-LoxP system permits excision of the hTERT and SV40 genes when cells are grown under conditions where the Cre protein is expressed. The plasmid is transfected into bovine dermal fibroblasts. Under uninduced conditions hTERT and SV40 are expressed and the bovine dermal fibroblasts display an immortalized cellular phenotype. In uninduced conditions, the cells are grown in anchorage independent proliferation conditions. Once the cells reach a desired density (e.g. 5e⁷ cells/mL), an inducer is added to express the Cre recombinase protein and the hTERT and SV40 genes are excised from the dermal fibroblasts. An excision of hTERT and SV40 causes the cells to adhere to the scaffold and begin to form tissue. The production of the tissue is used for the generation of synthetic leather.

### Example 5. Development of an Immortalized Cell with a Molecular Switch

To generate an immortalized bovine fibroblast cell, TERT and CCND1, and a mutant form of CDK4 genes are inserted into a plasmid vector and transfected into a bovine fibroblast cell. The plasmid contains the neo gene (an antibiotic selectable marker) to determine successful transfection. The cells are exposed to geneticin and survivors are selected. The selected fibroblast cell line carrying the plasmid is purified and expanded. The fibroblast cell line displays an immortalized phenotype, remains diploid, secretes a normal extracellular matrix, and has controlled proliferation. Second, to create a molecular switch for an immortalized cells to proliferate in suspension a cyclin D1 gene is cloned with a Tet inducible promoter. The construct is contained in an integrating viral vector and is transfected into an immortalized cell where it integrates into the chromosome. An immortalized cell containing the molecular switch is grown in culture medium with tetracycline which drives an expression of the cyclin D1 gene and thus induces anchorage independent growth. After the cells have reach a density of about 5e⁷ cells/mL the cells are collected, and the medium is replaced with medium that does not contain tetracycline. An absence of an inducer causes the decreased expression of cyclin D1 and anchorage dependent growth on a scaffold. The cells begin to form tissue which is used for the generation of leather.

### Example 6. Preparing Epidermal Layer from Immortalized Bovine Fibroblasts Carrying a Molecular Switch

Immortalized bovine fibroblasts carrying a molecular switch are grown in 0.07 mM Ca²⁺ 154CF medium (Life Technologies) supplemented with man keratinocyte growth supplement. A suspension of immortalized bovine fibroblasts (2.21×10⁵/cm² insert) is seeded on Cellstart CTS (Life Technologies) (or other ECM substrate) coated PET, 0.4-mm inserts (EMD Millipore) in CnT-07 medium (CELLnTEC) or CnT-Prime medium (CELLnTEC) according to manufacturer's protocol.

Day 3 (D3) after seeding, the medium is switched to CnT-02-3D (CELLnTEC) or CnT-3D Barrier (CELLnTEC). On day 4, an immortalized bovine fibroblasts are air exposed by feeding the bottom of an insert with CnT-02-3D or CnT-3D Barrier. From day 4 onward, immortalized bovine fibroblasts are fed daily with CnT-02-3D or CnT-3D Barrier until harvested. Immortalized bovine fibroblasts are grown in a humid (at 100% RH) or dry incubator (at 50% RH) at 37° C. and 5% CO₂. A dial hydrometer (Fisher Scientific) is used to measure incubator humidity. Low incubator humidity is maintained by removal of water pan.

To control for possible changes in osmolarity, medium are refreshed daily. Significant changes in osmolarity are not detected using this protocol, as measured by a Micro Osmometer (Precision Systems). Twelve-well inserts are used for transepithelial electrical resistance (TEER) measurements, light microscopy, and electron microscopy, while six-well inserts are used for transepidermal water loss (TEWL) measurements and immunoblotting.

### Example 7. Culturing an Epidermal Layer

Immortalized bovine fibroblasts carrying a molecular switch are seeded at a density of 2.0-2.5×10⁵ cells/cm² of polyethylene terephthalate (PET) membrane with 0.4 μm pore inserts (EMD Millipore; Cat. No.: MCHT12H48) in CnT-07 medium (CELLnTEC) or CnT-Prime medium (CELLnTEC).

Day 3 (D3) after seeding, the medium is switched to CnT-02-3D (CELLnTEC) or CnT-3D Barrier (CELLnTEC). On day 4, the cells air exposed by feeding the bottom of an insert with CnT-02-3D CnT-3D Barrier. From Day 4 onward, an epidermal layer is fed daily with CnT-02-3D or CnT-3D Barrier until harvested at Day 14.

### Example 8. Preparing Support Substrate

To prepare a 2% agarose solution, 2 g of Ultrapure Low Melting Point (LMP) agarose is dissolved in 100 mL of ultrapure water/buffer solution (1:1, v/v). The buffer solution may be optionally PBS (Dulbecco's phosphate buffered saline 1×) or HBSS (Hanks' balanced salt solution 1×). An agarose solution may be placed in a beaker containing warm water (over 80° C.) and held on the hot plate until an agarose dissolves completely. An agarose solution remains liquid as long as the temperature is above 36° C. Below 36° C., a phase transition occurs, the viscosity increases, and finally an agarose forms a gel.

To prepare agarose support substrate, 10 mL of liquid 2% agarose (temperature >40° C.) may be deposited in a 10 cm diameter Petri dish and evenly spread to form a uniform layer. Agarose is allowed for form a gel at 4° C. in a refrigerator.

### Example 9. Producing a Synthetic Leather Comprising Immortalized Bovine Fibroblasts Carrying a Molecular Switch, Keratinocytes, and Melanocytes

An outline of the protocol can be as follow: a) bringing immortalized bovine fibroblasts carrying a molecular switch and a solution of collagen into contact, then incubating for a sufficient period of time to obtain a contracted collagen matrix in which an immortalized bovine fibroblasts are distributed, constituting a dermis equivalent, b) seeding, with a mixture of keratinocytes and melanocytes, the dermis equivalent obtained in a), and immersion culture in a liquid medium, c) immersion of an entire culture (keratinocytes and melanocytes seeded on the dermis equivalent) obtained in b), and continuation of the culture at an air-liquid interface until a pluristratified epidermis equivalent containing melanocytes, on a dermis equivalent containing fibroblasts in a collagen matrix, constituting a skin equivalent, is obtained.

Step a) can be carried out with collagen type I, in particular of bovine origin, or a mixture of collagens I and III (approximately 30% relative to the final volume of the lattice) in homogeneous suspension. Advantageously, other constituents are added thereto, such as laminin (in particular, from 1% to 15% relative to the final volume), collagen IV (in particular, from 0.3% to 4.5% relative to the final volume) and/or entactin (in particular, from 0.05% to 1% relative to the final volume) so as to obtain a homogeneous suspension. An immortalized bovine fibroblasts are obtained from methods described herein. They are cultured in a suitable medium, and then suspended before mixing with the suspension of collagen and growth factors. The mixture is incubated for 1 to 6 days, for example for 4 or 5 days, at a temperature of approximately 37° C., generally from 36° C. to 37.5° C. Advantageously, the mixture is incubated on a support which does not allow adhesion thereof, in particular which prevents adhesion of the mixture to an edges of the support; such a support may in particular be obtained by prior treatment of its surface, for example by coating said surface with bovine albumin or serum. A collagen gel which is contracted freely in several directions, while discharging the nutritive medium, and in which the fibroblasts are embedded, is thus obtained.

In order to carry out step b), use can be made of keratinocytes originating from skin, for example, from adult skin. The keratinocytes are amplified before seeding according to the technique of Rheinwald and Green (Cell, vol. 6, 331-344, 1975) by culture on a feeder support constituted of 3T3 fibroblasts in a suitable medium, in the presence of growth factors, in particular of amino acids, serum, cholera toxin, insulin, triiodothyronine and pH buffer solution. In particular, such a culture medium may especially contain at least one mitogenic growth factor for keratinocytes (for example, epidermal growth factor (EGF) and/or keratinocyte growth factor (KGF), in particular KGF), insulin, hydrocortisone and, optionally, an antibiotic (for example: gentamycin, amphotericin B).

The melanocytes can be melanocytes originating from young or adult animal skin. They are amplified by culture in a suitable medium, in the absence of phorbol ester, composed of a base medium such as DMEM/F12 or MCDB153 and supplemented with melanocyte-specific growth factors (such as, for example, bFGF, SCF, ET-1, ET3 or aMSH), and in particular in M2 medium (Promocell) or in other medium such as M254 (Cascades Biologics™).

Cell suspensions of melanocytes and of keratinocytes are prepared from these cultures and mixed so as to obtain mixed keratinocyte/melanocyte suspensions. The melanocyte/keratinocyte ratio may be from 1:10 to 2:1 and is generally approximately 1:1. This mixed suspension is deposited on the dermis equivalent. The dermis equivalent is advantageously attached to a support via a biological material such as collagen. The melanocyte/keratinocyte suspension is deposited in a ring or any equivalent means for maintaining it on a delimited surface part. A liquid nutritive medium is added in such a way as to cover the mixture of cells. This medium contains growth factors, in particular EGF and/or KGF. The medium will be replaced regularly and the culture continued as an immersion, generally for a period of from 2 to 10 days, in particular from 5 to 8 days, and approximately 7 days. The medium contains KGF starting from the 2nd day of immersion, and ideally starting from the 4th day of immersion.

The skins are subsequently, in a manner known per se, immersed so as to obtain differentiation of the keratinocytes and formation of a stratified epidermis equivalent. This step c) corresponding to the culture as an immersion at the air-liquid interface is continued until a differentiated structure is obtained, in general approximately 7 days. However, step c) may be continued for a longer period of time, for example for approximately 28 days, while at the same time conserving a skin equivalent having the advantageous characteristics specified in the above text. The nutritive culture medium will be refreshed regularly. The skin equivalent is subsequently removed so as to perform required tests.

### Example 10. Induction of Follicle Formation in Cultured Skin Specimens

Expanded Dermal papilla (DP) cells are mixed with cultured outer root sheath (ORS) cells, washed, and carefully resuspended in 20 ml of sterile phosphate buffered saline (PBS, Sigma) at suitable cell densities. Cultured DP and ORS cells used in each experiment are obtained from different donors, because the different duration of culture for DP and ORS cells do not allow preparation of the two cell types from the same donor. The cell suspension is slowly injected into the dermis of cultured skin pieces 1 day after establishing the culture.

### Example 11. Culturing Hair Follicle Cell Populations

Hair follicles are obtained from an occipital region. Dermal papilla (DP) cells are prepared and cultured as described in Randall et al., A comparison of the culture and growth of dermal papilla cells from hair follicles from non-balding and balding (androgenetic alopecia) scalp. Br J Dermatol 1996: 134: 437-444.).

Briefly the DP of the hair follicles is isolated under a dissecting microscope and transferred individually to a 24-well tissue culture plate (Sarstedt). Cell culture is performed in DMEM, supplemented with 15% FCS (Sigma). After initiation of cell proliferation, cells are cultured to confluency and expanded for two passages. For isolation of outer root sheath (ORS) cells, the middle part of the hair follicles, containing the bulge region, is excised and subjected to mild trypsinization. At least cells of 10 hair follicles are used for each culture. An obtained cells are washed twice in RPMI-1640 medium (Sigma) and subjected to cell culture in standard keratinocyte medium (Epilife, Sigma). Cells are harvested after 1 week of culture.

### Example 12. Tanning Full Thickness Skin Equivalents

Full thickness skin equivalents are tanned by chrome tanning. The first step is ice and sulfuric acid treatment. This opens up the tissue so it can receive the chromium. The chromium is then added along with magnesium oxide.

The process brings the pH level of the full thickness skin equivalents down to around 3. After chromium has worked through the full thickness skin equivalents the tanning liquor is then introduced which brings the pH level up to around 4. This is followed by a warm water bath and then roll pressing to remove excessive liquid. The final stage is then to apply a surface treatment if necessary and then dry the full thickness skin equivalents while stretched out and then re-press when done.

### Example 13. Tanning Artificial Leather

For the following protocol the below definitions are used:
Drum—Acrylic or metal tanning drum
Roller—Roller apparatus used to rotate the tanning drum
Chamber—Heating chamber, with the ability to control the environmental temperature between 25 C to 50 C, where the roller and drum will be placed during operation.
Hide—The harvested bovine skin that will undergo the tanning transformation
Stop: Rubber stopper or parafilm cover that seals the hole of the tanning drum.
X % off—number percent based of weight of hide batch
Required Personal protective equipment (PPE): Chemical splash eye protection, N-95 face mask, nitrile gloves, and a cotton or disposable lab coat.

### Protocol

Personal protective equipment is worn. All equipment is cleaned with a 70% ethanol spray or household cleaner. If using household cleaner, equipment is thoroughly rinsed. All hides that are going to be tanned are gathered and weighed on a lab scale. This weight will determine the amount of water, tanning chemicals, and formic acid used in the worksheet. Once all equipment is dried and ready to use, the tanning process can begin.

The roller is placed inside the incubator chamber and the temperature is adjusted to 24° C. 1000% off water and 2% off prodegreaze into the drum. Manually shake the drum to mix the Prodegreaze into the water. Check the pH of the solution

### Example

Batch weight=100 g
1000% off water=10×100=1000 mL
2% off Prodegreaze=0.02×100=2 g

The hides are placed inside the mixture and the drum sealed using the stopper. The drum is placed on top of the roller and the incubator chamber closed. The mixture is allowed to rotate at 19 RPM for 15 mins.

Note: IBI roller is analog and is determined by the position on the dial. At full speed (19 RPM) the dial will be set to as far as possible, and half speed (9.5 RPM) the dial will be set at half the range.

The hides are removed from the drum and the chemical solution decanted into the appropriate waste container. The drum is rinsed with tap water.

The incubator chamber temperature is kept at 24° C. 1000% off water and 6% off prosoak is added into the drum, and the drum is manually shaken to mix the chemicals together. The hides are added to the drum, the drum is sealed and placed on the roller inside the incubator chamber. The drum is allowed to roll for 30 mins at 19 RPM. The hides are removed from the drum and the chemical solution decanted into the appropriate waste container. 1000% off water and 2% off prosoak are added into the drum, manually shake the drum to mix the chemicals together. The pH of the solution is checked. The hides are added to the drum, the drum is sealed and placed on the roller inside the chamber. The drum is allowed to roll for 30 mins at 19 RPM. The hides are removed from the drum and the chemical solution decanted into the appropriate waste container. The drum is rinsed with tap water.

The incubator chamber temperature is kept at 24° C. 1000% off water, 2% off prospread, and 1 mL of Sodium hydroxide concentrate are added into the drum. The pH of the drum is checked and should be between 11-11.2. The hides are placed into the drum and the drum is sealed with the stopper. The drum is placed onto the roller inside the incubator chamber. The drum is allowed to rotate at 19 RPM for 30 mins and then the drum is allowed to rest for 15 minutes. The hides must be submerged in the solution during the 15 mins rest period. The drum begins rolling for another 15 minutes at 19 RPM, and the pH is checked to ensure it is 10.3 or lower. If not, the drum is allowed to rotate at 19 RPM for another 15 minutes, and the pH is checked again. The 15-minute rotations are repeated until the pH reaches 10.3 or lower.

If the pH does not change after 3 15-minute periods, the hides are removed from the drum and the chemical solution is decanted into the appropriate waste container. The drum is rinsed with tap water. 1000% off water is added into the drum and the hides are placed inside the drum. The solution is mixed at 24° C. for 10 mins at 19 RPM. The hides are removed and the water drained once 10 minutes pass. 1000% off water, and 2% off of Ammonium Chloride are added into the drum and the solution is mechanically mixed until well mixed. The pH is checked.

Hides are placed inside the drum and the drum is placed inside the incubator chamber. The drum is allowed to rotate for 30 minutes at 19 RPM. The hides are removed from the drum and the chemical solution decanted into the appropriate waste container. The drum is rinsed with tap water. 1000% off water is added to the drum and the hides are placed inside. The drum is rotated for 10 minutes at 19 RPM. In the meantime, a bulk solution of 8% formic acid is prepared in a chemical fume hood. The hides are removed from the drum and the water is drained from the drum. 1000% off water is added into the drum, and formic acid is slowly added into the drum until the pH of the solution reaches 4.9. The hides are placed inside the drum and the drum is placed inside the incubator chamber on top of the rollers. The drum is rotated for 30 minutes at 19 RPM.

The hides are removed from the drum and the chemical solution decanted into the appropriate waste container. The drum is rinsed with tap water. 500% off water, 2% off prospread, and 10% off Granofin F90 are added into the drum. The solution is mechanically mixed until the Granofin F90 is mixed well into the water. The pH of the solution is checked. The Granofin F90 is a thick white liquid and may require rigorous mixing.

The hides are placed inside the drum and place the drum inside the incubator chamber on the roller. Set the drum to rotate at 19 RPM for 2 hours and 30 minutes. After 2 and a half hours, the temperature of the incubator chamber is increased to 38° C. and the drum allowed to mix for 1 hour at 19 RPM. After 1 hour, the incubator chamber's temperature is increased to 45° C. and the drum allowed to stand still overnight. The hides must be submerged in the tanning solution during overnight incubation. The pH of the solution is checked after overnight incubation. The hides are removed from the drum and the chemical solution decanted into the appropriate waste container. The drum is rinsed with tap water.

The temperature within the incubator chamber is maintained at 45° C. 1000% off water, 2% off Prospread, and 10% off Pellastol XO is added in the drum and the solution is mixed well. The hides are placed into the drum, the drum is sealed and placed on the roller inside the chamber. The roller is set to 19 RPM and rotated for 15 mins. The following chemicals are added into the drum in the order and at the time intervals listed in Table 2.

TABLE 2

| | | Minutes | |
|---|---|---|---|
| Chemical | % off | inside bath | Speed |
| Lipoderm Lacquer LA | 15 | 15 | 19 RPM |
| Dermaphobe WA-71 | 5 | 15 | |
| Stahlite AL-3 | 5 | 15 | |
| Fabric Softener | 45 | | |
| Water | 1000 | 10 | |
| 8% Formic Acid | pH | 25 | |
| | reaches 3.4 | | |

The hides are removed from the drum and the chemical solution decanted into the appropriate waste container. The drum is rinsed with tap water. 1000% off water is added into the drum and the hides are placed inside the drum. The drum is placed on the roller and the drum is rotated at full speed for 5 mins. The hides are removed and set to dry.

### Example 14. Full Thickness Skin Equivalents

A type I collagen matrix (containing 0.5×10⁶ immortalized bovine fibroblasts) is deposited onto polyethylene terephthalate membranes (BD Biosciences) and allowed to polymerize. After incubation of the polymerized matrix for about 7 days, 1×10⁶ keratinocytes and 0.1×10⁶ melanocytes are seeded onto the matrix, and incubated for a further 7 days. The composite culture is raised to the air-liquid interface and fed from below to induce epidermal differentiation. Full thickness skin equivalents are harvested about 14 days later and either snap frozen in liquid nitrogen or embedded in wax. Melanin quantification can be measured by spectrophotometry and expressed either as melanin content per cell or melanin content per culture (area).

### Example 15. Anchorage Independent Cell Growth

Immortalized cells can be grown anchorage independent in suspension. Cell expansion can occur at about 33° C. and then switched to about 39° C. upon seeding the cells onto the scaffold. The temperature could be reduced to 37° C. for the formation of the tissue or maintained at 39° C. if the cells could tolerate this higher temperature. Tissue can thereafter be modified or tanned as disclosed herein.

### Example 16. Serum Free Media

In some cases, immortalized cells as disclosed herein, can be grown in a medium that is serum free. In some cases, use of a serum free media can result in a slower growth of cells. In some embodiments disclosed herein, a serum free media can be used that does not result in a slower growth of cells. In some embodiments, a serum free media can be completely free of fetal bovine serum (FBS), human platelet lysate (HPL), or any combination thereof.

### Example 17. Single Cell Suspension

In some cases, a cell can be genetically modified to allow growth of a cell in suspension as single cells. In some embodiments, a cell grown in suspension can be grown without cell-cell or cell-protein interactions.

### Example 18. Generation of an Immortalized Fetal Bovine Dermal Fibroblasts Line

Fetal bovine dermal fibroblasts were transfected with the SV40 large T antigen as described in Example 4. The cell line generated (VL-001) has shown the ability to proliferate continuously at a doubling rate of 23.7 hrs. and has been grown to >100 doublings (the experiment was stopped at this doubling). In comparison, the parent primary bovine fibroblast doubling rate is 35.5 hrs. and would senesce by 31 doublings.

The development of an immortalized cell line can potentially impact the ability for the cell to differentiate. Specifically for dermal fibroblast cells, when placed in the right environment they will produce a variety of extracellular matrix (ECM) proteins to create a connective tissue. The primary ECM that is crucial for the development of connective tissue are collagens. These proteins are the key components for being cross-linked in a tanning process to form leather.

To address whether the VL-001 cells can be differentiated to form leather, the following studies were performed. The unique element for this discovery was to be able to generate a cell line that proliferates well and has characteristics of an immortalized cell line but can be differentiated to form a connective tissue that can be tanned to generate leather.

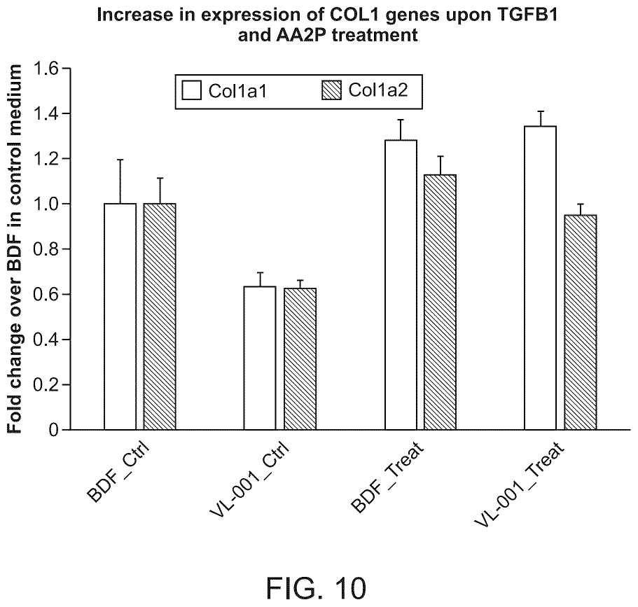

The increase in expression of COL1 genes upon TGFB1 and AA2P treatment is shown in FIG. 10. Type I collagen is the most abundant collagen in dermal tissue and is essential to generate leather. Type I collagen is a rope-like triple-stranded protein composed of 2 alpha1 chains (produced by COL1A1 gene) and 1 alpha2 chain (produced by COL1A2 gene). FIG. 10 shows the increase in expression of COL1 genes upon TGFB1 and AA2P treatment. The expression levels of COL1A1 and COL1A2 gene were up-regulated in both primary Bovine Dermal Fibroblasts (BDF) and the immortalized cell line (VL-001) upon exposure to Transforming Growth Factor beta 1 (TGFB1) and ascorbic acid-2-phosphate (AA2P). Interestingly, VL-001 express less COL1A1 and COL1A2 in the control media without TGFB1 and AA2P stimulation in comparison to BDF in the control media. This result suggests that VL-001 cells might be in a less differentiation condition without TGFB1 and AA2P treatment, which may correlate with the increased proliferation capability of VL-001 cells. Upon TGFB1 and AA2P treatment, VL-001 can increase the expression of COL1A1 and COL1A2 genes to a level similar to the BDF cells. A similar result was observed using a Sircol red assay to measure collagen levels in culture media (see FIG. 5).

### VL-001 Cells Growth on a 3D Scaffold to Generate Dermal Tissue Using a Modified Media Formulation

The next step for comparability to the parent cell line was to determine if the VL-001 cells could be used for the formation of Dermal tissue when grown on a 3D biomaterial scaffold. To address this, the VL-001 cells were tested to see if they could generate a dermal tissue when grown on a Bovine Calf Serum (BCS) coated polylactic acid (PLA) 3D porous scaffold. For coating, PLA scaffolds were immersed in BCS and incubated at 4° C. overnight. Cells were seeded at 1×10⁶ cells/cm² and cultured in a modified media formulation (see below) for 4 weeks. The original tissue formation media developed for BDF cells contained 10% human platelet lysate (hPL), TGFB1, and ascorbic acid-2-phosphate (AA2P). The modified media formulation contained only 5% hPL without TGFB1. This new modified media formulation allowed VL-001 cells to attach and grow in PLA scaffold and reduced the cost by at least 50%. The original and modified media formulations are shown in Table 3.

TABLE 3

| | | |
|---|---|---|
| Ingredient | Original | Modified |

| | | | | |
|---|---|---|---|---|
| DMEM | 700-900 | mL | 750-950 | mL |

| | | |
|---|---|---|
| hPL | 50-100 mL | 0-50 mL |
| | (5%-10%) | (0%-5%) |

| ACD | 0.1-3.5 | mL | N/A | |
|---|---|---|---|---|
| Heparin (2 mg/mL) | 2-3 | mL | 0-1 | mL |
| Ascorbic Acid-2- | 0.4-0.9 | ug | 0.5-1.0 | ug |
| Phosphate | | | | |
| TGF-β1 | 1-2 | ug | 0-1 | ug |
| Normocin | 1-2 | mL | 0-1 | mL |

| | | |
|---|---|---|
| Non-essential | 0x-2x | 1-3x |
| amino acid (NEAA) | | |
| Antibiotic- | 0x-2x | 1-3x |
| Antimycotic | | |

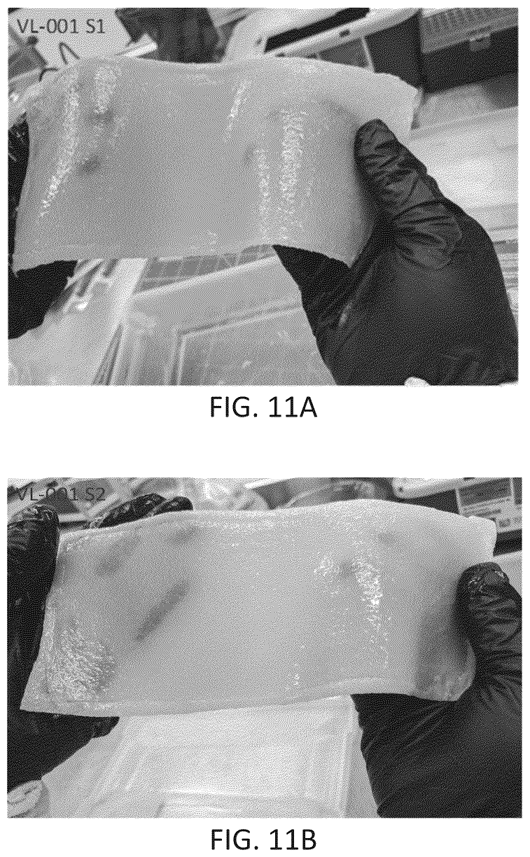

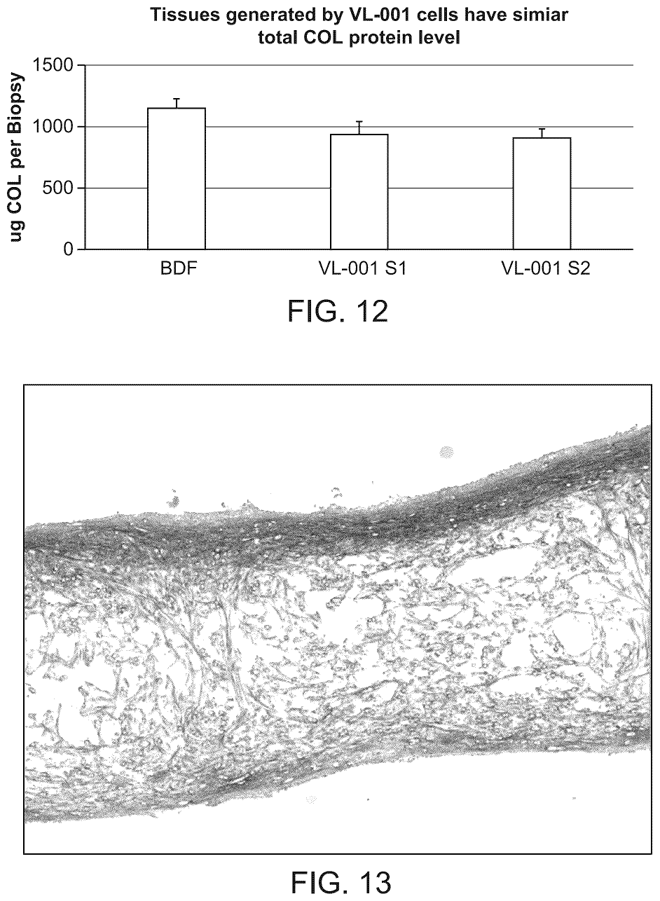

After 4 weeks of culture, VL-001 cells formed continuous tissue within the PLA scaffolds. This process is reproducible, demonstrated by the gross morphology and collagen content. In this experiment, two samples were grown in different bioreactors and exhibited similar morphology after 4 weeks of culture (see FIG. 11A and FIG. 11B). FIG. 11A and FIG. 11B each show an artificial dermal layer grown from an immortalized bovine fibroblast line on a PLA scaffold. Biopsy samples taken from both tissues had similar total collagen content, measured by a Sircol red assay (see FIG. 12). FIG. 12 shows the total collagen content of the VL-001 tissues, using the modified media, is comparable to BDF tissue cultured in the original media.

Tissue biopsies were fixed in a plastic resin and 5 um sections were generated and stained using picrosirius red (PSR) as shown in FIG. 13. FIG. 13 shows that the VL-001 cells could deposit collagen protein throughout the PLA scaffold as indicated by the staining in the image.

### VL-001 Tissue can go Through a Traditional Tanning Process and Generate Leather

Upon completion of the growth of the tissue the engineered dermal tissue was removed from the container and then preserved by immersing in salt or a salt brine solution and stored at 4° C. The hide was then transported to a tannery to be converted into leather. The process consisted of several steps: 1) Presoaking and Soaking to rehydrate the hide and remove the salt, any residual proteins and hyaluronic acid; 2) Liming at a basic pH to remove sulfated glycosaminoglycans (sGAG) and other extraneous tissue/cells from the tissue; 3) Deliming to reduce the pH to around 4.0; 4) Tanning to crosslink the collagen bundles of the tissue; 5) Post-tanning to provide the structural feel (softness and waterproofing) and introduce color to the material; 6) Drying which removed the excess water, but maintained a proper humidity of the material to retain its softness and pliability; and 7) Finishing which provided the final properties such as grain, texture, appearance, etc.

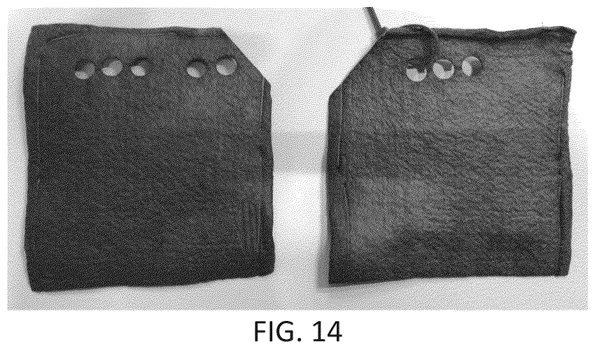

FIG. 14 shows images of VL-001 tissues at the crust phase (after drying to remove the excess water). VL-001 cells were seeded onto Bovine Calf Serum (BCS) coated Poly-L-Lactide Acid (PLA) three-dimensional non-woven scaffolds, grown for 4 weeks in a cell culture media consisting of 5% hPL (human platelet lysate), Heparin (2 mg/L), Non-essential amino acids (1× concentration), ascorbic acid (82 ug/L), Antibiotic-Antimycotic (1× concentration; 100 units/mL of Penicillin, 100 ug/mL of streptomycin, and 250 ng/mL of Gibco Amphotericin B).

### Embodiments

The following are exemplary embodiments of the disclosure herein:
1. A method comprising:
   1) seeding an immortalized animal fibroblast cell onto a scaffold to form an artificial dermal layer;
   2) at least partially decellularizing the artificial dermal layer to form an at least partially decellularized dermal layer; and
   3) tanning the at least partially decellularized artificial dermal layer to form a synthetic leather.
2. The method of embodiment 1, wherein prior to the seeding the immortalized animal fibroblast cell is expanded in culture to form a plurality of immortalized animal fibroblast cells.
3. The method of embodiment 2, wherein the plurality of immortalized animal fibroblast cells can be grown past a Hayflick limit.
4. The method of embodiment 3, wherein the plurality of immortalized animal fibroblast cells can be grown past about 40 cell divisions, about 50 cell divisions, or about 60 cell divisions.
5. The method of embodiment 2, wherein after the expanding, the plurality of immortalized animal fibroblast cells are stored at a temperature below 0° C.
6. The method of embodiment 5, wherein after the storage, the plurality of immortalized animal fibroblast cells are grown in culture before the seeding onto the scaffold.
7. The method of embodiment 1 or 2, wherein the culturing the immortalized animal fibroblast cell comprises expanding the immortalized animal fibroblast cells.
8. The method of any one of embodiments 1-7, wherein the tanning comprises a cross-linking of a collagen in the artificial dermal layer.
9. The method of any one of embodiments 1-8, wherein after the seeding of the immortalized animal fibroblast cell onto the scaffold, the method further comprises culturing the immortalized animal fibroblast cell on the scaffold to form the artificial dermal layer.
10. The method of embodiment 1, wherein the at least partially decellularizing comprises contacting the artificial dermal layer with a salt solution.
11. The method of embodiment 10, wherein the contacting comprises immersing the artificial dermal layer in the salt solution.
12. The method of embodiment 10 or 11, wherein the salt comprises sodium chloride, coarse salt crystals, brine solution, or a combination thereof.
13. The method of embodiment 12, wherein the concentration of the sodium chloride comprises about 36% to about 100%.
14. The method of any one of embodiments 1-13, wherein the tanning comprises a vegetable tanning, a chrome tanning, an aldehyde tanning, a syntan tanning, a bacterial dyeing, or any combination thereof.
15. The method of any one of embodiments 1-14, wherein the animal cell comprises a bovine cell.
16. A method comprising:
   1) seeding an immortalized animal fibroblast cell onto a scaffold to form an artificial dermal layer
   and
   3) tanning the artificial dermal layer to form a synthetic leather.
17. The method of embodiment 16, wherein prior to the seeding the immortalized animal fibroblast cell is expanded in culture to form a plurality of immortalized animal fibroblast cells.
18. The method of embodiment 17, wherein the plurality of immortalized animal fibroblast cells can be grown past the Hayflick limit.
19. The method of embodiment 18, wherein the plurality of immortalized animal fibroblast cells can be grown past about 40 cell divisions, about 50 cell divisions, or about 60 cell divisions.
20. The method of embodiment 17, wherein after the expanding, the plurality of immortalized animal fibroblast cells are stored at a temperature below 0° C.
21. The method of embodiment 20, wherein after the storage, the plurality of immortalized animal fibroblast cells are grown in culture before the seeding onto the scaffold.
22. The method of embodiment 16 or 17, wherein the culturing the immortalized animal fibroblast cell comprises expanding the immortalized animal fibroblast cells.
23. The method of any one of embodiments 16-22, wherein the tanning comprises a cross-linking of a collagen in the artificial dermal layer.
24. The method of any one of embodiments 16-23, wherein the animal cell comprises a bovine cell.
25. A method comprising: seeding a cell onto a scaffold, wherein the cell comprises an exogenous molecule; wherein the exogenous molecule can cause anchorage independent or at least partially anchorage independent proliferation based on a direct or indirect stimulus.
26. The method of embodiment 25, wherein the cell comprises an immortalized cell.
27. The method of embodiment 26, wherein the immortalized cell comprises an immortalized fibroblast cell.
28. The method of embodiment 27, wherein the immortalized fibroblast cell comprises an immortalized bovine fibroblast cell.
29. The method of any one of embodiments 25-28, wherein the molecule comprises an RNA, a DNA, an amino acid, or a protein.
30. The method of embodiment 29, comprising the DNA, wherein the DNA codes for a protein.
31. A method comprising:
   seeding a cell onto a scaffold, wherein the cell comprises an at least partially reversible exogenous molecular switch, an exogenous polynucleotide encoding the at least partially reversible exogenous molecular switch, or both.
32. The method of embodiment 31, wherein the cell is an immortalized cell.
33. The method of embodiment 31 or 32, further comprising before the seeding, proliferating the cell in a first environment, wherein the cell comprises the at least partially reversible exogenous molecular switch, the exogenous polynucleotide encoding the at least partially reversible exogenous molecular switch, or both.
34. The method of any one of embodiments 31-33, wherein the cell is a plurality of cells.
35. The method of embodiment 34, wherein the plurality of cells comprises a plurality of the at least partially reversible exogenous molecular switch, the exogenous polynucleotide encoding the at least partially reversible exogenous molecular switch, or both.
36. The method of embodiment 35, wherein a plurality of the at least partially reversible exogenous molecular switch, the exogenous polynucleotide encoding the at least partially reversible exogenous molecular switch, or both is at least partially on during proliferating and at least partially off during the seeding.
37. The method of embodiment 35, wherein a plurality of the at least partially reversible exogenous molecular switch, the exogenous polynucleotide encoding the at least partially reversible exogenous molecular switch, or both is at least partially off during the proliferating and at least partially on during the seeding.
38. The method of embodiment 33, wherein the at least partially reversible exogenous molecular switch is at least partially on during the proliferating.
39. The method of embodiment 33, wherein the at least partially reversible exogenous molecular switch is at least partially off during the proliferating.
40. The method of any one of embodiments 31-39, wherein the cell is a eukaryotic cell.
41. The method of any one of embodiments 31-39, wherein the cell is selected from the group consisting of a primate cell, bovine cell, ovine cell, porcine cell, equine cell, canine cell, feline cell, rodent cell, bird cell, marsupial, reptile, and lagomorph animal cell.
42. The method of any one of embodiments 31-41, wherein the first environment is in suspension.
43. The method of any one of embodiments 31-42, wherein the at least partially reversible exogenous molecular switch at least partially causes anchorage independent or at least partially anchorage dependent proliferation based on a direct or indirect stimulus.
44. The method of any one of embodiments 31-43, wherein the at least partially reversible exogenous molecular switch is at least partially on during the proliferating and at least partially off during the seeding.
45. The method of any one of embodiments 31-43, wherein the at least partially reversible exogenous molecular switch is at least partially off during the proliferating and at least partially on during the seeding.
46. The method of any one of embodiment 31-45, wherein a presence of a stimuli causes an increased expression or a decreased expression of the exogenous polynucleotide encoding the at least partially reversible exogenous molecular switch.
47. The method of any one of embodiment 31-45, wherein an absence of a stimuli causes an increased expression or a decreased expression of the exogenous polynucleotide encoding the at least partially reversible exogenous molecular switch.
48. The method of any one of embodiments 31-47, wherein the proliferating is in the presence of the stimulus.
49. The method of any one of embodiments 31-47, wherein the proliferating is in the absence of the stimulus.
50. The method of any one of embodiments 31-49, wherein the stimulus is selected from the group consisting of a change in: pH, light, temperature, an electric current, microenvironment, the presence or absence of ions, the level of an ion, the presence of one or more ion type, a change in a mechanical stimulus, a change in a culture medium composition and any combination thereof.
51. The method of embodiment 50, wherein the stimulus comprises the change in the temperature.
52. The method of any one of embodiments 31-51, wherein the at least partially reversible exogenous molecular switch is reversible based on a temperature.
53. The method of any one of embodiments 31-52, wherein the cell is exposed to a temperature of from about 28° C. to about 34° C. during the proliferating.
54. The method of any one of embodiments 31-53, wherein the cell is exposed to a temperature from about 37° C. to about 41° C. following the seeding.
55. The method of any one of embodiments 31-54, wherein the scaffold is at least partially natural or synthetic.
56. The method of any one of embodiments 31-55, wherein the scaffold comprises at least one of the group consisting of a silk, a polylactide, a polyglycolide, a polyester, a polycaprolactone, a chitosan, a hydrogel and a combination thereof.
57. The method of any one of embodiments 31-56, wherein the cell produces an extracellular matrix protein.
58. The method of embodiment 57, wherein the extracellular matrix protein is selected from the group consisting of collagen type I, collagen type III, elastin, fibronectin, laminin, and a combination thereof.
59. The method of any one of embodiments 31-58, further comprising generating at least a portion of a synthetic leather comprising the cell or a portion of tissue developed therefrom.
60. The method of embodiment 59, wherein the synthetic leather comprises at least a portion of the tissue.
61. The method of embodiment 60, wherein the tissue comprises collagen type I.
62. The method of embodiment 31 or 32, further comprising before the seeding, proliferating the cell in a first environment and then directly or indirectly adding the at least partially reversible exogenous molecular switch to the proliferating cell.
63. The method of any one of embodiments 31-62, wherein the cell is seeded at a density from about 50,000 cells/cm² to about 1,000,000 cells/cm².
64. The method of any one of embodiments 31-62, wherein the seeding the cell onto a scaffold comprises seeding one side of the scaffold.
65. The method of embodiment 64, wherein a second side of the scaffold is seeded without flipping the scaffold.
66. The method of any one of embodiments 31-63, wherein the seeding the cell onto a scaffold comprises seeding on more than one side of the scaffold.
67. The method of embodiment 66, wherein the seeding is consecutively or concurrently.
68. The method of embodiment 66, wherein the seeding comprises seeding on one side of the scaffold then flipping the scaffold over and seeding the other side of the scaffold.
69. A method comprising:
   a. transforming a cell into an immortalized cell;
   b. introducing into the immortalized cell an at least partially reversible exogenous molecular switch, an exogenous polynucleotide encoding the at least partially reversible exogenous molecular switch, or both, wherein each of the at least partially reversible exogenous molecular switch an exogenous polynucleotide encoding the at least partially reversible exogenous molecular switch, or both, causes the immortalized cell to proliferate at least partially anchorage independent or at least partially anchorage dependent based on a direct or indirect stimulus; and
   c. proliferating the immortalized cell anchorage independently.
70. The method of embodiment 69, wherein the cell is selected from the group consisting of a primate cell, bovine cell, ovine cell, porcine cell, equine cell, canine cell, feline cell, rodent cell, bird cell, marsupial, reptile, and lagomorph animal cell.
71. The method of embodiment 70, wherein the cell is the bovine cell.
72. The method of any one of embodiments 69-71, wherein the cell is a stem cell.
73. The method of embodiment 72, wherein the stem cell is selected from the group consisting of a mesenchymal stem cell, pluripotent stem cell, induced pluripotent stem cell and an embryonic stem cell.
74. The method of any one of embodiments 69-73, wherein the cell is selected from the group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof.
75. The method of any one of embodiments 69-74, wherein the cell is a fibroblast or a fibroblast-like cell.
76. The method of any one of embodiments 69-75, wherein the transforming comprises increasing or decreasing expression of an oncogene or genes involved in regulation of cells proliferation.
77. The method of any one of embodiments 69-76, wherein the cell expresses a polypeptide or a biologically active fragment thereof encoded by: TERT, Bmi1, or any combination thereof.
78. The method of any one of embodiment 69-77, wherein the immortalized cell expresses a polypeptide or a biologically active fragment thereof encoded by: TERT, CcnD1, Cdk4, SV40 large T antigen, or any combination thereof.
79. The method of any one of embodiments 69-78, further comprising exposing the immortalized cell to the stimulus.
80. The method of embodiment 79, wherein the stimulus is selected from the group consisting of a change in: pH, light, temperature, an electric current, microenvironment, the presence or absence of ions, the level of an ion, the presence of one or more ion type, a change in a mechanical stimulus, a change in a culture medium composition and any combination thereof.
81. The method of embodiment 80, wherein the stimulus comprises the change in the temperature.
82. The method of embodiment 81, wherein the temperature is from about 28° C. to about 34° C.
83. The method of embodiment 82, wherein the stimulus causes the immortalized cell to proliferate at least partially anchorage independent.
84. The method of 83, wherein anchorage independent comprises proliferation in suspension.
85. The method of any one of embodiments 83 or 84, wherein removal of the stimulus causes the immortalized cell to proliferate at least partially anchorage dependent.
86. The method of any one of embodiments 69-85, further comprising exposing the immortalized cell to a second stimulus.
87. The method of embodiment 86, wherein the second stimulus is selected from the group consisting of a change in: pH, light, temperature, an electric current, microenvironment, the presence or absence of ions, the level of an ion, the presence of one or more ion type, a change in a mechanical stimulus, a change in a culture medium composition and any combination thereof.
88. The method of embodiment 87, wherein the second stimulus comprises the change in the temperature.
89. The method of embodiment 88, wherein the temperature is from about 37° C. to about 41° C.
90. The method of embodiment 86, wherein the second stimulus causes the immortalized cell to proliferate at least partially anchorage dependent.
91. The method of embodiment 90, wherein removal of the second stimulus causes the immortalized cell to proliferate at least partially anchorage independent.
92. The method of embodiment 91, wherein anchorage dependent comprises proliferation on a substrate.
93. The method of embodiment 92, further comprising seeding the immortalized cell on the substrate.
94. The method of embodiment 93, wherein the immortalized cell is seeded at a density from about 50,000 cells/cm² to about 1,000,000 cells/cm².
95. The method of embodiment 93, wherein the seeding the immortalized cell on the substrate comprises seeding one side of the substrate.
96. The method of embodiment 95, wherein a second side of the substrate is seeded without flipping the substrate.
97. The method of embodiment 93, wherein the seeding the immortalized cell on the substrate comprises seeding on more than one side of the substrate.
98. The method of embodiment 97, wherein the seeding is consecutively or concurrently.
99. The method of embodiment 97, wherein the seeding comprises seeding on one side of the substrate then flipping the substrate over and seeding the other side of the substrate.
100. The method of embodiment 93, wherein the anchorage dependent proliferation is at least partially on, in, or around the substrate.
101. The method of embodiment 100, wherein the substrate comprises a scaffold.
102. The method of embodiment 101, wherein the scaffold is at least partially natural or synthetic.
103. The method of embodiment 101 or 102, wherein the scaffold comprises a silk, a polylactide, a polyglycolide, a polyester, a polycaprolactone, a chitosan, a hydrogel or a combination thereof.
104. The method of any one of embodiments 31-103, wherein the cell or immortalized cell is modified to have enhanced extracellular matrix production as compared to an otherwise comparable wildtype cell that does not comprise the at least partially reversible exogenous molecular switch or the polynucleotide encoding the at least partially reversible exogenous molecular switch.
105. The method of any one of embodiments 69-104, wherein the extracellular matrix comprises collagen type I, collagen type III, elastin, fibronectin, laminin, or a combination thereof.
106. The method of any one of embodiments 69-105, further comprising engineering a tissue comprising the cell or the immortalized cell.
107. The method of embodiment 106, further comprising tanning the tissue.
108. The method of embodiment 105, wherein the extracellular matrix comprises collagen type I.
109. An engineered cell comprising an at least partially reversible exogenous molecular switch or a polynucleotide encoding the at least partially reversible exogenous molecular switch or a combination thereof, wherein the engineered cell is an immortalized bovine cell.
110. The engineered cell of embodiment 109, wherein the at least partially reversible exogenous molecular switch causes the engineered cell to grow at least partially anchorage independent or at least partially anchorage dependent based on a direct or indirect stimulus.
111. The engineered cell of embodiment 110, wherein proliferation of the engineered cell is determined at least in part by a method selected from the group consisting of manual cell counting, automated cell counting and indirect cell counting.
112. An engineered cell comprising an at least partially reversible exogenous molecular switch or a polynucleotide encoding the at least partially reversible exogenous molecular switch or a combination thereof, wherein the at least partially reversible exogenous molecular switch causes the engineered cell to grow at least partially anchorage independent or at least partially anchorage dependent based on a direct or indirect stimulus.
113. The engineered cell of embodiment 112, wherein proliferation of the engineered cell is determined at least in part by a method selected from the group consisting of manual cell counting, automated cell counting and indirect cell counting.
114. The engineered cell of embodiment 112, wherein the engineered cell is a prokaryotic cell or eukaryotic cell.
115. The engineered cell of embodiment 112, wherein the engineered cell is an animal cell.
116. The engineered cell of embodiment 112, wherein the engineered cell is an isolated cell.
117. The engineered cell of any one of embodiments 112-116, wherein the engineered cell is a non-human cell.
118. The engineered cell of any one of embodiments 112-116, wherein the engineered cell is a human cell.
119. The engineered cell of any one of embodiments 112-117, wherein the engineered cell is selected from the group consisting of a primate cell, bovine cell, ovine cell, porcine cell, equine cell, canine cell, feline cell, rodent cell, bird cell, and lagomorph animal cell.
120. The engineered cell of embodiment 119, wherein the engineered cell is the bovine cell.
121. The engineered cell of any one of embodiments 112-120, wherein the engineered cell is an immortalized cell.
122. The engineered cell of any one of embodiments 119-121, wherein the engineered cell is selected from the group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof.
123. The engineered cell of embodiment 122, wherein the engineered cell is the fibroblast, or a fibroblast-like cell.
124. The engineered cell of any one of embodiments 109-123, wherein the engineered cell is derived from the group consisting of a pluripotent stem cell, a mesenchymal stem cell, induced pluripotent stem cell and an embryonic stem cell.
125. The engineered cell of any one of embodiments 109-123, wherein the engineered cell is derived from a biopsy.
126. The engineered cell of any one of embodiments 109-125, wherein the engineered cell is a cell from a cell line comprising a plurality of cells.
127. The engineered cell of any one of embodiments 109-126, wherein the engineered cell expresses an exogenous oncogene or genes involved in regulation of cells proliferation.
128. The engineered cell of any one of embodiments 109-127, wherein the engineered cell expresses a polypeptide or a biologically active fragment thereof encoded by: TERT, Bmi1, or any combination thereof.
129. The engineered cell of any one of embodiments 109-127, wherein the engineered cell expresses a polypeptide or a biologically active fragment thereof encoded by: TERT, CcnD1, Cdk4, or any combination thereof.
130. The engineered cell of any one of embodiments 109-129, wherein the engineered cell is modified to have enhanced extracellular matrix production as compared to an otherwise comparable wildtype cell that does not comprise the at least partially reversible exogenous molecular switch or the polynucleotide encoding the at least partially reversible exogenous molecular switch.
131. The engineered cell of embodiment 130, wherein the extracellular matrix comprises collagen type I, collagen type III, elastin, fibronectin, laminin, or a combination thereof.
132. The engineered cell of any one of embodiments 109-131, wherein the at least partially reversible exogenous molecular switch or the polynucleotide encoding the at least partially reversible exogenous molecular switch is configured to at least partially increase or decrease expression in response to the stimulus.
133. The engineered cell of embodiment 132, wherein increased expression of the at least partially reversible exogenous molecular switch or the polynucleotide encoding the at least partially reversible exogenous molecular switch causes the at least partial anchorage dependent proliferation.
134. The engineered cell of embodiment 132, wherein increased expression of the at least partially reversible exogenous molecular switch or the polynucleotide encoding the at least partially reversible exogenous molecular switch causes the at least partial anchorage independent proliferation.
135. The engineered cell of embodiment 132, wherein decreased expression of the at least partially reversible exogenous molecular switch or the polynucleotide encoding the at least partially reversible exogenous molecular switch causes the at least partial anchorage dependent proliferation.
136. The engineered cell of embodiment 132, wherein decreased expression of the at least partially reversible exogenous molecular switch or the polynucleotide encoding the at least partially reversible exogenous molecular switch causes the at least partial anchorage independent proliferation.
137. The engineered cell of any one of embodiments 109-136, wherein the at least partial anchorage dependent proliferation consumes more, same or less nutrients or growth factors as compared to anchorage independent proliferation.
138. The engineered cell of any one of embodiments 109-136, wherein the at least partially anchorage independent proliferation consumes more, same, or less nutrients or growth factors as compared to the at least partially anchorage dependent proliferation.
139. The engineered cell of any one of embodiments 109-138, wherein the at least partially reversible exogenous molecular switch or the polynucleotide encoding the at least partially reversible exogenous molecular switch is a single switch, or a plurality of switches.
140. The engineered cell of any one of embodiments 109-139, wherein the polynucleotide encoding the at least partially reversible exogenous molecular switch encodes a selectable marker.
141. The engineered cell of embodiment 140, wherein the selectable marker comprises a fluorescent protein.
142. The engineered cell of any one of embodiments 109-141, wherein the engineered cell comprises a recombinant selectable marker.
143. The engineered cell of embodiment 142, wherein the selectable marker is selected from the group consisting of: an antibiotic resistance gene, a gene encoding a fluorescent protein, and a gene expressing an auxotrophic marker.
144. The engineered cell of any one of embodiments 109-143, comprising the polynucleotide encoding the at least partially reversible exogenous molecular switch wherein the polynucleotide is located in the genome, is extrachromosomal or a combination thereof.
145. The engineered cell of any one of embodiments 109-144, wherein the stimulus is an environmental stimulus.
146. The engineered cell of any one of embodiments 109-145, wherein the stimulus is selected from the group consisting of a change in: pH, light, temperature, an electric current, microenvironment, the presence or absence of ions, the level of an ion, a mechanical stimulus, the presence of one or more ion type, a change in a culture medium composition and any combination thereof.
147. The engineered cell of embodiment 146, wherein the stimulus comprises the change in the temperature.
148. The engineered cell of embodiment 147, wherein the temperature for the at least partially anchorage independent proliferation is from about 28° C. to about 34° C.
149. The engineered cell of embodiment 147 or 148, wherein the temperature for the at least partially anchorage dependent proliferation is from about 37° C. to about 41° C.
150. The engineered cell of any one of embodiments 109-149, wherein presence of the stimulus causes anchorage dependent proliferation.
151. The engineered cell of any one of embodiments 109-149, wherein absence of the stimulus causes anchorage dependent proliferation.
152. The engineered cell of any one of embodiments 109-149, wherein presence of the stimulus causes anchorage independent proliferation.
153. The engineered cell of any one of embodiments 109-149, wherein absence of the stimulus causes anchorage independent proliferation.
154. The engineered cell of any one of embodiments 110-145, wherein the stimulus is selected from the group consisting of a change in a presence, absence, or level of: an antibiotic, a protein, a chemical compound, a salt of any one of these and any combination thereof.
155. The engineered cell of any one of embodiments 110-154, wherein the at least partially anchorage independent proliferation is at least partially a result of an increase in expression of: integrin-linked kinase (ILK), cyclin D1, Cdk4, ST6 N-Acetylgalactosaminide Alpha-2, 6-Sialytransferase 5 (ST6GALNAC5), or a combination thereof.
156. The engineered cell of any one of embodiments 109-155, wherein the engineered cell is grown in a bioreactor.
157. The engineered cell of any one of embodiments 109-156, wherein the at least partially anchorage independent proliferation is at least partially in a suspension and wherein the anchorage dependent proliferation is at least partially on, in, or around a scaffold.
158. The engineered cell of embodiment 157, wherein the scaffold is at least partially natural or synthetic.
159. The engineered cell of embodiment 157 or 158, wherein the scaffold comprises a silk, a polylactide, a polyglycolide, a polyester, a polycaprolactone, a chitosan, a hydrogel or a combination thereof.
160. An isolated tissue comprising the engineered cell of any one of embodiments 109-159.
161. The isolated tissue of embodiment 160, wherein the tissue comprises a plurality of polyester fibers.
162. The isolated tissue of embodiment 160, wherein at least a portion of the tissue is tanned.
163. The isolated tissue of embodiment 162, wherein the at least a portion of the tissue is tanned with a tanning agent comprising: a chromium, an aluminum, a zirconium, a titanium, an iron, a sodium aluminum silicate, a formaldehyde, a glutaraldehyde, an oxazolidine, an isocyanate, a carbodiimide, a polycarbamoyl sulfate, tetrakis hydroxyphosphonium sulfate, a sodium p-[(4,6-dichloro-1,3,5-triazin-2-yl)amino] a benzenesulphonate, a pyrogallol, a catechol, a syntan, or any combination thereof.
164. The isolated tissue of embodiment 162, wherein at least a portion of the tissue further comprises an extracellular matrix.
165. A leather comprising at least a portion of the engineered cell, a derivative thereof, or a progeny thereof of any one of embodiments 109-159, or the isolated tissue of embodiment 160-164.
166. The leather of embodiment 165, wherein the leather is in the form of any one selected from the group consisting of a bag, a belt, a watch strap, a package, a shoe, a boot, a footwear, a glove, a clothing, a vest, a jacket, a pant, a hat, a shirt, an undergarment, a luggage, a clutch, a purse, a ball, a backpack, a wallet, a saddle, a harness, a chap, a whip, furniture, furniture accessories, an upholstery, an automobile car seat, an automobile interior, and any combination thereof.
167. The leather of embodiment 165, wherein the leather comprises a biofabricated material.
168. The leather of embodiment 167, wherein the biofabricated material comprises zonal properties.
169. The engineered cell of any one of embodiments 109-159, wherein the engineered cell comprises any one selected from the group consisting of a c-MycER system, a Tet-on system, a Tet-off system and a combination thereof.
170. The engineered cell of any one of embodiments 109-159 or 169, wherein the engineered cell comprises any one selected from the group consisting of a Cre-LoxP system, TALENS, Zinc Finger, a CRISPR system or a component thereof, and any combination thereof.
171. The engineered cell of any one of embodiments 109-159 or 169-170, wherein the polynucleotide encoding the at least partially reversible exogenous molecular switch comprises DNA, RNA, or a combination thereof.
172. The engineered cell of embodiment 131, wherein the polynucleotide encoding the at least partially reversible exogenous molecular switch comprises the DNA.
173. The engineered cell of embodiment 132 or 171, wherein the polynucleotide encoding the at least partially reversible exogenous molecular switch comprises cDNA.
174. The engineered cell of embodiment 171, wherein the polynucleotide encoding the at least partially reversible exogenous molecular switch comprises the RNA.
175. The engineered cell of embodiment 174, wherein the polynucleotide encoding the at least partially reversible exogenous molecular switch comprises mRNA.
176. The engineered cell of any one of embodiments 109-159 or 169-175, wherein the polynucleotide encoding the at least partially reversible exogenous molecular switch comprises an inducible promotor or operator, wherein the promoter or operator is configured to repress or activate expression of a gene.
177. The engineered cell of embodiment 176, wherein the promotor or operator comprises any one selected from the group consisting of a tetracycline-controlled transcriptional unit, a dexamethasone-controlled transcriptional unit, a doxycycline-controlled transcriptional unit, a C-mycR transcriptional controlled unit and any combination thereof.
178. The engineered cell of any one of embodiments 109-159 or 169-177, wherein the polynucleotide encoding the at least partially reversible exogenous molecular switch is codon optimized.
179. The engineered cell of any one of embodiments 109-159 or 169-178, wherein the polynucleotide encoding the at least partially reversible exogenous molecular switch comprises an epigenetically modified base.
180. The engineered cell of embodiment 179, wherein the epigenetically modified base comprises a pyrimidine.
181. The engineered cell of embodiment 180, wherein the pyrimidine is a cytosine or a thymine.
182. The engineered cell of any one of embodiments 179-181, wherein the epigenetically modified base comprises any one selected from the group consisting of a methylated base, a hydroxymethylated base, a formylated base, and a carboxylic acid containing base.
183. The engineered cell of embodiment 182, wherein the epigenetically modified base comprises the hydroxymethylated base.
184. The engineered cell of any one of embodiments 182-183, wherein the hydroxymethylated base comprises a 5-hydroxymethylated base.
185. The engineered cell of embodiment 184, wherein the 5-hydroxymethylated base comprises a 5-hydroxymethylcytosine.
186. The engineered cell of embodiment 182, wherein the epigenetically modified base comprises the methylated base.
187. The engineered cell of embodiment 186, wherein the methylated base comprises a 5-methylated base.
188. The engineered cell of embodiment 187, wherein the 5-methylated base comprises a 5-methylcytosine.
189. A method comprising contacting the engineered cell of any one of embodiments 109-159 or 169-188 with a stimulus.
190. The method of embodiment 189, wherein the stimulus is an environmental stimulus.
191. The method of any one of embodiments 189-190, wherein the stimulus comprises any one selected from the group consisting of a change in: pH, light, temperature, an electric current, microenvironment, the presence or absence of ions, a change in mechanical stimulus, the level of an ion, the presence of one or more ion type, and any combination thereof.
192. The method of embodiment 191, wherein the stimulus comprises the change in the temperature.
193. The method of embodiment 192, wherein the engineered cells are grown at a temperature for the at least partially anchorage independent proliferation at from about 28° C. to about 34° C.
194. The method of embodiment 193, wherein removal of the stimulus abates the at least partially anchorage independent proliferation.
195. The method of embodiment 192, wherein the engineered cells are grown at a temperature for the at least partially anchorage dependent proliferation at from about 37° C. to about 41° C.
196. The method of embodiment 195, wherein removal of the stimulus abates the at least partially anchorage dependent proliferation.
197. The method of any of embodiments 190-196, wherein the at least partially reversible exogenous molecular switch or the polynucleotide encoding the at least partially reversible exogenous molecular switch is introduced into the engineered cell by transfection, electroporation, or transduction.
198. The method of any of embodiments 190-197, wherein the at least partially reversible exogenous molecular switch or the polynucleotide encoding the at least partially reversible exogenous molecular switch is introduced by any one selected from the group consisting of a vector, wherein the vector is a virus, a viral-like particle, an adeno-associated viral vector, a liposome, a nanoparticle, a plasmid, linear dsDNA and a combination thereof.
199. The method of any one of embodiments 198, wherein the vector comprises the plasmid.
200. The method of any one of embodiments 189-199, wherein proliferation of the engineered cell is determined at least in part by a method selected from the group consisting of manual cell counting, automated cell counting and indirect cell counting.
201. The method of embodiment 200, wherein proliferation of the engineered cell is determined by use of a method selected from the group consisting of a counting chamber, colony forming unit counting, electrical resistance, flow cytometry, image analysis, stereologic cell counting, spectrophotometry, and impedance microbiology.
202. A method comprising tanning at least a portion of a tissue, wherein the tissue comprises the engineered cell of any one of embodiments 109-159 or 169-188.
203. The method of embodiment 202, wherein the tissue comprises a layered structure.
204. The method of embodiment 203, wherein the layered structure comprises any one selected from the group consisting of a dermal layer, an epidermal layer, laminin, fibronectin, collagen and a combination thereof.
205. The method of any one of embodiments 202-204, wherein the tissue comprises any one selected from the group consisting of a fibroblast, a keratinocyte, a corneocyte, a melanocyte, a Langerhans cell, a basal cell, an adipocyte, a smooth muscle cell, an epithelial cell and a combination thereof.
206. A method of selecting or screening for the engineered cell of any one of embodiments 109-159 or 169-188.
207. A synthetic leather, which prior to tanning comprises a portion of a tissue, wherein the tissue comprises the engineered cell of any one of embodiments 109-159 or 169-188.
208. The synthetic leather of embodiment 207, wherein the tissue is at least partially subjected to further processing.
209. The synthetic leather of embodiments 208, wherein the further processing is selected from the group consisting of tanning, preserving, soaking, bating, pickling, depickling, thinning, retanning, lubricating, crusting, wetting, sammying, shaving, rechroming, neutralizing, dyeing, fatliquoring, filling, stripping, stuffing, whitening, fixating, setting, drying, conditioning, milling, staking, buffing, finishing, oiling, brushing, padding, impregnating, spraying, roller coating, curtain coating, polishing, plating, embossing, ironing, glazing, tumbling, and any combination thereof.
210. The synthetic leather of embodiment 207, wherein the synthetic leather comprises a biofabricated material, wherein the biofabricated material comprises the engineered cell.
211. The synthetic leather of embodiment 210, wherein the biofabricated material comprises zonal properties.
212. A culture vessel comprising the engineered cell of any one of embodiments 109-159 or 169-188.
213. The culture vessel of embodiment 212, wherein the culture vessel comprises any one selected from the group consisting of a plastic, a metal, a glass and a combination thereof.
214. The culture vessel of embodiment 212, wherein the culture vessel comprises an agent that causes the engineered cell to adhere to at least a portion of the culture vessel.
215. The culture vessel of embodiment 214, wherein the agent comprises poly-L-lysine.
216. A manufacturing facility comprising the engineered cell of any one of embodiments 109-159 or 169-188.
217. A kit comprising the engineered cell of any one of embodiments 109-159 or 169-188.
218. The kit of embodiment 217, further comprising a growth medium.
219. The kit of any one of embodiments 217 or 218, wherein the kit further comprises instructions for use.
220. A method comprising: seeding a transfected or transduced isolated cell onto a scaffold to form an artificial dermal layer and contacting the transfected or transduced isolated cell with a medium, wherein the transfected or transduced isolated cell comprises an exogenous polynucleotide, wherein:
   a. the exogenous polynucleotide encodes:
      (i) a polypeptide which interacts with a tumor suppressor protein or fragment thereof and alters an activity of the tumor suppressor protein or fragment thereof, wherein the activity of the tumor suppressor protein or fragment thereof can be measured by an in vitro assay, or
      (ii) a polynucleotide that encodes the polypeptide which interacts with the tumor suppressor protein or fragment thereof,
   b. the transfected or transduced isolated cell when in contact with the medium has:
      (i) increased collagen production, relative to an otherwise identical cell that is not contacted with the medium,
      (ii) at least partially increased differentiation, relative to an otherwise identical cell that is not contacted with the medium, or
      (iii) any combination of (i) and (ii).
221. The method of embodiment 220, further comprising at least partially decellularizing the artificial dermal layer to form an at least partially decellularized artificial dermal layer.
222. The method of embodiment 221, further comprising tanning the at least partially decellularized artificial dermal layer to form a synthetic leather.
223. The method of embodiment 220, wherein the exogenous polynucleotide codes for an SV40 large T antigen (SV40-TAg) polypeptide, a telomerase (TERT) protein, a Bmi-1 protein, a cyclin D1 protein, a biologically active fragment of any of these, or any combination thereof.
224. The method of embodiment 223, wherein the exogenous polynucleotide comprises an SV40-TAg gene, a TERT gene, a BMI1 gene, a CCND1 gene, a transcript of any of these, an exon of any of these, or any combination thereof.
225. The method of embodiment 223, wherein the exogenous polynucleotide codes for an SV40 large T antigen (SV40-TAg) protein, a biologically active fragment thereof, or any combination thereof.
226. The method of embodiment 223, wherein the exogenous polynucleotide codes for an TERT protein, a biologically active fragment thereof, or any combination thereof.
227. The method of embodiment 223, wherein the exogenous polynucleotide codes for a Bmi-1 protein, a biologically active fragment thereof, or any combination thereof.
228. The method of embodiment 223, wherein the exogenous polynucleotide codes for a cyclin D1 protein, a biologically active fragment thereof, or any combination thereof.
229. The method of embodiment 220, wherein the tumor suppressor protein or biologically active fragment thereof is a cyclin dependent kinase 4, a retinoblastoma, a p53, a biologically active fragment of any of these, or any combination thereof.
230. The method of any one of embodiments 220-229, wherein after transfection or transduction, a cell growth cycle of the transfected or transduced isolated cell is at least partially uninhibited.
231. The method of embodiment 230, wherein after transfection or transduction, the transfected or transduced isolated cell can be grown past about 50 cell divisions, about 70 cell divisions, about 90 cell divisions or about 100 cell divisions.
232. The method of any one of embodiments 220-231, wherein the medium comprises a growth medium, a tissue formation medium, or a combination thereof.
233. The method of embodiment 232, wherein after transfection or transduction with the exogenous polynucleotide, the transfected or transduced isolated cell (a) proliferates, (b) avoids senescence, or (c) both, when present in the growth medium.
234. The method of any one of embodiments 232 or 233, wherein prior to the seeding, the transfected or transduced isolated cell is expanded in the growth medium to form a plurality of transfected or transduced cells.
235. The method of embodiment 233, wherein prior to the seeding the transfected or transduced isolated cell is expanded in a container that at least partially inhibits cellular adherence.
236. The method of embodiment 232, wherein after the seeding, the transfected or transduced isolated cell is contacted with a tissue formation medium.
237. The method of embodiment 232, wherein the growth medium comprises a growth factor, a buffer, a salt, a sugar, an amino acid, a lipid, a vitamin, an ECM protein, a fragment of any of these, or any combination thereof.
238. The method of embodiment 237, comprising the salt, wherein the salt comprises an inorganic salt.
239. The method of embodiment 238, wherein the inorganic salt comprises about 0.2 g/L Calcium Chloride, about 0.0001 g/L Ferric Nitrate·9H₂O, about 0.09767 g/L Magnesium Sulfate (anhydrous), about 0.4 g/L Potassium Chloride, about 3.7 g/L Sodium Bicarbonate, about 6.4 g/L Sodium Chloride, about 0.109 g/L Sodium Phosphate Monobasic (anhydrous), or any combination thereof.
240. The method of embodiment 237, comprising the amino acid, wherein the amino acid comprises about 0.084 g/L L-Arginine·HCl, about 0.0626 g/L L-Cystine·2HCl, about 0.03 g/L Glycine, about 0.042 g/L L-Histidine·HCl·H2O, about 0.105 g/L L-Isoleucine, about 0.105 g/L L-Leucine, about 1.46 g/L L-Lysine·HCl, about 0.03 g/L L-Methionine, about 0.066 g/L L-Phenylalanine, about 0.042 g/L L-Serine, about 0.095 g/L L-Threonine, about 0.016 g/L L-Tryptophan, about 0.12037 g/L L-Tyrosine 2Na·2H₂O, about 0.094 g/L L-Valine, about 0.584 g/L L-Glutamine, a stereoisomer of any of these, a salt of any of these, or any combination thereof.
241. The method of embodiment 237, comprising the vitamin, wherein the vitamin comprises about 0.004 g/L Choline Chloride, about 0.004 g/L Folic Acid, about 0.0072 g/L myo-Inositol, about 0.004 g/L Niacinamide, about 0.004 g/L D-Pantothenic Acid (hemicalcium), about 0 g/L Pyridoxal·HCl, about 0.004 g/L Pyridoxine·HCl, about 0.0004 g/L Riboflavin, about 0.004 g/L Thiamine·HCl, a stereoisomer of any of these, a salt of any of these, or any combination thereof.
242. The method of embodiment 237, comprising the sugar, wherein the sugar comprises D-Glucose, a stereoisomer thereof, a salt thereof, or any combination thereof.
243. The method of embodiment 237, comprising the pH indicator, wherein the pH indicator comprises about 0.0159 g/L Phenol Red·Na, about 0.11 g/L Pyruvic Acid·Na, a stereoisomer of any of these, a salt of any of these, or any combination thereof.
244. The method of embodiment 237, wherein the growth medium comprises an amino acid, a vitamin, an inorganic salt, a fetal bovine serum, an antibiotic, an antimycotic, or any combination thereof.
245. The method of embodiment 232, comprising the tissue formation medium, wherein the tissue formation medium comprises a growth factor, a buffer, a salt, a sugar, a vitamin, an amino acid, a lipid, a mineral, an inorganic salt, an ECM protein, a human platelet lysate, an acid citrate dextrose, a heparin, an ascorbic acid, a TGF-β1, a normocin, a serum, a serum alternative, a non-essential amino acid, an antibiotic, an antimycotic, or any combination thereof.
246. The method of embodiment 245, wherein the tissue formation medium further comprises from about 0.10% to about 40% of a serum, a serum alternative, or a combination thereof.
247. The method of embodiment 245, comprising the amino acid, wherein the amino acid comprises a Glycine, an Alanine, an L-Arginine hydrochloride, an L-Asparagine-H2O, an L-Aspartic acid, an L-Cysteine hydrochloride-H₂O, an L-Cystine 2HCl, an L-Glutamic Acid, an L-Glutamine, an L-Histidine hydrochloride-H₂O, an L-Isoleucine, an L-Leucine, an L-Lysine hydrochloride, an L-Methionine, an L-Phenylalanine, an L-Proline, an L-Serine, an L-Threonine, an L-Tryptophan, an L-Tyrosine disodium salt dihydrate, an L-Valine, a stereoisomer of any of these, a salt of any of these, or any combination thereof.
248. The method of embodiment 245, comprising the vitamin, wherein the vitamin comprises a biotin, a choline chloride, a D-Calcium pantothenate, a Folic acid, a Niacinamide, a Pyridoxine hydrochloride, a Riboflavin, a Thiamine hydrochloride, a Vitamin B12, an i-Inositol, a stereoisomer of any of these, a salt of any of these, or any combination thereof.
249. The method of embodiment 245, comprising the salt, wherein the salt comprises a calcium chloride, a cupric sulfate, a ferric nitrate, a ferric sulfate, a magnesium chloride, a magnesium sulfate, a potassium chloride, a sodium bicarbonate, a sodium chloride, a sodium phosphate dibasic, a sodium phosphate monobasic, a zinc sulfate, a stereoisomer of any of these, a salt of any of these, or any combination thereof.
250. The method of embodiment 245, wherein the medium further comprises a D-Glucose (Dextrose), a hypoxanthine Na, a linoleic acid, a lipoic acid, a phenol red, a putrescine 2HCl, a Sodium pyruvate, a thymidine, a stereoisomer of any of these, a salt of any of these, or any combination thereof.
251. The method of embodiment 245, comprising the serum alternative, wherein the serum alternative contains substantially no animal derived products, is xeno-free, or a combination thereof.
252. The method of embodiment 251, wherein the serum alternative comprises a growth factor, an insulin, a transferrin, a cytokine, an essential amino acid, nonessential amino acids, a protein, an extracellular matrix protein, a laminin, a fibronectin, a vitronectin, a cell adhesion peptide, an RGD, extracellular matrix fragments, a hormone, a collagen, an albumin, a lipid, a glycoprotein, a protein fragment, or any combination thereof.
253. The method of embodiment 245, comprising the serum, wherein the serum comprises a fetal bovine serum (FBS), a horse serum, a fetal calf serum or any combination thereof.
254. The method of any one of embodiments 232-253, wherein the medium does not comprise TGF beta.
255. The method of any one of embodiments 245-253, wherein after the seeding, the transfected or transduced isolated cell is contacted with the tissue formation medium.
256. The method of any one of embodiments 236, or 245-256, wherein the transfected or transduced isolated cell (a) at least partially increases production of a collagen, (b) has an at least partially arrested cell growth, or (c) both, when present in the tissue formation medium, relative to an otherwise comparable transfected or transduced isolated cell that has not been contacted with the tissue formation medium.
257. The method of any one of embodiments 236, or 245-256, wherein the transfected or transduced isolated cell is at least partially differentiated when contacted with the tissue formation medium.
258. The method of embodiment 1, wherein after the seeding, the transfected or transduced isolated cell is contacted with a medium comprising L-ascorbic acid 2-phosphate (AA2P), the TGFB1, a salt thereof, a biologically active fragment thereof, or a combination of any of these.
259. The method of embodiment 258, wherein the transfected or transduced isolated cell (a) at least partially increases production of collagen, (b) has an at least partially arrested cell growth, or (c) both, when present in the medium comprising the AA2P, the TGFB1, the salt thereof, the biologically active fragment thereof, or the combination, relative to an otherwise comparable medium lacking the AA2P, the salt thereof, the TGFB1, the biologically active fragment thereof, or the combination.
260. The method of embodiment 258, wherein the transfected or transduced isolated cell is at least partially differentiated when contacted with the medium.
261. The method of any one of embodiments 220-260, wherein the transfected or transduced isolated cell comprises an isolated immortalized cell, an isolated reprogrammed cell, an isolated progenitor cell, an isolated mesenchymal stem cell, or any combination thereof.
262. The method of any one of embodiments 220-261, wherein the transfected or transduced isolated cell comprises an isolated fibroblast cell, an isolated mesenchymal cell, an isolated stem cell derived cell, an isolated umbilical cord stem cell, an isolated amniotic tissue cell, an isolated scar tissue cell, or any combination thereof.
263. The method of any one of embodiments 220-262, wherein the cell displays markers of collagen production.
264. The method of any one of embodiments 220-263, wherein the cell can be isolated by flow cytometry.
265. The method of any one of embodiments 220-264, wherein the transfected or transduced isolated cell is isolated from a bovine, a non-human mammal, a reptile, a bird, a shark, a kangaroo, a fish, or an eel.
266. The method of embodiment 265, wherein the transfected or transduced isolated cell is isolated from the bovine, and wherein the transfected or transduced isolated cell comprises a bovine fibroblast cell.
267. The method of embodiment 265, wherein the transfected or transduced isolated cell is isolated from the reptile, and wherein the transfected or transduced isolated cell comprises a turtle cell, a snake cell, a lizard cell, an amphibian cell, a crocodile cell, or an alligator cell.
268. The method of embodiment 265, wherein the transfected or transduced isolated cell is isolated from the non-human mammal, wherein the transfected or transduced isolated cell comprises an antelope cell, a bear cell, a beaver cell, a bison cell, a boar cell, a camel cell, a caribou cell, a cat cell, a cattle cell, a deer cell, a dog cell, an elephant cell, an elk cell, a fox cell, a giraffe cell, a goat cell, a hare cell, a horse cell, an ibex cell, a lion cell, a llama cell, a lynx cell, a mink cell, a moose cell, an oxen cell, a peccary cell, a pig cell, a rabbit cell, a rhino cell, a seal cell, a sheep cell, a squirrel cell, a tiger cell, a whale cell, a wolf cell, a yak cell, or a zebra cell.
269. The method of embodiment 265, wherein the transfected or transduced isolated cell is isolated from the bird, wherein the transfected or transduced isolated cell comprises a chicken cell, a duck cell, an emu cell, a goose cell, a grouse cell, an ostrich cell, a pheasant cell, a pigeon cell, a quail cell, or a turkey cell.
270. The method of any one of embodiments 220-267, wherein the transfected or transduced isolated cell was derived from a scar tissue, an umbilical cord, or a combination thereof.
271. The method of embodiment 220, wherein prior to the seeding, the transfected or transduced isolated cell was selected for the presence of the exogenous polynucleotide.
272. The method of embodiment 271, wherein the selection for the presence of the exogenous polynucleotide comprises an antibiotic selection.
273. The method of embodiment 272, wherein the antibiotic comprises a puromycin.
274. The method of any one of embodiments 220-273, wherein the scaffold comprises a porous material.
275. The method of embodiment 274, wherein the transfected or transduced isolated cell is engrafted to the scaffold.
276. The method of embodiment 274 or 275, wherein the scaffold comprises a synthetic material, a non-synthetic material, or a combination thereof.
277. The method of embodiment 276, comprising the non-synthetic material, wherein the non-synthetic material comprises a silk, a natural tissue adhesive, a fibrin glue, a collagen, a basement membrane protein, an extracellular matrix, or a combination thereof.
278. The method of embodiment 276, comprising the synthetic material, wherein the scaffold comprises a polyethylene (PE), a polypropylene (PP), a Polyethylene terephthalate (PET), a Polyamide 6,6 (PA 6,6), a Polyamide 11 (PA 11), a Polyvinylidene fluoride (PVDF), a Polyethylene furanoate (PEF), a Polyurethane (PU), a Polyhydroxyalkanoate (PHA), a Polyhydroxybutyrate (PHB), a Poly(3-hydroxybutyrate-co-3-hydroxyvalerate) (PHBV), a Polylactic acid (PLA), a Polycaprolactone (PCL), a Polybutylene succinate (PBS), a Poly(glycolic) acid (PGA), a Poly(lactic-co-glycolic acid (PLGA), a Polyvinyl Alcohol (PVOH), an Alginate, a Copolymer PEGylated fibrin (P-fibrin), a Poly(glycerol sebacate) (PGS), a poly(L-lactic acid) (PLLA), a Poly(lactic-coglycolic acid) (PLGA), a Poly-D,L-lactic acid/polyethylene glycol/poly-D,L-lactic acid (PDLLA-PEG), a hyaluronic acid (HA), a carbon nanotube, a Thermoplastic Starch, a Lyocell/Tencel (Cellulose), a Cotton, a Bast fiber, a Viscose Bamboo, a TiO₂ nanofiber, a cellulose material, a hydrogel material, an alginate, a gelatin, a nylon, a polyester, a silk, a material cross-linked with a cell adhesion peptide, a material cross-linked with growth factors, or any combination thereof.
279. An engineered cell comprising an exogenous molecule, wherein the exogenous molecule at least partially alters the activity of pRB or P53, wherein the engineered cell is an immortalized bovine cell.
280. The engineered cell of embodiment 279, wherein the at least partially alters comprises an inhibition.
281. The engineered cell of embodiment 280, wherein the inhibition comprises a competitive inhibition.
282. The engineered cell of embodiment 281, wherein the competitive inhibition does not function on a non-transformed cell.
283. The engineered cell of embodiment 282, wherein the competitive inhibition only functions on a genetically modified cell.
284. The engineered cell of embodiment 279, wherein the molecule comprises an RNA, a DNA, a small molecule, a salt thereof, a polypeptide, a hormone, or a biologically active fragment thereof.
285. The engineered cell of embodiment 284, comprising the DNA, wherein the DNA codes for a polypeptide or a biologically active fragment thereof.
286. The engineered cell of embodiment 284, comprising the RNA, wherein the RNA comprises a mRNA, a siRNA, or a miRNA.
287. A method comprising contacting a transfected or transduced isolated cell comprising an exogenous polynucleotide with a medium comprising from about 0.1% to about 40% FBS, wherein the transfected or transduced isolated cell when present in the medium comprising the FBS (a) produces collagen, (b) has an at least partially arrested cell growth, or (c) both, and wherein after transfection or transduction the activity of at least one of pRB or P53 is at least partially altered in the transfected or transduced isolated cell.
288. The method of embodiment 287, wherein the medium comprises about 20% FBS.
289. The method of embodiment 287, wherein the medium further comprises L-ascorbic acid 2-phosphate (AA2P), a salt thereof, transforming growth factor beta 1 (TGFB1), a biologically active fragment thereof, or any combination thereof.
290. The method of any one of embodiments 287-289, wherein the exogenous polynucleotide codes for an SV40 large T antigen, an hTERT, a Bmi-1, a cyclin D1, a biologically active fragment of any of these, or any combination thereof.
291. The method of any one of embodiments 287-290, wherein the transfected or transduced isolated cell is a fibroblast cell.
292. The method of any one of embodiments 287-291, wherein the transfected or transduced isolated cell is isolated from a bovine, a non-human mammal, a reptile, a bird, a shark, a kangaroo, a fish, or an eel.
293. The method of embodiment 222, wherein the synthetic leather is made into the form of any item selected from the group consisting of a bag, a belt, a watch strap, a package, a shoe, a boot, a footwear, a glove, a clothing, a vest, a jacket, a pant, a hat, a shirt, an undergarment, a luggage, a clutch, a purse, a ball, a backpack, a wallet, a saddle, a harness, a chap, a whip, an item of furniture, a furniture accessory, an upholstery, an automobile car seat, an automobile interior, and any combination thereof.
294. A composition comprising: (i) a transfected or transduced isolated cell comprising an exogenous polynucleotide wherein after transfection or transduction the activity of at least one of pRB or P53 is at least partially altered in the transfected or transduced isolated cell, (ii) a scaffold, and (iii) a medium, wherein the transfected or transduced isolated cell is at least partially contained on, in, or around the scaffold.
295. An artificial dermal layer comprising: (i) a transfected or transduced isolated cell comprising an exogenous polynucleotide and (ii) a scaffold, wherein after transfection or transduction the activity of at least one of pRB or P53 is at least partially altered in the transfected or transduced isolated cell, and wherein the transfected or transduced isolated cell is at least partially contained on, in, or around the scaffold.
296. The artificial dermal layer of embodiment 295, wherein at least a portion of the artificial dermal layer is at least partially decellularized.
297. The artificial dermal layer of embodiment 296, wherein at least a portion of the at least partially decellularized tissue is tanned to form a synthetic leather.
298. A composition comprising an immortalized fibroblast cell and a medium comprising an effective amount of: FBS, L-Ascorbic acid 2-phosphate (AA2P) or a salt thereof, Transforming Growth Factor Beta 1 (TGFB1) or a biologically active fragment thereof, or any combination thereof, wherein the effective amount is sufficient to induce a reporter cell comprising a transfected or transduced polynucleotide to:
   (a) increase (i) production of collagen; (ii) secretion of collagen; or (iii) both, and
   (b) arrest cell growth in the reporter cell,
when the reporter cell is present in the medium, relative to an otherwise comparable medium lacking the effective amount of the FBS, the L-Ascorbic acid 2-phosphate (AA2P) or the salt thereof, the Transforming Growth Factor Beta 1 (TGFB1) or the biologically active fragment thereof, or the combination, as determined by:
   (a) transfecting or transducing a cell with a polynucleotide coding for SV40 large T antigen, a biologically active fragment thereof, TERT, a biologically active fragment thereof, or any combination thereof,
   (b) growing the cell in the medium and the otherwise comparable medium;
   (c) comparing a growth rate of the cells grown in the medium relative to the otherwise comparable medium; and
   (d) comparing production of collagen produced in the medium relative to the otherwise comparable medium.
299. A composition comprising: (i) a transfected or transduced isolated cell comprising an exogenous polynucleotide wherein after transfection or transduction the activity of at least one of pRB or P53 is at least partially altered in the transfected or transduced isolated cell and (ii) the medium of embodiment 0.
300. A composition comprising: (i) a transfected or transduced isolated cell comprising an exogenous polynucleotide wherein after transfection or transduction the activity of at least one of pRB or P53 is at least partially altered in the transfected or transduced isolated cell and (ii) the medium of embodiment 0.
301. A method comprising:
   1) seeding an immortalized animal fibroblast cell onto a scaffold to form an artificial dermal layer;
   2) at least partially decellularizing the artificial dermal layer to form an at least partially decellularized artificial dermal layer; and 3) tanning the at least partially decellularized artificial dermal layer to form a synthetic leather.
302. The method of embodiment 301, wherein prior to the seeding the immortalized animal fibroblast cell is expanded in culture to form a plurality of immortalized animal fibroblast cells.
303. The method of embodiment 302, wherein the plurality of immortalized animal fibroblast cells can be grown past a Hayflick limit.
304. The method of embodiment 303, wherein the plurality of immortalized animal fibroblast cells can be grown past about 40 cell divisions, about 50 cell divisions, or about 60 cell divisions.
305. The method of embodiment 302, wherein after the expanding, the plurality of immortalized animal fibroblast cells are stored at a temperature below 0° C.
306. The method of embodiment 305, wherein after the storage, the plurality of immortalized animal fibroblast cells are grown in culture before the seeding.
307. The method of any one of embodiments 301-306, wherein the tanning comprises a cross-linking of a collagen in the artificial dermal layer.
308. The method of any one of embodiments 301-307, wherein after the seeding of the immortalized animal fibroblast cell onto the scaffold, the method further comprises culturing the immortalized animal fibroblast cell on the scaffold to form the artificial dermal layer.
309. The method of embodiment 301, wherein the at least partially decellularizing comprises contacting the artificial dermal layer with a salt solution, a crystalline salt, or a combination thereof.
310. The method of any one of embodiments 301-309, wherein the animal fibroblast cell comprises a bovine fibroblast cell.
311. The method of embodiment 309, wherein the contacting comprises immersing the artificial dermal layer in the salt solution.
312. The method of embodiment 309 or 311, wherein the salt solution comprises sodium chloride.
313. The method of embodiment 312, wherein the concentration of the sodium chloride comprises about 30-40% sodium chloride.
314. The method of any one of embodiments 301-313, wherein the tanning comprises a vegetable tanning, a chrome tanning, an aldehyde tanning, a syntan tanning, a bacterial dyeing, or any combination thereof.
315. A method comprising:
   a) seeding an immortalized animal fibroblast cell onto a scaffold to form an artificial dermal layer; and
   b) tanning the artificial dermal layer to form a synthetic leather.
316. The method of embodiment 315, wherein prior to the seeding the immortalized animal fibroblast cell is expanded in culture to form a plurality of immortalized animal fibroblast cells.
317. The method of embodiment 316, wherein the plurality of immortalized animal fibroblast cells can be grown past the Hayflick limit.
318. The method of embodiment 317, wherein the plurality of immortalized animal fibroblast cells can be grown past about 40 cell divisions, about 50 cell divisions, or about 60 cell divisions.
319. The method of embodiment 316, wherein after the expanding, the plurality of immortalized animal fibroblast cells are stored at a temperature below 0° C.
320. The method of embodiment 319, wherein after the storage, the plurality of immortalized animal fibroblast cells are grown in culture before the seeding onto the scaffold.
321. The method of embodiment 320, wherein the culturing the immortalized animal fibroblast cell comprises expanding the immortalized animal fibroblast cells.
322. The method of any one of embodiments 315-321, wherein the tanning comprises a cross-linking of a collagen in the artificial dermal layer.
323. The method of any one of embodiments 315-322, wherein the animal fibroblast cell comprises a bovine fibroblast cell.
324. A method comprising: seeding a cell onto a scaffold, wherein the cell comprises an exogenous molecule; wherein the exogenous molecule can cause anchorage independent or at least partially anchorage independent proliferation based on a direct or indirect stimulus.
325. The method of embodiment 324, wherein the cell comprises an immortalized cell.
326. The method of embodiment 325, wherein the immortalized cell comprises an immortalized fibroblast cell.
327. The method of embodiment 326, wherein the immortalized fibroblast cell comprises an immortalized bovine fibroblast cell.
328. The method of any one of embodiments 324-327, wherein the molecule comprises an RNA, a DNA, or a protein.
329. The method of embodiment 328, comprising the DNA, wherein the DNA codes for a protein.
330. A method comprising:
   seeding a cell onto a scaffold, wherein the cell comprises an at least partially reversible exogenous molecular switch, an exogenous polynucleotide encoding the at least partially reversible exogenous molecular switch, or both.
331. The method of embodiment 330, wherein the cell is an immortalized cell.
332. The method of embodiment 330 or 331, further comprising before the seeding, proliferating the cell in a first environment.
333. The method of any one of embodiments 330-332, wherein the cell is a plurality of cells.
334. The method of embodiment 333, wherein the plurality of cells comprises a plurality of the at least partially reversible exogenous molecular switch, the exogenous polynucleotide encoding the at least partially reversible exogenous molecular switch, or both.
335. The method of embodiment 334, wherein the plurality of the at least partially reversible exogenous molecular switch, the exogenous polynucleotide encoding the at least partially reversible exogenous molecular switch, or both is at least partially on during proliferating and at least partially off during the seeding.
336. The method of embodiment 334, wherein a plurality of the at least partially reversible exogenous molecular switch, the exogenous polynucleotide encoding the at least partially reversible exogenous molecular switch, or both is at least partially off during the proliferating and at least partially on during the seeding.
337. The method of embodiment 332, wherein the at least partially reversible exogenous molecular switch is at least partially on during the proliferating.
338. The method of embodiment 332, wherein the at least partially reversible exogenous molecular switch is at least partially off during the proliferating.
339. The method of any one of embodiments 330-338, wherein the cell is a eukaryotic cell.
340. The method of any one of embodiments 330-338, wherein the cell is selected from the group consisting of a primate cell, a bovine cell, a ovine cell, a porcine cell, an equine cell, a canine cell, a feline cell, a rodent cell, a bird cell, a marsupial cell, a reptile cell, and a lagomorph animal cell.
341. The method of any one of embodiments 332-340, wherein the first environment is in suspension.
342. The method of any one of embodiments 330-341, wherein the at least partially reversible exogenous molecular switch at least partially causes anchorage independent or at least partially anchorage dependent proliferation based on a direct or indirect stimulus.
343. The method of any one of embodiments 330-342, wherein the at least partially reversible exogenous molecular switch is at least partially on during the proliferating and at least partially off during the seeding.
344. The method of any one of embodiments 330-342, wherein the at least partially reversible exogenous molecular switch is at least partially off during the proliferating and at least partially on during the seeding.
345. The method of any one of embodiments 330-344, wherein a presence of a stimuli causes an increased expression or a decreased expression of the exogenous polynucleotide encoding the at least partially reversible exogenous molecular switch.
346. The method of any one of embodiment 330-344, wherein an absence of a stimuli causes an increased expression or a decreased expression of the exogenous polynucleotide encoding the at least partially reversible exogenous molecular switch.
347. The method of any one of embodiments 330-346, wherein the proliferating is in the presence of the stimulus.
348. The method of any one of embodiments 340-346, wherein the proliferating is in the absence of the stimulus.
349. The method of any one of embodiments 330-348, wherein the stimulus is selected from the group consisting of a change in: pH, light, temperature, an electric current, microenvironment, magnetic charge, the presence or absence of ions, the level of an ion, the presence of one or more ion type, a change in a mechanical stimulus, a small molecule, an antibiotic, a hormone, a change in a culture medium composition and any combination thereof.
350. The method of embodiment 349, wherein the stimulus comprises the change in the temperature.
351. The method of any one of embodiments 330-350, wherein the at least partially reversible exogenous molecular switch is reversible based on a temperature.
352. The method of any one of embodiments 330-351, wherein the cell is exposed to a temperature of from about 28° C. to about 34° C. during the proliferating.
353. The method of any one of embodiments 330-352, wherein the cell is exposed to a temperature from about 37° C. to about 41° C. following the seeding.
354. The method of any one of embodiments 330-353, wherein the scaffold is at least partially natural or synthetic.
355. The method of any one of embodiments 330-354, wherein the scaffold comprises at least one of the group consisting of a silk, a polylactide, a polyglycolide, a polyester, a polycaprolactone, a chitosan, a hydrogel, and any combination thereof.
356. The method of any one of embodiments 330-355, wherein the cell produces an extracellular matrix protein.
357. The method of embodiment 356, wherein the extracellular matrix protein is selected from the group consisting of collagen type I, collagen type III, elastin, fibronectin, laminin, glycosaminoglycan, hyaluronic acid, and any combination thereof.
358. The method of any one of embodiments 330-357, further comprising generating at least a portion of a synthetic leather comprising the cell or a portion of a tissue developed therefrom.
359. The method of embodiment 358, wherein the synthetic leather comprises at least a portion of the tissue.
360. The method of embodiment 369, wherein the tissue comprises collagen type I.
361. The method of embodiment 330 or 331, further comprising before the seeding, proliferating the cell in a first environment and then directly or indirectly adding the at least partially reversible exogenous molecular switch to the proliferating cell.
362. The method of any one of embodiments 330-361, wherein the cell is seeded at a density from about 30,000 cells/cm² to about 1,000,000 cells/cm². 363. The method of any one of embodiments 330-362, wherein the seeding the cell onto a scaffold comprises seeding one side of the scaffold.
364. The method of embodiment 363, wherein a second side of the scaffold is seeded without flipping the scaffold.
365. The method of any one of embodiments 330-362, wherein the seeding the cell onto a scaffold comprises seeding on more than one side of the scaffold.
366. The method of embodiment 365, wherein the seeding is consecutively or concurrently.
367. The method of embodiment 365, wherein the seeding comprises seeding on one side of the scaffold then flipping the scaffold over and seeding the other side of the scaffold.
368. A method comprising:
   a. transforming a cell into an immortalized cell;
   b. introducing into the immortalized cell an at least partially reversible exogenous molecular switch, an exogenous polynucleotide encoding the at least partially reversible exogenous molecular switch, or both, wherein each of the at least partially reversible exogenous molecular switch an exogenous polynucleotide encoding the at least partially reversible exogenous molecular switch, or both, causes the immortalized cell to proliferate at least partially anchorage independent or at least partially anchorage dependent based on a direct or indirect stimulus; and
   c. proliferating the immortalized cell anchorage independently.
369. The method of embodiment 368, wherein the cell is selected from the group consisting of a primate cell, a bovine cell, a ovine cell, a porcine cell, an equine cell, a canine cell, a feline cell, a rodent cell, a bird cell, a marsupial cell, a reptile cell, and a lagomorph animal cell.
370. The method of embodiment 369, wherein the cell is the bovine cell.
371. The method of any one of embodiments 368-370, wherein the cell is a stem cell.
372. The method of embodiment 371, wherein the stem cell is selected from the group consisting of a mesenchymal stem cell, a pluripotent stem cell, an induced pluripotent stem cell and an embryonic stem cell.
373. The method of any one of embodiments 368-372, wherein the cell is selected from the group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof.
374. The method of any one of embodiments 368-373, wherein the cell is a fibroblast or a fibroblast-like cell.
375. The method of any one of embodiments 368-374, wherein the transforming comprises increasing or decreasing expression of an oncogene or genes involved in regulation of cells proliferation.
376. The method of any one of embodiments 368-375, wherein the cell expresses a polypeptide or a biologically active fragment thereof encoded by: TERT, Bmi1, or any combination thereof.
377. The method of any one of embodiment 368-376, wherein the immortalized cell expresses a polypeptide or a biologically active fragment thereof encoded by: TERT, CcnD1, Cdk4, or any combination thereof.
378. The method of any one of embodiments 368-376, further comprising exposing the immortalized cell to the stimulus.
379. The method of embodiment 378, wherein the stimulus is selected from the group consisting of a change in: pH, light, temperature, an electric current, microenvironment, the presence or absence of ions, the level of an ion, the presence of one or more ion type, a change in a mechanical stimulus, a change in a culture medium composition and any combination thereof.
380. The method of embodiment 379, wherein the stimulus comprises the change in the temperature.
381. The method of embodiment 380, wherein the temperature is from about 28° C. to about 34° C.
382. The method of embodiment 381, wherein the stimulus causes the immortalized cell to proliferate at least partially anchorage independent.
383. The method of 382, wherein anchorage independent comprises proliferation in suspension.
384. The method of any one of embodiments 382 or 383, wherein removal of the stimulus causes the immortalized cell to proliferate at least partially anchorage dependent.
385. The method of any one of embodiments 368-383, further comprising exposing the immortalized cell to a second stimulus.
386. The method of embodiment 385, wherein the second stimulus is selected from the group consisting of a change in: pH, light, temperature, an electric current, microenvironment, magnetic charge, the presence or absence of ions, the level of an ion, the presence of one or more ion type, a change in a mechanical stimulus, a small molecule, an antibiotic, a hormone, a change in a culture medium composition and any combination thereof.
387. The method of embodiment 386, wherein the second stimulus comprises the change in the temperature.
388. The method of embodiment 387, wherein the temperature is from about 37° C. to about 41° C.
389. The method of embodiment 381, wherein the second stimulus causes the immortalized cell to proliferate at least partially anchorage dependent.
390. The method of embodiment 389, wherein removal of the second stimulus causes the immortalized cell to proliferate at least partially anchorage independent.
391. The method of embodiment 390, wherein anchorage dependent comprises proliferation on a substrate.
392. The method of embodiment 391, further comprising seeding the immortalized cell on the substrate.
393. The method of embodiment 392, wherein the immortalized cell is seeded at a density from about 30,000 cells/cm² to about 1,000,000 cells/cm². 394. The method of embodiment 392, wherein the seeding the immortalized cell on the substrate comprises seeding one side of the substrate.
395. The method of embodiment 394, wherein a second side of the substrate is seeded without flipping the substrate.
396. The method of embodiment 392, wherein the seeding the immortalized cell on the substrate comprises seeding on more than one side of the substrate.
397. The method of embodiment 396, wherein the seeding is consecutively or concurrently.
398. The method of embodiment 396, wherein the seeding comprises seeding on one side of the substrate then flipping the substrate over and seeding the other side of the substrate.
399. The method of embodiment 392, wherein the anchorage dependent proliferation is at least partially on, in, or around the substrate.
400. The method of embodiment 399, wherein the substrate comprises a scaffold.
401. The method of embodiment 400, wherein the scaffold is at least partially natural or synthetic.
402. The method of embodiment 400 or 301, wherein the scaffold comprises a silk, a polylactide, a polyglycolide, a polyester, a polycaprolactone, a chitosan, a hydrogel or a combination thereof.
403. The method of any one of embodiments 390-402, wherein the cell or immortalized cell is modified to have enhanced extracellular matrix production as compared to an otherwise comparable wildtype cell that does not comprise the at least partially reversible exogenous molecular switch or the polynucleotide encoding the at least partially reversible exogenous molecular switch.
404. The method of any one of embodiments 369-403, wherein the extracellular matrix comprises collagen type I, collagen type III, elastin, fibronectin, laminin, or a combination thereof.
405. The method of any one of embodiments 368-304, further comprising engineering a tissue comprising the cell or the immortalized cell.
406. The method of embodiment 405, further comprising tanning the tissue.
407. The method of embodiment 404, wherein the extracellular matrix comprises collagen type I.
408. An engineered cell comprising an at least partially reversible exogenous molecular switch or a polynucleotide encoding the at least partially reversible exogenous molecular switch or a combination thereof, wherein the engineered cell is an immortalized bovine cell.
409. The engineered cell of embodiment 408, wherein the at least partially reversible exogenous molecular switch causes the engineered cell to grow at least partially anchorage independent or at least partially anchorage dependent based on a direct or indirect stimulus.
410. The engineered cell of embodiment 409, wherein proliferation of the engineered cell is determined at least in part by a method selected from the group consisting of manual cell counting, automated cell counting and indirect cell counting.
411. An engineered cell comprising an at least partially reversible exogenous molecular switch or a polynucleotide encoding the at least partially reversible exogenous molecular switch or a combination thereof, wherein the at least partially reversible exogenous molecular switch causes the engineered cell to grow at least partially anchorage independent or at least partially anchorage dependent based on a direct or indirect stimulus.
412. The engineered cell of embodiment 411, wherein proliferation of the engineered cell is determined at least in part by a method selected from the group consisting of manual cell counting, automated cell counting and indirect cell counting.
413. The engineered cell of embodiment 411, wherein the engineered cell is a prokaryotic cell or eukaryotic cell.
414. The engineered cell of embodiment 411, wherein the engineered cell is an animal cell.
415. The engineered cell of embodiment 411, wherein the engineered cell is an isolated cell, an enriched population of cells, a purified population, or any combination thereof.
416. The engineered cell of any one of embodiments 411-415, wherein the engineered cell is a non-human cell.
417. The engineered cell of any one of embodiments 411-415, wherein the engineered cell is a human cell.
418. The engineered cell of any one of embodiments 411-416, wherein the engineered cell is selected from the group consisting of a primate cell, bovine cell, ovine cell, porcine cell, equine cell, canine cell, feline cell, rodent cell, bird cell, and lagomorph animal cell.
419. The engineered cell of embodiment 418, wherein the engineered cell is the bovine cell.
420. The engineered cell of any one of embodiments 411-419, wherein the engineered cell is an immortalized cell.
421. The engineered cell of any one of embodiments 418-420, wherein the engineered cell is selected from the group consisting of a fibroblast, an animal fat tissue derived cell, an umbilical cord derived cell, a keratinocyte, a corneocyte, a melanocyte, a Langerhans cell, a basal cell, an adipocyte, a smooth muscle cell, an epithelial cell, a cuboidal cell, a columnar cell, a collagen producing cell, and a combination thereof.
422. The engineered cell of embodiment 421, wherein the engineered cell is the fibroblast, or a fibroblast-like cell.
423. The engineered cell of any one of embodiments 408-422, wherein the engineered cell is derived from the group consisting of a pluripotent stem cell, a mesenchymal stem cell, induced pluripotent stem cell and an embryonic stem cell.
424. The engineered cell of any one of embodiments 408-422, wherein the engineered cell is derived from a biopsy.
425. The engineered cell of any one of embodiments 408-424, wherein the engineered cell is a cell from a cell line comprising a plurality of cells.
426. The engineered cell of any one of embodiments 408-425, wherein the engineered cell express an exogenous oncogene or genes involved in regulation of cells proliferation.
427. The engineered cell of any one of embodiments 408-426, wherein the engineered cell expresses a polypeptide or a biologically active fragment thereof encoded by: TERT, Bmi1, or any combination thereof.
428. The engineered cell of any one of embodiments 408-426, wherein the engineered cell expresses a polypeptide or a biologically active fragment thereof encoded by: TERT, CcnD1, Cdk4, or any combination thereof.
429. The engineered cell of any one of embodiments 408-428, wherein the engineered cell is modified to have enhanced extracellular matrix production as compared to an otherwise comparable wildtype cell that does not comprise the at least partially reversible exogenous molecular switch or the polynucleotide encoding the at least partially reversible exogenous molecular switch.
430. The engineered cell of embodiment 429, wherein the extracellular matrix comprises collagen type I, collagen type III, elastin, fibronectin, laminin, or a combination thereof.
431. The engineered cell of any one of embodiments 408-430, wherein the at least partially reversible exogenous molecular switch or the polynucleotide encoding the at least partially reversible exogenous molecular switch is configured to at least partially increase or decrease expression in response to the stimulus.
432. The engineered cell of embodiment 431, wherein increased expression of the at least partially reversible exogenous molecular switch or the polynucleotide encoding the at least partially reversible exogenous molecular switch causes the at least partial anchorage dependent proliferation.
433. The engineered cell of embodiment 431, wherein increased expression of the at least partially reversible exogenous molecular switch or the polynucleotide encoding the at least partially reversible exogenous molecular switch causes the at least partial anchorage independent proliferation.
434. The engineered cell of embodiment 431, wherein decreased expression of the at least partially reversible exogenous molecular switch or the polynucleotide encoding the at least partially reversible exogenous molecular switch causes the at least partial anchorage dependent proliferation.
435. The engineered cell of embodiment 431, wherein decreased expression of the at least partially reversible exogenous molecular switch or the polynucleotide encoding the at least partially reversible exogenous molecular switch causes the at least partial anchorage independent proliferation.
436. The engineered cell of any one of embodiments 408-435, wherein the at least partial anchorage dependent proliferation consumes more, same or less nutrients or growth factors as compared to anchorage independent proliferation.
437. The engineered cell of any one of embodiments 408-435, wherein the at least partially anchorage independent proliferation consumes more, same, or less nutrients or growth factors as compared to the at least partially anchorage dependent proliferation.
438. The engineered cell of any one of embodiments 408-437, wherein the at least partially reversible exogenous molecular switch or the polynucleotide encoding the at least partially reversible exogenous molecular switch is a single switch, or a plurality of switches.
439. The engineered cell of any one of embodiments 408-438, wherein the polynucleotide encoding the at least partially reversible exogenous molecular switch encodes a selectable marker.
440. The engineered cell of embodiment 439, wherein the selectable marker comprises a fluorescent protein.
441. The engineered cell of any one of embodiments 408-438, wherein the engineered cell comprises a recombinant selectable marker.
442. The engineered cell of embodiment 441, wherein the selectable marker is selected from the group consisting of: an antibiotic resistance gene, a gene encoding a fluorescent protein, and a gene expressing an auxotrophic marker.
443. The engineered cell of any one of embodiments 408-442, comprising the polynucleotide encoding the at least partially reversible exogenous molecular switch wherein the polynucleotide is located in the genome, is extrachromosomal or a combination thereof.
444. The engineered cell of any one of embodiments 409-443, wherein the stimulus is an environmental stimulus.
445. The engineered cell of any one of embodiments 409-444, wherein the stimulus is selected from the group consisting of a change in: pH, light, temperature, an electric current, microenvironment, the presence or absence of ions, the level of an ion, a mechanical stimulus, the presence of one or more ion type, a change in a culture medium composition and any combination thereof.
446. The engineered cell of embodiment 445, wherein the stimulus comprises the change in the temperature.
447. The engineered cell of embodiment 446, wherein the temperature for the at least partially anchorage independent proliferation is from about 28° C. to about 34° C.
448. The engineered cell of embodiment 446 or 447, wherein the temperature for the at least partially anchorage dependent proliferation is from about 37° C. to about 41° C.
449. The engineered cell of any one of embodiments 408-448, wherein presence of the stimulus causes anchorage dependent proliferation.
450. The engineered cell of any one of embodiments 408-448, wherein absence of the stimulus causes anchorage dependent proliferation.
451. The engineered cell of any one of embodiments 408-448, wherein presence of the stimulus causes anchorage independent proliferation.
452. The engineered cell of any one of embodiments 408-448, wherein absence of the stimulus causes anchorage independent proliferation.
453. The engineered cell of any one of embodiments 409-444, wherein the stimulus is selected from the group consisting of a change in a presence, absence, or level of: an antibiotic, a protein, a chemical compound, a salt of any one of these and any combination thereof.
454. The engineered cell of any one of embodiments 409-453, wherein the at least partially anchorage independent proliferation is at least partially a result of an increase in expression of: integrin-linked kinase (ILK), cyclin D1, Cdk4, ST6 N-Acetylgalactosaminide Alpha-2, 6-Sialytransferase 5 (ST6GALNAC5), or a combination thereof.
455. The engineered cell of any one of embodiments 408-454, wherein the engineered cell is grown in a bioreactor.
456. The engineered cell of any one of embodiments 409-455, wherein the at least partially anchorage independent proliferation is at least partially in a suspension and wherein the anchorage dependent proliferation is at least partially on, in, or around a scaffold.
457. The engineered cell of embodiment 456, wherein the scaffold is at least partially natural or synthetic.
458. The engineered cell of embodiment 456 or 457, wherein the scaffold comprises a silk, a polylactide, a polyglycolide, a polyester, a polycaprolactone, a chitosan, a hydrogel or a combination thereof.
459. An isolated tissue comprising the engineered cell of any one of embodiments 408-458.
460. The isolated tissue of embodiment 459, wherein the tissue comprises a plurality of polyester fibers.
461. The isolated tissue of embodiment 459, wherein at least a portion of the tissue is tanned.
462. The isolated tissue of 462, wherein the at least a portion of the tissue is tanned with a tanning agent comprising: a chromium, an aluminum, a zirconium, a titanium, an iron, a sodium aluminum silicate, a formaldehyde, a glutaraldehyde, an oxazolidine, an isocyanate, a carbodiimide, a polycarbamoyl sulfate, tetrakis hydroxyphosphonium sulfate, a sodium p-[(4,6-dichloro-1,3,5-triazin-2-yl)amino] a benzenesulphonate, a pyrogallol, a catechol, a syntan, or any combination thereof.
463. The isolated tissue of embodiment 461, wherein at least a portion of the tissue further comprises an extracellular matrix.
464. A leather comprising at least a portion of the engineered cell, a derivative thereof, or a progeny thereof of any one of embodiments 408-458, or the isolated tissue of embodiment 459-463.
465. The leather of embodiment 464, wherein the leather is in the form of any one selected from the group consisting of a bag, a belt, a watch strap, a package, a shoe, a boot, a footwear, a glove, a clothing, a vest, a jacket, a pant, a hat, a shirt, an undergarment, a luggage, a clutch, a purse, a ball, a backpack, a wallet, a saddle, a harness, a chap, a whip, furniture, furniture accessories, an upholstery an automobile car seat, an automobile interior, and any combination thereof.
466. The leather of embodiment 464, wherein the leather comprises a biofabricated material.
467. The leather of embodiment 466, wherein the biofabricated material comprises zonal properties.
468. The engineered cell of any one of embodiments 408-458, wherein the engineered cell comprises any one selected from the group consisting of a c-MycER system, a Tet-on system, a Tet-off system and a combination thereof.
469. The engineered cell of any one of embodiments 408-458 or 468, wherein the engineered cell comprises any one selected from the group consisting of a Cre-LoxP system, TALENS, Zinc Finger, a CRISPR system or a component thereof, and any combination thereof.
470. The engineered cell of any one of embodiments 408-458 or 468-469, wherein the polynucleotide encoding the at least partially reversible exogenous molecular switch comprises DNA, RNA, or a combination thereof.
471. The engineered cell of embodiment 470, wherein the polynucleotide encoding the at least partially reversible exogenous molecular switch comprises the DNA.
472. The engineered cell of embodiment 471 or 470, wherein the polynucleotide encoding the at least partially reversible exogenous molecular switch comprises cDNA.
473. The engineered cell of embodiment 470, wherein the polynucleotide encoding the at least partially reversible exogenous molecular switch comprises the RNA.
474. The engineered cell of embodiment 473, wherein the polynucleotide encoding the at least partially reversible exogenous molecular switch comprises mRNA.
475. The engineered cell of any one of embodiments 408-458 or 468-474, wherein the polynucleotide encoding the at least partially reversible exogenous molecular switch comprises an inducible promotor or operator, wherein the promoter or operator is configured to repress or activate expression of a gene.
476. The engineered cell of embodiment 475, wherein the promotor or operator comprises any one selected from the group consisting of a tetracycline-controlled transcriptional unit, a dexamethasone-controlled transcriptional unit, a doxycycline-controlled transcriptional unit, a C-mycR transcriptional controlled unit and any combination thereof.
477. The engineered cell of any one of embodiments 408-458 or 468-476, wherein the polynucleotide encoding the at least partially reversible exogenous molecular switch is codon optimized.
478. The engineered cell of any one of embodiments 408-458 or 468-476, wherein the polynucleotide encoding the at least partially reversible exogenous molecular switch comprises an epigenetically modified base.
479. The engineered cell of embodiment 478, wherein the epigenetically modified base comprises a pyrimidine.
480. The engineered cell of embodiment 479, wherein the pyrimidine is a cytosine or a thymine.
481. The engineered cell of any one of embodiments 478-480, wherein the epigenetically modified base comprises any one selected from the group consisting of a methylated base, a hydroxymethylated base, a formylated base, and a carboxylic acid containing base.
482. The engineered cell of embodiment 481, wherein the epigenetically modified base comprises the hydroxymethylated base.
483. The engineered cell of any one of embodiments 481-482, wherein the hydroxymethylated base comprises a 5-hydroxymethylated base.
484. The engineered cell of embodiment 483, wherein the 5-hydroxymethylated base comprises a 5-hydroxymethylcytosine.
485. The engineered cell of embodiment 481, wherein the epigenetically modified base comprises the methylated base.
486. The engineered cell of embodiment 485, wherein the methylated base comprises a 5-methylated base.
487. The engineered cell of embodiment 486, wherein the 5-methylated base comprises a 5-methylcytosine.
488. A method comprising contacting the engineered cell of any one of embodiments 408-458 or 468-487 with a stimulus.
489. The method of embodiment 488, wherein the stimulus is an environmental stimulus.
490. The method of any one of embodiments 488-489, wherein the stimulus comprises any one selected from the group consisting of a change in: pH, light, temperature, an electric current, microenvironment, the presence or absence of ions, a change in mechanical stimulus, the level of an ion, the presence of one or more ion type, and any combination thereof.
491. The method of embodiment 490, wherein the stimulus comprises the change in the temperature.
492. The method of embodiment 491, wherein the engineered cells are grown at a temperature for the at least partially anchorage independent proliferation at from about 28° C. to about 34° C.
493. The method of embodiment 492, wherein removal of the stimulus abates the at least partially anchorage independent proliferation.
494. The method of embodiment 491, wherein the engineered cells are grown at a temperature for the at least partially anchorage dependent proliferation at from about 37° C. to about 41° C.
495. The method of embodiment 494, wherein removal of the stimulus abates the at least partially anchorage dependent proliferation.
496. The method of any of embodiments 489-495, wherein the at least partially reversible exogenous molecular switch or the polynucleotide encoding the at least partially reversible exogenous molecular switch is introduced into the engineered cell by transfection, electroporation, or transduction.
497. The method of any of embodiments 489-496, wherein the at least partially reversible exogenous molecular switch or the polynucleotide encoding the at least partially reversible exogenous molecular switch is introduced by any one selected from the group consisting of a vector, wherein the vector is a virus, a viral-like particle, an adeno-associated viral vector, a liposome, a nanoparticle, a plasmid, linear dsDNA and a combination thereof.
498. The method of any one of embodiments 497, wherein the vector comprises the plasmid.
499. The method of any one of embodiments 488-498, wherein proliferation of the engineered cell is determined at least in part by a method selected from the group consisting of manual cell counting, automated cell counting and indirect cell counting.
500. The method of embodiment 399, wherein proliferation of the engineered cell is determined by use of a method selected from the group consisting of a counting chamber, colony forming unit counting, electrical resistance, flow cytometry, image analysis, stereologic cell counting, spectrophotometry, and impedance microbiology.
501. A method comprising tanning at least a portion of a tissue, wherein the tissue comprises the engineered cell of any one of embodiments 408-458 or 468-487.
502. The method of embodiment 401, wherein the tissue comprises a layered structure.
503. The method of embodiment 402, wherein the layered structure comprises any one selected from the group consisting of a dermal layer, an epidermal layer, laminin, fibronectin, collagen and a combination thereof.
504. The method of any one of embodiments 401-403, wherein the tissue comprises any one selected from the group consisting of a fibroblast, a keratinocyte, a corneocyte, a melanocyte, a Langerhans cell, a basal cell, an adipocyte, a smooth muscle cell, an epithelial cell and a combination thereof.
505. A method of selecting or screening for the engineered cell of any one of embodiments 408-458 or 468-487.
506. A synthetic leather, which prior to tanning comprises a portion of a tissue, wherein the tissue comprises the engineered cell of any one of embodiments 408-458 or 468-487.
507. The synthetic leather of embodiment 506, wherein the tissue is at least partially subjected to further processing.
508. The synthetic leather of embodiment 507, wherein the further processing is selected from the group consisting of tanning, preserving, soaking, bating, pickling, depickling, thinning, retanning, lubricating, crusting, wetting, sammying, shaving, rechroming, neutralizing, dyeing, fatliquoring, filling, stripping, stuffing, whitening, fixating, setting, drying, conditioning, milling, staking, buffing, finishing, oiling, brushing, padding, impregnating, spraying, roller coating, curtain coating, polishing, plating, embossing, ironing, glazing, tumbling, and any combination thereof.
509. The synthetic leather of embodiment 506, wherein the synthetic leather comprises a biofabricated material, wherein the biofabricated material comprises the engineered cell.
510. The synthetic leather of embodiment 509, wherein the biofabricated material comprises zonal properties.
511. A culture vessel comprising the engineered cell of any one of embodiments 109-159, 169-188, 279-286-286, 408-458, or 468-487.
512. The culture vessel of embodiment 511, wherein the culture vessel comprises any one selected from the group consisting of a plastic, a metal, a glass and a combination thereof.
513. The culture vessel of embodiment 511, wherein the culture vessel comprises an agent that causes the engineered cell to adhere to at least a portion of the culture vessel.
514. The culture vessel of embodiment 513, wherein the agent comprises poly-L-lysine.
515. A manufacturing facility comprising the engineered cell of any one of embodiments 408-458 or 468-487.
516. A kit comprising the engineered cell of any one of embodiments 408-458 or 468-487.
517. The kit of embodiment 516, further comprising a growth medium.
518. The kit of any one of embodiments 516 or 517, wherein the kit further comprises instructions for use.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-75. (canceled)

76. A method of forming tanned synthetic leather comprising:
a) introducing an exogenous polynucleotide into a fibroblast or fibroblast-like cell;
b) seeding the fibroblast or the fibroblast-like cell on a scaffold to form a cell layer; and
c) tanning a collagenous extracellular matrix comprising collagen secreted by the fibroblast or the fibroblast-like cell to generate the tanned synthetic leather.

77. The method of claim 76, wherein the fibroblast or the fibroblast-like cell comprises a bovine fibroblast.

78. The method of claim 76, wherein the introducing increases:
a) collagen production,
b) proliferation, or
c) any combination of (a) and (b);
wherein (a), (b), or (c) is relative to a control fibroblast or control fibroblast-like cell that is otherwise identical to the fibroblast or the fibroblast-like cell but lacks the exogenous polynucleotide.

79. The method of claim 78, wherein the collagen production is determined by microscopy, Sircol red assay, or a combination thereof.

80. The method of claim 78, wherein the proliferation is determined by manual cell counting, automated cell counting, indirect cell counting, or a combination thereof.

81. The method of claim 76, wherein the exogenous polynucleotide encodes a polypeptide or a biologically active fragment thereof, that alters telomerase activity or activity of a tumor suppressor protein.

82. The method of claim 76, wherein the exogenous polynucleotide encodes:
a) an SV40 large T antigen (SV40-TAg) polypeptide or a biologically active fragment thereof; or
b) a telomerase (TERT) protein or a biologically active fragment thereof.

83. The method of claim 82, wherein the exogenous polynucleotide encodes the telomerase (TERT) protein or the biologically active fragment thereof.

84. The method of claim 76, wherein the introducing immortalizes the fibroblast or the fibroblast-like cell.

85. The method of claim 76, wherein the scaffold comprises silk fibroin, cellulose, cotton, acetate, acrylic, latex fiber, linen, nylon, rayon, velvet, modacrylic, olefin polyester, polylactic acid (PLA), saran, vinyon, wool, jute, hemp, bamboo, flax, or a combination thereof.

86. The method of claim 85, wherein the scaffold comprises a rayon.

87. The method of claim 76, wherein the collagenous extracellular matrix comprises collagen type I, collagen type III, elastin, fibronectin, laminin, or a combination thereof.

88. The method of claim 76, wherein the cell layer is decellularized.

89. The method of claim 76, wherein the tanning comprises vegetable tanning, chrome tanning, aldehyde tanning, syntan tanning, bacterial dyeing, or a combination thereof.

90. The method of claim 76, further comprising introducing a mutation into the fibroblast or the fibroblast-like cell.

91. The method of claim 90, wherein the mutation is in a cell cycle gene, an oncogene, a metabolic gene, or a combination thereof.

92. The method of claim 76, further comprising contacting the fibroblast or the fibroblast-like cell with medium comprising:
a) L-ascorbic acid 2-phosphate or a pharmaceutically acceptable salt thereof;
b) Transforming Growth Factor beta 1 or a biologically active fragment thereof; or
c) a combination thereof.

93. An artificial dermal layer comprising:
a) a bovine fibroblast or bovine fibroblast-like cell, wherein the bovine fibroblast or the bovine fibroblast-like cell comprises an exogenous polynucleotide;
b) a scaffold, wherein the bovine fibroblast or the bovine fibroblast-like cell is at least partially in contact with the scaffold, and wherein the scaffold comprises a rayon;
c) an extracellular matrix, wherein the extracellular matrix comprises collagen type I, collagen type III, or a combination thereof; and
d) an at least partially decellularized cell layer.

94. The artificial dermal layer of claim 93, wherein the exogenous polynucleotide encodes:
a) an SV40 large T antigen (SV40-TAg) polypeptide or a biologically active fragment thereof; or
b) a telomerase (TERT) protein or a biologically active fragment thereof.

95. The artificial dermal layer of claim 94, wherein the exogenous polynucleotide encodes the telomerase (TERT) protein or the biologically active fragment thereof.
